(19) United States
(12) Patent Application Publication
(10) Pub. No.: US 2023/0272097 A1
(43) Pub. Date: Aug. 31, 2023

(54) THERAPEUTIC ANTIBODIES

(71) Applicant: PETMEDIX LTD, Babraham, Cambridge (GB)

(72) Inventors: Allan BRADLEY, Cambridge (GB); Andrew James THOMPSON, Cambridge (GB); Daniel John BOLLAND, Cambridge (GB); Juexuan WANG, Cambridge (GB); Roberto BANDIERA, Cambridge (GB); Steve HOLMES, Cambridge (GB)

(21) Appl. No.: 18/007,314

(22) PCT Filed: Aug. 6, 2021
(86) PCT No.: PCT/GB2021/052043

§ 371 Date: Jan. 30, 2023

(30) Foreign Application Priority Data
Aug. 7, 2020 (GB) 2012331.1

(51) Int. Cl. C07K 16/28; A61K 45/06; C07K 14/705

(52) U.S. Cl. CPC C07K 16/2875; A61K 45/06; C07K 14/70578; C07K 2317/76; C07K 2317/734; C07K 2317/24; C07K 2317/92

(57) ABSTRACT

The invention relates to antibodies which modulate the OX40 signalling pathway to treat inflammatory diseases in companion animals, for example atopic dermatitis (AD) and/or eczema. Related methods and uses are also described.

### INTRODUCTION

The present invention relates to antibodies which modulate the OX40 signalling pathway to treat inflammatory diseases in companion animals.

Atopic dermatitis (AD) and/or eczema, characterised by chronic, dry, itchy, red skin, is a significant problem in dogs, effecting 10-15% of pet dogs.

Cytopoint is an existing treatment for Atopic Dermatitis in dogs that has a recommended minimum dose of 1 mg kg⁻¹, by injection, once a month. Cytopoint is an anti-IL31 Ab (described for example in WO2013/011407A1 and WO2019/177697), specifically intended to treat the itch (pruritus) associated with atopic dermatitis. However, there are at least one third of canine AD patients that do not satisfactorily respond to Cytopoint, and in some instances, efficacy can decrease following the first injection (see, for example “CVMP assessment report for CYTOPOINT EMA/118401/2017 (EMEA/V/C/003939/0000)”, and World Association for Veterinary Dermatology, Cytopoint roundtable May 2017, https://wavd.org/wp-content/uploads/cytopoint-roundtable-2017-05.pdf). The most common side effects of Cytopoint (which may affect up to 1 in 1,000 animals) are allergic reactions such as anaphylaxis, facial oedema and urticaria. Cytopoint must not be given to dogs weighing less than 3 kg. (https://www.ema.europa.eu/en/documents/product-information/cytopoint-epar-product-information_en.pdf).

There is a need for improved treatments as well as for drugs that treat the underlying cause of the disease rather than the symptoms. In particular, there is a need for a treatment that is safe, has a long duration of action, and has efficacy to cover a wider spectrum of patients, particularly non-responders.

Advantageously, targeting OX40/OX40L which are upstream in the inflammatory cascade, offers the possibility to modulate multiple cytokines simultaneously.

Canine OX40L is described in U.S. Pat. No. 10,196,435. The protein sequence of canine OX40 has not been reported in the scientific or patent literature to date.

A monoclonal antibody (7D6) that binds feline CD134 (OX40) and its effect on the felineimmunodeficiency virus is described in Willett et al, Journal of Virology, 81 (18), 2007, pages 9665-9679.

The use of an antibody to OX40L or OX40 in the treatment of immune-regulated diseases, such as atopic dermatitis, in companion animals (e.g. dogs) has not been shown before.

### STATEMENTS OF INVENTION

In a first aspect, the invention relates to an antibody or fragment thereof that specifically binds to companion animal OX40L or to companion animal OX40.

The companion animal may be a dog or a cat.

In one embodiment, the antibody or fragment binds to canine OX40L.

In one embodiment, the antibody or fragment is capable of
a) Reducing, inhibiting or neutralising OX40 activity or activation in the companion animal or in a cell of the companion animal;
b) Modifying secretion of a cytokine in the companion animal or in a cell of the companion animal and/or
c) Decreasing proliferation of leukocytes in the companion animal or in a cell of the companion animal.

In one embodiment, the antibody or fragment is capable of
a) Reducing, inhibiting or neutralising OX40 activity or activation in the companion animal or in a cell of the companion animal;
b) Decreasing secretion of inflammatory cytokine in the companion animal or in a cell of the companion animal and/or
c) Decreasing secretion of an inflammatory chemokine or chemokine receptor in the companion animal or in a cell of the companion animal and/or
d) Increasing the secretion of suppressive cytokine(s) in the companion animal or in a cell of the companion animal and/or
e) Increasing the secretion of suppressive chemokines(s) or chemokine receptors in the companion animal or in a cell of the companion animal and/or
f) Decreasing proliferation of leukocytes in the companion animal.

Suitable assays assessing these properties, such as a Mixed Lymphocyte Reaction (MLR) assay or HEK-blue assay for measuring an inhibition of NFkB activity, are described herein, such as the assays shown in the examples, e.g. the PBMC activation assay. Other assays are known to the skilled person and may also be used.

The cytokine or cytokine receptor may be selected from TNF alpha, IL-1Ra, IL-2, IL-3, IL-4, IL-5, IL-6, IL-8, IL-9, IL-10, IL-13, IL-17, RANTES, GM-CSF, TGF-β and interferon gamma.

In one embodiment, the antibody or fragment binds to canine OX40. The antibody or fragment may be capable of reducing, inhibiting or neutralising OX40 activity or activation in the companion animal or in a cell of the companion animal.

In one embodiment of the forgoing and the various aspects of the invention relating to antibodies that bind OX40 or OX40L, the antibody or fragment is a fully canine, chimeric or caninized antibody. The terms fully canine and canine are used interchangeably herein. According to a preferred embodiment, the antibody is canine (i.e. fully canine).

For example, said fragment is selected from a F(ab′)2, Fab, Fv, scFv, heavy chain, light chain, variable heavy (VH), variable light (VL) chain, CDR region, single VH or VL domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

In one embodiment, the antibody or fragment is conjugated to another moiety. The antibody or fragment may comprise a therapeutic moiety, half life extending moiety or label.

In another aspect, the invention relates to a binding molecule comprising an antibody or fragment as described above.

In another aspect, the invention relates to an antibody or fragment or the binding molecule as described above for use in the treatment of a disease.

In another aspect, the invention relates to a pharmaceutical composition comprising an antibody or fragment thereof or binding molecule as described above.

In another aspect, the invention relates to an antibody or fragment thereof, binding molecule pharmaceutical as described above for use in the treatment of an OX40 or OX40L-mediated disease.

In another aspect, the invention relates to methods of treating or preventing an OX40 or OX40L-mediated disease comprising administering to a subject in need thereof an antibody or fragment, binding molecule or the pharmaceutical composition as described above.

For example, the disease is selected from an inflammatory or autoimmune disease.

The disease may be an inflammatory skin diseases, including atopic dermatitis, allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma, bacterial pneumonia, antigen-antibody complex mediated diseases; inflammations of the lung, including pleurisy, alveolitis, vasculitis, pneumonia, chronic bronchitis, bronchiectasis, and cystic fibrosis.

In one embodiment, the antibody or fragment, binding molecule, pharmaceutical composition is administered together with one or more therapeutic agent.

For example, said one or more therapeutic agent is selected from rapamycin (sirolimus), tacrolimus, cyclosporine (e.g. Atopica®), corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa (TNFα)/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, PDE4 inhibitors, lokietmab (Cytopoint®), and oclacitinib (Apoquel®).

In another aspect, the invention relates to a method of decreasing the secretion of cytokines comprising administering to a subject in need thereof an antibody or fragment, binding molecule or a pharmaceutical composition as described above.

In another aspect, the invention relates to a multispecific binding agent comprising an antibody or fragment or a binding molecule as described above.

In another aspect, the invention relates to a combination therapy comprising antibody or fragment, binding molecule or pharmaceutical composition as described above.

In another aspect, the invention relates to an immunoconjugate comprising an antibody or fragment or binding molecule as described above.

In another aspect, the invention relates to a kit comprising an antibody or fragment thereof, binding or pharmaceutical composition as described above.

In another aspect, the invention relates to an isolated canine OX40 protein comprising SEQ ID NO. 4 or 6 or a variant thereof.

In another aspect, the invention relates to isolated nucleic acid molecule encoding a protein as above, optionally comprising SEQ ID NO. 3 or 5 or a variant thereof.

In another aspect, the invention relates to vector comprising a nucleic acid as above.

In another aspect, the invention relates to host cell comprising a nucleic acid or a vector as described above where the host cell is optionally selected from a mammalian, yeast, plant or bacterial cell.

In another aspect, the invention relates to a method for detecting OX40L or OX40 in a companion animal comprising contacting a test sample with an antibody or fragment or a binding molecule as described above.

In another aspect, the invention relates to a trimeric soluble companion animal OX40L extra cellular domain probe and its use in in a method of screening for companion animal OX40L antibodies.

The invention is described in the following non-limiting figures and tables.

### FIGURES

FIG. 1. OX40 and OX40L nucleotide sequences. A. The predicted number and arrangement of exons within the dog genome. Exons are represented by boxes. Numbers above the line represent the predicted size of each exon in nucleotides. Numbers below the line represent the predicted size of introns in nucleotides, 5′ UTR and 3′ UTR (shaded boxes). Predictions were made using NCBI Assembly ID: 317138 (CanFam3.1). The size and sequence of the predicted coding sequences was confirmed by RT-PCR. B. Cartoon representations of some of the constructs used in this study.

FIG. 2. Nucleotide sequences of the two OX40 splice variants. A. The nucleotide sequence of the full-length splice variant consisting of exons 1-7 (SEQ ID NO. 157). B. A second splice variant lacks exon 6 (uppercase text) (SEQ ID NO. 158). C. An alignment of the corresponding amino acids (SEQ ID Nos. 4 and 6) is shown below. Based on homology with OX40 protein from other species, exon 6 is predicted to contain the transmembrane domain. In both panels the predicted 5′UTR's and 3′UTR's are shown in lowercase italics. Cartoon representations of these sequences are shown in FIG. 1.

FIG. 3. Relative abundance of the two splice variants in PHA-activated dog PBMC's, as determined by a diagnostic PCR of non-biased sub-cloned RT-PCR products. Measurements were taken on independent PBMC samples, 1 and 4 days after activation. Insert: PCR discrimination of short (S) and long (L) splice variants. +ve=positive control. 1 kb Plus DNA Ladder (NEB), with brighter bands at 0.5, 1.0 and 3.0 bp.

FIG. 4. Serum antibody titres from immunised and non-immunised mice, measured using flow cytometry. A. In non-immunised mice there was no appreciable antibody binding to untransfected cells (Δ) or those stably expressing OX40L (◯). B. Serum from immunised mice was collected 10-days after the first boost and yielded a clear distinction between untransfected cells (∇) and those stably expressing OX40L (□). In this example, hydrodynamic tail vein injection was used for the prime immunisation and OX40L was stably expressed in mouse embryonic fibroblasts for subsequent cell-based boosts.

FIG. 5. Functional assay to measure OX40-OX40L interactions. A. Representative curves showing the enzymatic activity of secreted alkaline phosphatase, that was liberated 6 hrs, 24 hrs and 48 hrs after mixing HEK-Blue-OX40 with HEK-OX40L cells. All curves are baseline subtracted with the signal obtained with culture media alone.

FIG. 6. Nucleotide sequence of the OX40L. A. The full-length sequence cloned from dog, with predicted UTR's shown in italics (SEQ ID NO. 159). B. Secreted OX40L construct containing an IL-2 signal sequence (capital letters, underlined), His-tag (capital letters, shaded), AviTag (capital letters, bold), leucine zipper (lowercase letters, shaded), a GS linker (capital letters, bold, underlined) and the extracellular domain of OX40L, truncated at the extracellular-transmembrane domain junction (SEQ ID NO. 160). Cartoon representations of these sequences are shown in FIG. 1.

FIG. 7. Schematic representation of soluble proteins containing OX40L extra-cellular domain (ECD). A. Monomeric OX40L probe containing monomeric human IgG1 (mvhfc), 6×histidine (HIS) tag and tobacco etch virus (TEV) protease cleaving peptide. B. Trimeric OX40L probes containing chicken tenascin C trimerization domain and either human IgG1 Fc or HIS tags. The term trimeric refers to the conformation of OX40L extra cellular domain.

FIG. 8. Single dose cell binding assay of candidate OX40L antibodies. Histograms show the overlayed intensity of signal obtained by flow cytometry of OX40L expressing HEK293 cells or the parental line stained with candidate OX40L antibodies and subsequently with a fluorescently labelled secondary antibody. All antibodies shown in the figure bind OX40L-expressing HEK cells with higher affinity compared to the parental line.

FIG. 9. Density plot showing flow cytometry of OneComp eBeads loaded with PMX014 and PMX020 (or no antibody as control, left panel) and subsequently stained with OX40L monomeric (top panels) or trimeric (Fc fusion) probes (bottom panels). While PMX014 binds both probes, PMX020 only binds the trimeric probe.

FIG. 10. Affinity determination for antibodies that bind OX40L on HEK cells expressing OX40L. Graphs are generated by plotting GeoMean arbitrary units (AU) against Log₁₀ [concentration]. K_{d} (EC₅₀) was calculated using Prism. K_{d} (PMX 012)=3.0017e-9 M; K_{d} (PMX 013)=1.653e-7 M; K_{d} (PMX 014)=5.122e-9 M; K_{d} (PMX 016)=3.743e-8 M; K_{d} (PMX 017)=3.004e-8 M; K_{d} (PMX 018)=2.360e-8 M; K_{d} (PMX 019)=4.488e-9 M; K_{d} (PMX 020)=2.803e-9 M.

FIG. 11. Affinity determination for antibodies that bind OX40L on MEF cells expressing OX40L. Graphs are generated by plotting GeoMean arbitrary units (AU) against Log₁₀ [concentration]. K_{d} (EC₅₀) was calculated using Prism. K_{d} (PMX 013)=2.272e-8 M; K_{d} (PMX 014)=1.111e-9 M; K_{d} (PMX 016)=1.078e-7 M; K_{d} (PMX 017)=1.068e-8 M; K_{d} (PMX 019)=3.826e-8 M; K_{d} (PMX 020)=7.101e-10 M.

FIG. 12. Affinity determination for antibodies that bind OX40L formulated in canine IgGB Def2 and Def7 backbones. The experiment was performed using HEK cells expressing OX40L. Graphs are generated by plotting GeoMean arbitrary units (AU) values against Log₁₀ [concentration]. K_{d} (EC₅₀) was calculated using Prism. K_{d} (PMX014 Def2)=7.462e-008 M; K_{d} (PMX014 Def7)=7.424e-008 M; K_{d}(PMX020 Def2)=4.539e-008 M; K_{d} (PMX020 Def7)=5.813e-008 M. Def2 or Def7 backbones do not change the affinity of the variable region.

FIG. 13. Single-concentration blocking assay (HEK blue assay) for antibodies that bind OX40L. Data are normalised with a maximum signal (100%) defined by the response obtained in the absence of antibodies and a minimum signal (0%) measured in the presence of HEK-blue-OX40 cells alone. Statistical test, Dunnett's multiple comparison test, adj p<0.0001 (****), adj p=0.0063(**).

FIG. 14. Concentration-dependent block (HEK blue assay) by antibodies that bind OX40L. Data are normalised with a maximum signal (100%) defined by the response obtained in the absence of antibodies and a minimum signal (0%) measured in the presence of HEK-blue-OX40 cells alone.

FIG. 15. Alignment of heavy chain variable region protein sequences of PMX012 to PMX023. Residues in grey are conserved among all aligned sequences.

FIG. 16. Alignment of light chain variable region protein sequences of PMX012 to PMX023. Residues in grey are conserved among all aligned sequences.

FIG. 17. Alignment of heavy chain variable region protein sequences for antibodies that bind OX40L, categorised by homology. Residues in grey are conserved among all aligned sequences in each group.

FIG. 18. Alignment of light chain variable region protein sequences for antibodies that bind OX40L, categorised by homology. Residues in grey are conserved among all aligned sequences in each group.

FIG. 19. shows CDC activity of IgG B mutants: Def mutants repress IgG-B CDC activity. Complement-dependent cytotoxicity assay showing the reduced complement dependent killing of canine T cells expressing human CD20 (CLBL1 hCD20) by effector function deficient IgG-B mutants Def 1, 2, 3, 5, 6, 7, 8 and 9, when compared to wild type (WT) canine IgG-B. All antibodies used in this assay have Ofatumumab variable regions. Data are plotted as percentage of killing where 100% means all cells are killed and 0% means signal was identical to what obtained in control cells (no antibody added).

Table 1. Amino Acid Residues and Examples of Conservative Amino Acid Substitutions.

Table 2. Nucleic acid and amino acid sequences.

Table 3. Affinity (K_{d}) of PMX012, PMX014 and PMX023 as determined in the indicated experiment. SPR data only indicates K_{d} values at equilibrium.

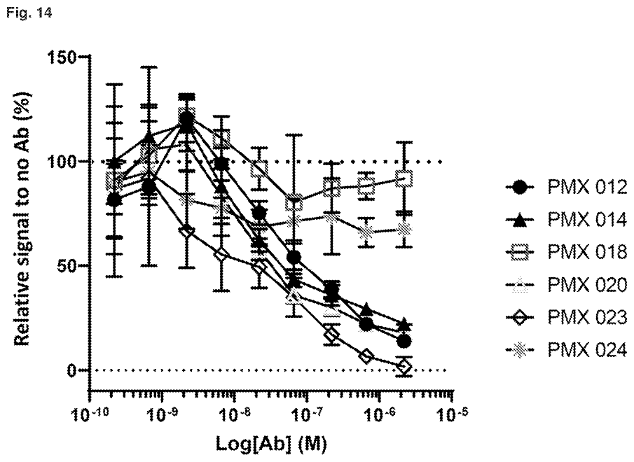

Table 4. IC50 values derived from inhibition of OX40 signalling activation in concentration dependent HEK blue assay as described in FIG. 14.

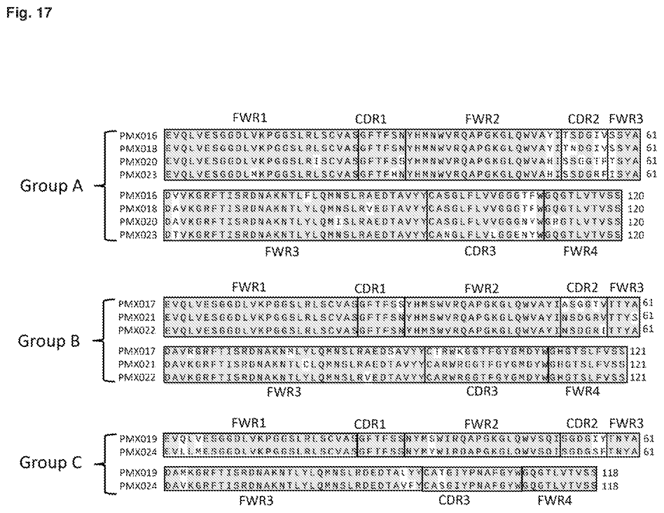

Table 5: Percent of conserved amino acids in heavy chain variable regions of PMX012 to PM024 antibodies (all) or within each group as described in FIG. 17. Higher homology is observed within each group.

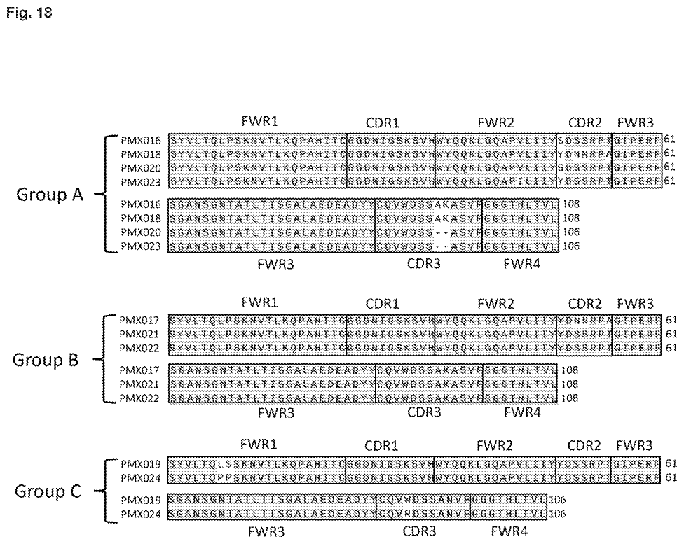

Table 6: Percent of conserved amino acids in light chain variable regions of PMX012 to PM024 antibodies (all) or within each group as described in FIG. 18. Higher homology is observed within each group.

### DETAILED DESCRIPTION

OX40 is expressed on the surface of T-cells, and OX40L on both the surface of T-cells and antigen presenting cells such as B cells and macrophages. Neither OX40 nor OX40L are constitutively expressed, but increase 24-72 hours following activation of their respective cells. OX40L binding to OX40 receptors on T-cells increases T-cell cytokine production and prevents T-cells from subsequently dying. OX40 therefore has a critical role in establishing and maintaining an immune response.

In humans, both the density of OX40L and the number of OX40 positive cells is significantly greater in the lesional dermis than in the healthy-looking dermis in atopic dermatitis, and blockade of OX40/OX40L signalling modulates several pro-inflammatory responses. OX40L also controls the response of dendritic cells to thymic stromal lymphopoietin (TSLP), which results in IL-21 and CXCL13 production. The OX40/OX40L axis consequently offers the possibility of modulating multiple pro-inflammatory responses, as its co-stimulatory signal sits upstream of several cellular processes that release pro-inflammatory cytokines.

OX40 signalling has been linked to various diseases such as allergy, asthma, and diseases associated with autoimmunity and inflammation, which includes multiple sclerosis, rheumatoid arthritis, inflammatory bowel disease, graft-versus-host disease, experimental autoimmune encephalomyelitis (EAE), experimental leishmaniasis, collagen-induced arthritis, colitis (such as ulcerative colitis), contact hypersensitivity reactions, diabetes, Crohn's Disease, and Grave's Disease. Evidence in humans suggests that disruption of the OX40/OX40L axis reduces proliferative responses and could be used to treat or ameliorate the symptoms of a number of diseases, including atopic dermatitis and cancer. Antibodies to either OX40 or OX40L could be used to achieve this disruption

However, the role of antibody treatments for OX40/OX40L mediated diseases in companion animals, such as atopic dermatitis, e.g. in dogs, has not been investigated previously.

Thus, in a first aspect, the invention relates to an antibody or fragment thereof that specifically binds to companion animal OX40L or to companion animal OX40.

Companion animals of the invention are suitably selected from dogs, cats, horses, birds, rabbits, goats, reptiles, fish and amphibians. A dog is a preferred companion animal of the invention. A cat is a preferred companion animal of the invention. A horse is a preferred companion animal of the invention. For the avoidance of doubt, a human is not a companion animal.

In one aspect, the companion animal is a dog.

In one aspect, the companion animal is a cat.

In one aspect, the companion animal is a horse.

In one embodiment, antibodies and fragments described herein bind specifically to wild type canine OX40L. The amino acid sequence (SEQ ID No.1) and nucleotide sequences for wild type canine OX40L are shown in Table 2 (SEQ ID No. 2). Antibodies and fragments described herein bind specifically to SEQ ID No.1. In one embodiment, antibodies and fragments described herein bind specifically to variants of SEQ ID No.1.

In one embodiment, antibodies and fragments described herein bind specifically to wild type canine OX40. The amino acid sequence (SEQ ID Nos. 4 and 6) and nucleotide sequences for wild type canine OX40 are shown in table 2 (SEQ ID Nos. 3 and 5). As explained in the examples, two different splice variants were identified. Antibodies and fragments described herein bind specifically to proteins codified by SEQ ID No. 3 and/or 5. In one embodiment, antibodies and fragments described herein bind specifically to proteins codified by variants of SEQ ID No. 3 and/or 5 (SEQ ID Nos. 4 and 6).

Variants of the sequences described above may have at least 75%, 80%, 85%, 90% or 95% sequence identity to the sequences shown above.

As used herein, the terms “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and in some embodiments not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percentage identity between two amino acid sequences can be determined using well known mathematical algorithms.

Unless otherwise specified, the term OX40L as used herein refers to a companion animal OX40L, e.g. dog or cat OX40L. OX40L is also known as “OX40 Antigen Ligand”, “OX40 Ligand”, “CD252”, “TNFSF4” and “CD134 Ligand”. In one embodiment, the antibody or fragment thereof binds to dog OX40L. In one embodiment, the antibody or fragment thereof binds to cat OX40L.

Unless otherwise specified, the term OX40 as used herein refers to a companion animal OX40, e.g. dog or cat OX40. OX40 is also known as “TNFR Superfamily Member 4”, “TNFRSF4”, “OX40 Antigen” and “CD134”. In one embodiment, the antibody or fragment thereof binds to dog OX40L. In one embodiment, the antibody or fragment thereof binds does not bind to cat OX40L. In one embodiment, the antibody or fragment thereof is not 7D6 as disclosed in Willett et al.

The terms “OX40L binding molecule/protein/polypeptide/agent/moiety”, “OX40L antigen binding molecule molecule/protein/polypeptide/agent/moiety”, “anti-OX40L antibody”, “anti-OX40L antibody fragment” all refer to a molecule capable of specifically binding to the companion animal OX40L, e.g. dog or cat OX40L antigen. The binding reaction may be shown by standard methods, for example with reference to a negative control test using an antibody of unrelated specificity.

The terms “OX40 binding molecule/protein/polypeptide/agent/moiety”, “OX40 antigen binding molecule molecule/protein/polypeptide/agent/moiety”, “anti-OX40 antibody”, “anti-OX40 antibody fragment” all refer to a molecule capable of specifically binding to the companion animal OX40, e.g. dog or cat OX40 antigen. The binding reaction may be shown by standard methods, for example with reference to a negative control test using an antibody of unrelated specificity.

In some embodiments, the antibody or fragment provided herein binds to an OX40L epitope that is a three-dimensional surface feature of a OX40L polypeptide. In some embodiments, the antibody or fragment provided herein binds to an epitope comprising a polypeptide from a single subunit, or a conformational epitope arising from a multimeric form, (e.g., an epitope in a monomeric or trimeric form of an OX40L polypeptide). A region of an OX40L polypeptide contributing to an epitope may be contiguous amino acids of the polypeptide or the epitope may come together from two or more non-contiguous regions of the polypeptide. A OX40L epitope may be present in (a) the trimeric form (“a trimeric OX40L epitope”) of OX40L, (b) the monomeric form (“a monomeric OX40L epitope”) of OX40L, (c) both the trimeric and monomeric form of OX40L, (d) the trimeric form, but not the monomeric form of OX40L, or (e) the monomeric form, but not the trimeric form of OX40L

For example, in some embodiments, the epitope is only present or available for binding in the trimeric form, but is not present or available for binding in the monomeric form by an anti-OX40L antibody. In other embodiments, the OX40L epitope is linear feature of the OX40L polypeptide (e.g., in a trimeric form or monomeric form of the OX40L polypeptide). Antibodies provided herein may specifically bind to (a) an epitope of the monomeric form of OX40L, (b) an epitope of the trimeric form of OX40L, (c) an epitope of the monomeric but not the trimeric form of OX40L, (d) an epitope of the trimeric but not the monomeric form of OX40L, or (e) both the monomeric form and the trimeric form of OX40L. In some embodiments, the antibodies provided herein specifically bind to an epitope of the trimeric form of OX40L but do not specifically bind to an epitope the monomeric form of OX40L. In some embodiments, the antibodies provided herein bind to an epitope of the monomeric form of OX40L and may or may not bind to the trimeric form.

An antibody or fragment thereof “which binds” or is “capable of binding” an antigen of interest, e.g. companion animal OX40L or companion animal OX40 respectively, is one that binds the antigen with sufficient affinity such that the antibody or fragment is useful as a therapeutic agent in targeting a cell or tissue expressing the antigen OX40 or OX40L respectively as described herein.

Antibodies and fragments thereof as described herein bind specifically to the target companion animal OX40L or the target companion animal OX40 respectively. For example, in one embodiment, antibodies and fragments thereof as described herein bind specifically to canine OX40L. In another embodiment, antibodies and fragments thereof as described herein bind specifically to feline OX40L. For example, in one embodiment, antibodies and fragments thereof as described herein bind specifically to canine OX40. In another embodiment, antibodies and fragments thereof as described herein bind specifically to feline OX40. The term “specifically” in the context of antibody binding, refers to high avidity and/or high affinity binding of an antibody to a specific antigen, i.e., a polypeptide, or epitope. In many embodiments, the specific antigen is an antigen (or a fragment or subfraction of an antigen) used to immunize the animal host from which the antibody-producing cells were isolated.

In other words, binding to the OX40L or OX40 antigen is stronger than binding of the same antibody to other antigens, i.e. measurably different from a non-specific interaction. Thus, in one embodiment, the antibodies or fragments of the invention do not cross react with mouse or human OX40L or OX40 antigen.

The term “specific binding” or “specifically binds to” or is “specific for” a particular polypeptide or an epitope on a particular polypeptide target as used herein can be exhibited, for example, by a molecule having a KD (K_{d}) for the target of at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or lower. In one embodiment, the KD (K_{d}) is 10⁻⁹ M or lower. In one embodiment, the term “specific binding” refers to binding where a molecule binds to a particular polypeptide or epitope on a particular polypeptide without substantially binding to any other polypeptide or polypeptide epitope.

In one embodiment, antibodies of the invention are antagonistic antibodies that bind specifically to companion animal OX40L, for example canine OX40L.

As used herein, an “antagonist” or “inhibitor” of OX40L/OX40 refers to a ligand (e.g., antibody or fragment) that is capable of inhibiting or otherwise decreasing one or more of the biological activities of OX40L/OX40, such as in a cell expressing OX40L/OX40 or in a cell expressing an OX40L/OX40 ligand. For example, in certain embodiments, antibodies of the invention are antagonist antibodies that inhibit or otherwise decrease secretion of a cytokine from a cell having a cell surface-expressed OX40L/OX40 when said antibody is contacted with said cell. In some embodiments, an antagonist of OX40L (e.g., an antagonistic antibody of the invention) may, for example, act by inhibiting or otherwise decreasing the activation and/or cell signalling pathways of the cell expressing OX40L/OX40, thereby inhibiting a OX40L/OX40-mediated biological activity of the cell the relative to the OX40L/OX40-mediated biological activity in the absence of antagonist. In certain embodiments, the antibodies provided herein are fully canine, antagonistic anti-OX40L/OX40 antibodies, preferably fully canine, monoclonal, antagonistic anti-OX40L/OX40 antibodies.

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, CL.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can be of any type, class or subclass (e.g., for dogs IgG, IgE, IgM, IgD, IgA and IgY; e.g. canine IgG subtype, for example IgG-A, IgG-B, IgG-C, and IgG-D). In canine, there are four IgG heavy chains referred to as A, B, C, and D. These heavy chains represent four different subclasses of dog IgG, which are referred to as IgGA, IgGB, IgGC and IgGD. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). Amino acid sequences for IgG-A, IgG-B, IgG-C and IgG-D as used by the inventors and according to the aspects and embodiments of the invention are shown in Table 2.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme. The IMGT numbering scheme is described in Lefranc et al., Dev. Comp. Immunol., 29, 185-203 (2005). These terms, which are recognized in the art, refer to a system of numbering amino acid residues which are more variable (i.e., hypervariable) than other amino acid residues in the heavy and light chain variable regions of an antibody, or an antigen binding portion.

The IMGT numbering scheme is used herein unless otherwise specified.

The antibody to OX40L or OX40 according to the invention may be a canine, humanized, feline, chimeric antibody, felinized or caninized antibody.

A “chimeric antibody” is a recombinant protein that contains the variable domains including the complementarity determining regions (CDRs) of an antibody derived from one species, while the constant domains of the antibody molecule are derived from those of another species, e.g. a canine antibody. An exemplary chimeric antibody is a chimeric human—canine antibody.

A “humanized antibody” is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

As used herein, the term “caninized antibody” refers to forms of recombinant antibodies that contain sequences from both canine and non-canine (e.g., murine) antibodies. In general, the caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target. The non-canine sequences, e.g., of the hypervariable loops, may further be compared to canine sequences and as many residues changed to be as similar to authentic canine sequences as possible.

A “speciated” antibody (e.g. humanized, caninized, chimeric, felinized) is one which has been engineered to render it similar to antibodies of the target species. In one embodiment, a “speciated” antibody is greater than about 80%, 85% or 90% similar to antibodies of the target species.

In one embodiment, the antibody or antibody fragment is canine. By canine is meant fully canine. The terms fully canine and canine are used interchangeably herein.

In contrast to speciated antibodies, fully canine antibodies of the present invention have canine variable regions and do not include full or partial CDRs or FRs from another species. Advantageously, fully canine antibodies as described herein have been obtained from transgenic mice comprising canine immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine antibodies produced in this way generate antibodies with optimal properties for developability, minimizing lengthy lead optimization prior to production at scale. Advantageously, such fully canine antibodies present the lowest possible risk of immunogenicity when introduced into a patient animal which, in turn, facilitates a repeated dosing regimen. Adverse in vivo immunogenicity can be assessed, for example, by assays to identify the production of anti-drug antibodies (ADA), or a loss of efficacy over time in vivo. Given that ex vivo mAb engineering runs the risk of introducing development liabilities, immunogenicity, and reduced affinity (as outlined above), fully canine antibodies of the present invention are, therefore, most likely to be efficacious therapies in a clinical context.

The term “monoclonal antibody” as used herein refers to an antibody derived from a single B or plasma cell. All antibodies molecules in a monoclonal antibody preparation are identical except for possible naturally occurring post-translation modifications (e.g., isomerizations, amidations, carbohydrate addition) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “epitope” or “antigenic determinant” refers to a site on the surface of an antigen (to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody. The epitope may or may not be a three-dimensional surface feature of the antigen. In certain embodiments, an OX40L epitope is a three-dimensional surface feature of a OX40L polypeptide (e.g., in a trimeric form of a OX40L polypeptide). In other embodiments, a OX40L epitope is linear feature of a OX40L polypeptide (e.g., in a trimeric form or monomeric form of the OX40L polypeptide). Antibodies provided herein may specifically bind to an epitope of the monomeric form of OX40L, an epitope of the trimeric form of OX40L, or both the monomeric form and the trimeric form of OX40L. In specific embodiments, the antibodies provided herein specifically bind to an epitope of the trimeric form of OX40L but do not specifically bind the monomeric form of OX40L. In some embodiments, an antibody may, for example, bind to a monomer/single subunit and block formation of an active trimeric form. Suitably the antibodies bind to the extracellular domain of OX40L.

The term “antigen binding site” refers to the part of the antibody or antibody fragment that comprises the area that specifically binds to an antigen. An antigen binding site may be provided by one or more antibody variable domains. An antigen binding site is typically comprised within the associated V_{H} and V_{L} of an antibody or antibody fragment.

The term antibody as used herein also includes antibody fragments. Specifically, the invention also extends to antibody fragments. An antibody fragment is a portion of an antibody, for example a F(ab′)2, Fab, Fv, scFv, heavy chain, light chain, variable heavy (V_{H}), variable light (V_{L}) chain, CDR region, single V_{H} or V_{L} domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide. Therefore, an antibody fragment comprises an antigen binding portion.

Antibody fragments are functional fragments of a full-length antibody, that is they retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the VH and VL antibody domains connected into a single polypeptide chain.

scFv fragments (˜25 kDa) consist of the two variable domains, V_{H} and V_{L}. Naturally, V_{H} and V_{L} domain are non-covalently associated via hydrophobic interaction and tend to dissociate. However, stable fragments can be engineered by linking the domains with a hydrophilic flexible linker to create a single chain Fv (scFv).

The smallest antigen binding fragment is the single variable fragment, namely the variable heavy (V_{H}) or variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “immunoglobulin single variable domain”. A single domain antibody (˜12 to 15 kDa) has thus either the V_{H} or V_{L} domain.

Thus, in one embodiment, the fragment is selected from a F(ab′)2, Fab, Fv, scFv, heavy chain, light chain, variable heavy (V_{H}), variable light (V_{L}) chain, CDR region, single V_{H} or V_{L} domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

In one embodiment, the invention does not relate to an immunoglobulin domain, e.g. an Fc domain, fused to a companion animal, e.g. canine, OX40L extracellular domain polypeptide fragment or biological equivalent thereof.

The antibodies and antibody fragments of the invention are isolated. The term “isolated” as used herein refers to a moiety that is isolated from its natural environment. For example, the term “isolated” refers to a single domain antibody that is substantially free of other single domain antibodies, antibodies or antibody fragments. Moreover, an isolated single domain antibody may be substantially free of other cellular material and/or chemicals.

In one aspect, the invention relates to an antibody or fragment thereof that binds specifically to companion animal, such as canine, OX40L wherein said antibody blocks binding of OX40L to OX40 and/or inhibits one or more functions associated with binding of OX40L to OX40. Suitably the antibody reduces, inhibits or neutralises OX40 activity in the companion animal. In one embodiment, the antibody or fragment thereof exhibits one or more of the following properties:
a) Is capable of modifying secretion of a cytokine in the cell or animal, and/or
b) Is capable of decreasing proliferation of leukocytes in the companion animal, such as dog.

“Modifying” refers to increasing or decreasing the amount of a compound in the presence of an antibody compared to a control. “Decreasing” or “decreases” as used herein refers to a reduction and the decrease may be at least 10%, 20%, 30%, 40%, 50%, 60%, 70%, 80%, 90%, 95% or more.

For example, the antibody or fragment is capable of
a) Decreasing secretion of inflammatory cytokine in the companion animal or in a cell of the companion animal and/or
b) Decreasing secretion of an inflammatory chemokine or chemokine receptor in the companion animal or in a cell of the companion animal and/or
c) Increasing the secretion of suppressive cytokine(s) in the companion animal or in a cell of the companion animal and/or
d) Increasing the secretion of suppressive chemokines(s) or chemokine receptors in the companion animal or in a cell of the companion animal and/or
e) Decreasing proliferation of leukocytes in the companion animal.

The cytokine may be selected from TNF alpha, IL-2, IL-3, IL-4, IL-5, IL-6, IL-8, IL-9, IL-10, IL-13, IL-17, RANTES, GM-CSF, TGF-β and interferon gamma.

An “inflammatory” compound is one that is involved in promoting inflammation, whereas a “suppressive” compound is one that is involved in suppressing or regulating inflammation. Inflammatory cytokines include interleukin-1 (IL-1), IL-12, and IL-18, TNF alpha, interferon gamma (IFNγ) and GM-CSF. Suppressive or anti-inflammatory cytokines or receptors include IL-4, IL-10, IL-11, IL-13 and TGF-β. The cytokine may be a chemokine. In one embodiment, the chemokine may be selected from CXCL13, CXCR5, for example. The antibody in accordance with the invention may also modify cytokine or chemokine receptor expression.

Assays may be carried out in vitro (e.g. using a cell, cells or tissue) or in vivo.

In one embodiment, binding of OX40L to OX40 in the presence of an antibody in accordance with the invention may be determined in an SPR (surface plasmon resonance) assay, for example. Other methods for determining inhibition of an OX40L/OX40 interaction are described, for example in WO2016/139482 or WO2013/008171 and include, for example, flow cytometry monitoring of antibody binding to recombinant OX40L-expressing cells.

In one embodiment, the ability of an antibody to OX40L to block binding of OX40L to OX40 can be measured by measuring an inhibition of NFkB activity. Suitable assays for measuring NFkB activity include the HEK-blue assay described herein. Accordingly, in one embodiment, the antibody or fragment thereof that binds specifically to companion animal, such as canine, OX40L reduces, inhibits or neutralises OX40R-mediated NFkB activity in a cell-based assay.

In one embodiment, the assay is a heterologous assay in which a companion animal (e.g. dog) OX40 is used in a cell line derived from a different species, e.g. a human cell line such as HEK, substantially as described in Example 7 herein.

In one embodiment, the ability of an antibody to OX40L to block binding of OX40L to OX40 can be measured by measuring a decreased secretion of a cytokine in a cell compared to that observed in the absence of the antibody. In one embodiment, the ability of an antibody to OX40L to block binding of OX40L to OX40 can be measured by measuring an inhibition of IL-2 or INF-gamma secretion from PBMCs. Accordingly, in one embodiment, the antibody or fragment thereof that binds specifically to companion animal, such as canine, OX40L reduces, inhibits or neutralises OX40R-mediated IL-2 or INF-gamma (INFγ) secretion from PBMCs. In another embodiment, the ability of an antibody to OX40L to block binding of OX40L to OX40 can be measured by measuring an inhibition of IL-13 secretion from PBMCs. It will be understood that the ability of an antibody to OX40 to block binding of OX40L to OX40 can be measured in a similar manner.

The antibody or fragment is capable of effecting a decrease of the proliferation of leukocytes (e.g., mononuclear cells) in an in vitro assay wherein the antibody or fragment antagonises OX40L/OX40L receptor interaction.

As is known in the art, the term “leukocytes” includes, for example, one or more of lymphocytes, polymorphonuclear leukocyte and monocytes. As is also readily apparent to the skilled person the term “monocytes” includes, for example, peripheral blood mononuclear cells (PBMCs) or monocyte derived cells, e.g., dendritic cells (DCs).

Leukocyte proliferation may be measured, for example in a Mixed Lymphocyte Reaction (MLR) as described herein. The ability of an antibody in accordance with the invention to decrease proliferation may be measured by comparison to proliferation in the absence of the antibody.

The proliferation of leukocytes, e.g., lamina propria lymphocytes (LPLs), can be assessed using tissue biopsy, staining and histology, as will be apparent to the skilled person. Hematoxylin and eosin stain (H&E stain or HE stain) is, for example, commonly used in histology to look for infiltrating lymphocytes a whole range of human tissue and is one of the principal stains in histology. It is the most widely used stain in medical diagnosis and is often the gold standard, and as such can be used to assess proliferation of leukocytes as per the invention. For example, GI tract tissue (e.g., gut tissue) from a companion animal that is suffering from or at risk of a OX40L-mediated disease or condition can be obtained, stained and assessed for the extent of infiltration of LPLs.

Comparison can be made between such tissue from a companion animal that has received an antibody of the invention compared to the extent of infiltration in tissue obtained from the same animal prior to administration of antibody or from another companion animal that has not received treatment and is at risk of or suffering from the disease or condition. For example, the comparison is between companion animal gut tissues taken from the same (or different) companion animals suffering from e.g. IBD. The anti-OX40L antibody binds to OX40L and regulates cytokine and cellular receptor expression resulting in cytokine levels characteristic of non-disease states. The anti-OX40 antibody binds to OX40 and regulates cytokine and cellular receptor expression resulting in cytokine levels characteristic of non-disease states.

Cytokines are indispensable signals of the mucosa-associated immune system for maintaining normal gut homeostasis. An imbalance of their profile in favour of inflammation initiation may lead to disease states, such as that is observed in inflammatory bowel diseases (IBD), e.g., Crohn's disease (CD) and ulcerative colitis (UC). The role of pro-inflammatory cytokines such as IL-Ia, IL-Iβ, IL-2, -6, -8, -12, -17, -23, IFN-gamma, or TNF alpha in IBD is associated with the initiation and progression of UC and CD. CD is often described as a prototype of T-helper (Th) 1-mediated diseases because the primary inflammatory mediators are the Thl cytokines such as interleukin (IL)-12, interferon (IFN)-y, and tumour necrosis factor (TNF)-α. The cytokine or cytokine receptor may be selected from TNF alpha, IL-2, IL-3, IL-4, IL-5, IL-6, IL-8, IL-9, IL-10, IL-13, IL-17, RANTES, GM-CSF, TGF-β and interferon gamma.

Further information on suitable assays to assess properties of the antibodies is provided in the examples.

In one embodiment, the antibody that binds canine OX40L may be selected from one of the following antibodies:

An antibody comprising a HC CDR1 comprising SEQ ID No: 15 or a sequence with at least 80% sequence identity thereto, a HC CDR2 comprising SEQ ID No: 16 or a sequence with at least 80% sequence identity thereto, a HC CDR3 comprising SEQ ID No: 17 or a sequence with at least 80% sequence identity thereto, a LC CDR1 comprising SEQ ID No: 18 or a sequence with at least 80% sequence identity thereto, a LC CDR2 comprising SEQ ID No: 19 or a sequence with at least 80% sequence identity thereto and a LC CDR3 comprising SEQ ID No: 20 or a sequence with at least 80% sequence identity thereto;

An antibody comprising a HC CDR1 comprising SEQ ID No: 25 or a sequence with at least 80% sequence identity thereto, a HC CDR2 comprising SEQ ID No: 26 or a sequence with at least 80% sequence identity thereto, a HC CDR3 comprising SEQ ID No: 27 or a sequence with at least 80% sequence identity thereto, a LC CDR1 comprising SEQ ID No: 28 or a sequence with at least 80% sequence identity thereto, a LC CDR2 comprising SEQ ID No: 29 or a sequence with at least 80% sequence identity thereto and a LC CDR3 comprising SEQ ID No: 30 or a sequence with at least 80% sequence identity thereto;

An antibody comprising a HC CDR1 comprising SEQ ID No: 35 or a sequence with at least 80% sequence identity thereto, a HC CDR2 comprising SEQ ID No: 36 or a sequence with at least 80% sequence identity thereto, a HC CDR3 comprising SEQ ID No: 37 or a sequence with at least 80% sequence identity thereto, a LC CDR1 comprising SEQ ID No: 38 or a sequence with at least 80% sequence identity thereto, a LC CDR2 comprising SEQ ID No: 39 or a sequence with at least 80% sequence identity thereto and a LC CDR3 comprising SEQ ID No: 40 or a sequence with at least 80% sequence identity thereto;

An antibody comprising a HC CDR1 comprising SEQ ID No: 45 or a sequence with at least 80% sequence identity thereto, a HC CDR2 comprising SEQ ID No: 46 or a sequence with at least 80% sequence identity thereto, a HC CDR3 comprising SEQ ID No: 47 or a sequence with at least 80% sequence identity thereto, a LC CDR1 comprising SEQ ID No: 48 or a sequence with at least 80% sequence identity thereto, a LC CDR2 comprising SEQ ID No: 49 or a sequence with at least 80% sequence identity thereto and a LC CDR3 comprising SEQ ID No: 50 or a sequence with at least 80% sequence identity thereto;

An antibody comprising a HC CDR1 comprising SEQ ID No: 55 or a sequence with at least 80% sequence identity thereto, a HC CDR2 comprising SEQ ID No: 56 or a sequence with at least 80% sequence identity thereto, a HC CDR3 comprising SEQ ID No: 57, or a sequence with at least 80% sequence identity thereto, a LC CDR1 comprising SEQ ID No: 58 or a sequence with at least 80% sequence identity thereto, a LC CDR2 comprising SEQ ID No: 59 or a sequence with at least 80% sequence identity thereto and a LC CDR3 comprising SEQ ID No: 60 or a sequence with at least 80% sequence identity thereto;

An antibody comprising a HC CDR1 comprising SEQ ID No: 65 or a sequence with at least 80% sequence identity thereto, a HC CDR2 comprising SEQ ID No: 66 or a sequence with at least 80% sequence identity thereto, a HC CDR3 comprising SEQ ID No: 67 or a sequence with at least 80% sequence identity thereto, a LC CDR1 comprising SEQ ID No: 68 or a sequence with at least 80% sequence identity thereto, a LC CDR2 comprising SEQ ID No: 69 or a sequence with at least 80% sequence identity thereto and a LC CDR3 comprising SEQ ID No: 70 or a sequence with at least 80% sequence identity thereto;

An antibody comprising a HC CDR1 comprising SEQ ID No: 75 or a sequence with at least 80% sequence identity thereto, a HC CDR2 comprising SEQ ID No: 76 or a sequence with at least 80% sequence identity thereto, a HC CDR3 comprising SEQ ID No: 77 or a sequence with at least 80% sequence identity thereto, a LC CDR1 comprising SEQ ID No: 78 or a sequence with at least 80% sequence identity thereto, a LC CDR2 comprising SEQ ID No: 79 or a sequence with at least 80% sequence identity thereto and a LC CDR3 comprising SEQ ID No: 80 or a sequence with at least 80% sequence identity thereto;

An antibody comprising a HC CDR1 comprising SEQ ID No: 85 or a sequence with at least 80% sequence identity thereto, a HC CDR2 comprising SEQ ID No: 86 or a sequence with at least 80% sequence identity thereto, a HC CDR3 comprising SEQ ID No: 87 or a sequence with at least 80% sequence identity thereto, a LC CDR1 comprising SEQ ID No: 88 or a sequence with at least 80% sequence identity thereto, a LC CDR2 comprising SEQ ID No: 89 or a sequence with at least 80% sequence identity thereto and a LC CDR3 comprising SEQ ID No: 90 or a sequence with at least 80% sequence identity thereto;

An antibody comprising a HC CDR1 comprising SEQ ID No: 95 or a sequence with at least 80% sequence identity thereto, a HC CDR2 comprising SEQ ID No: 96 or a sequence with at least 80% sequence identity thereto, a HC CDR3 comprising SEQ ID No: 97 or a sequence with at least 80% sequence identity thereto, a LC CDR1 comprising SEQ ID No: 98 or a sequence with at least 80% sequence identity thereto, a LC CDR2 comprising SEQ ID No: 99 or a sequence with at least 80% sequence identity thereto and a LC CDR3 comprising SEQ ID No: 100 or a sequence with at least 80% sequence identity thereto;

An antibody comprising a HC CDR1 comprising SEQ ID No: 105 or a sequence with at least 80% sequence identity thereto, a HC CDR2 comprising SEQ ID No: 106 or a sequence with at least 80% sequence identity thereto, a HC CDR3 comprising SEQ ID No: 107 or a sequence with at least 80% sequence identity thereto, a LC CDR1 comprising SEQ ID No: 108 or a sequence with at least 80% sequence identity thereto, a LC CDR2 comprising SEQ ID No: 109 or a sequence with at least 80% sequence identity thereto and a LC CDR3 comprising SEQ ID No: 110 or a sequence with at least 80% sequence identity thereto;

An antibody comprising a HC CDR1 comprising SEQ ID No: 115 or a sequence with at least 80% sequence identity thereto, a HC CDR2 comprising SEQ ID No: 116 or a sequence with at least 80% sequence identity thereto, a HC CDR3 comprising SEQ ID No: 117 or a sequence with at least 80% sequence identity thereto, a LC CDR1 comprising SEQ ID No: 118 or a sequence with at least 80% sequence identity thereto, a LC CDR2 comprising SEQ ID No: 119 or a sequence with at least 80% sequence identity thereto and a LC CDR3 comprising SEQ ID No: 120 or a sequence with at least 80% sequence identity thereto;

An antibody comprising a HC CDR1 comprising SEQ ID No: 125 or a sequence with at least 80% sequence identity thereto, a HC CDR2 comprising SEQ ID No: 126 or a sequence with at least 80% sequence identity thereto, a HC CDR3 comprising SEQ ID No: 127 or a sequence with at least 80% sequence identity thereto, a LC CDR1 comprising SEQ ID No: 128 or a sequence with at least 80% sequence identity thereto, a LC CDR2 comprising SEQ ID No: 129 or a sequence with at least 80% sequence identity thereto and a LC CDR3 comprising SEQ ID No: 130 or a sequence with at least 80% sequence identity thereto; or

An antibody comprising a HC CDR1 comprising SEQ ID No: 135 or a sequence with at least 80% sequence identity thereto, a HC CDR2 comprising SEQ ID No: 136 or a sequence with at least 80% sequence identity thereto, a HC CDR3 comprising SEQ ID No: 137 or a sequence with at least 80% sequence identity thereto, a LC CDR1 comprising SEQ ID No: 138 or a sequence with at least 80% sequence identity thereto, a LC CDR2 comprising SEQ ID No: 139 or a sequence with at least 80% sequence identity thereto and a LC CDR3 comprising SEQ ID No: 140 or a sequence with at least 80% sequence identity thereto.

In one embodiment the antibody is selected from one of those recited above, but comprises one or more HC and/or LC CDR1, 2 and/or CDR3 sequences with 1, 2, 3, 4 or 5 amino acid substitutions compared to the sequences defined in the SEQ ID Nos. above.

Fragments of the antibodies listed above are also provided and within the scope of the invention.

In one embodiment, the antibody may be selected from one of the following antibodies:

An antibody comprising a HC variable region comprising or consisting of SEQ ID No. 12 or a sequence with at least 80% sequence identity thereto and a LC variable region comprising or consisting of SEQ ID No. 14 or a sequence with at least 80% sequence identity thereto;

An antibody comprising a HC variable region comprising or consisting of SEQ ID No. 22 or a sequence with at least 80% sequence identity thereto and a LC variable region comprising or consisting of SEQ ID No. 24 or a sequence with at least 80% sequence identity thereto;

An antibody comprising a HC variable region comprising or consisting of SEQ ID No. 32 or a sequence with at least 80% sequence identity thereto and a LC variable region comprising or consisting of SEQ ID No. 34 or a sequence with at least 80% sequence identity thereto;

An antibody comprising a HC variable region comprising or consisting of SEQ ID No. 42 or a sequence with at least 80% sequence identity thereto and a LC variable region comprising or consisting of SEQ ID No. 44 or a sequence with at least 80% sequence identity thereto;

An antibody comprising a HC variable region comprising or consisting of SEQ ID No. 52 or a sequence with at least 80% sequence identity thereto and a LC variable region comprising or consisting of SEQ ID No. 54 or a sequence with at least 80% sequence identity thereto;

An antibody comprising a HC variable region comprising or consisting of SEQ ID No. 62 or a sequence with at least 80% sequence identity thereto and a LC variable region comprising or consisting of SEQ ID No. 64 or a sequence with at least 80% sequence identity thereto;

An antibody comprising a HC variable region comprising or consisting of SEQ ID No. 72 or a sequence with at least 80% sequence identity thereto and a LC variable region comprising or consisting of SEQ ID No. 74 or a sequence with at least 80% sequence identity thereto;

An antibody comprising a HC variable region comprising or consisting of SEQ ID No. 82 or a sequence with at least 80% sequence identity thereto and a LC variable region comprising or consisting of SEQ ID No. 84 or a sequence with at least 80% sequence identity thereto;

An antibody comprising a HC variable region comprising or consisting of SEQ ID No. 92 or a sequence with at least 80% sequence identity thereto and a LC variable region comprising or consisting of SEQ ID No. 94 or a sequence with at least 80% sequence identity thereto;

An antibody comprising a HC variable region comprising or consisting of SEQ ID No. 102 or a sequence with at least 80% sequence identity thereto and a LC variable region comprising or consisting of SEQ ID No. 104 or a sequence with at least 80% sequence identity thereto;

An antibody comprising a HC variable region comprising or consisting of SEQ ID No. 112 or a sequence with at least 80% sequence identity thereto and a LC variable region comprising or consisting of SEQ ID No. 114 or a sequence with at least 80% sequence identity thereto;

An antibody comprising a HC variable region comprising or consisting of SEQ ID No. 122 or a sequence with at least 80% sequence identity thereto and a LC variable region comprising or consisting of SEQ ID No. 124 or a sequence with at least 80% sequence identity thereto; or

An antibody comprising a HC variable region comprising or consisting of SEQ ID No. 132 or a sequence with at least 80% sequence identity thereto and a LC variable region comprising or consisting of SEQ ID No. 134 or a sequence with at least 80% sequence identity thereto.

In one embodiment the antibody is selected from one of those recited above and comprises HC variable region and/or LC variable region which comprises 1 to 20, e.g. 1 to 10, e.g., 2, 3, 4 or 5 amino acid substitutions compared to the sequences as defined by the SEQ ID Nos above.

Fragments of the antibodies listed above are also provided.

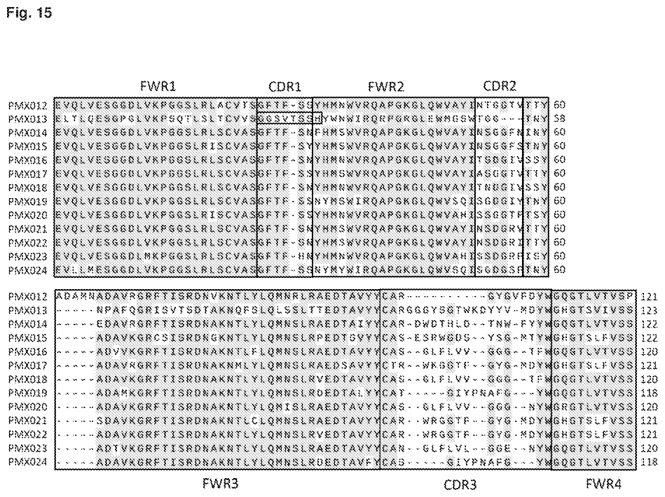

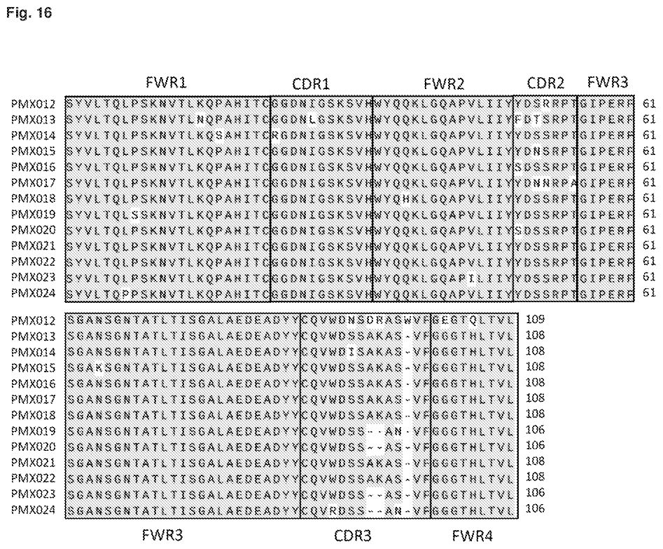

The amino acid sequences of the VH and VL regions of the antibodies are shown in FIGS. 15 and 16. An antibody or fragment may therefore be selected from an antibody comprising or consisting of a sequence shown in these figures. In one embodiment, the antibody is selected from PMX012, PMX013, PMX014, PMX015, PMX016, PMX017, PMX018, PMX019, PMX020, PMX021, PMX022, PMX023 or PMX024. In one embodiment, the antibody is PMX023 or PMX012. Sequences are also shown in table 2.

FIGS. 17 and 18 show that the VH and VL regions of the antibodies can be grouped into different groups, i.e. families based on sequence comparison and each family shares significant sequence identity. Group A includes PMX016, PMX018, PMX020 and PMX023. Group B includes PMX017, PMX021, PMX 022. Group C includes PMX019 and PMX 024. The other antibodies described herein do not fall into these families. In one embodiment, the antibody or fragment may be selected from an antibody as shown in Group A or an antibody with at least 80% sequence identity thereto, an antibody as shown in Group B or an antibody with at least 80% sequence identity thereto or an antibody as shown in Group C or an antibody with at least 80% sequence identity thereto.

All sequence for antibody and antibody fragments designated as PMX as shown in the figures herein and in table 2 are within the scope of the invention.

Sequence identity as described in the various embodiments above is at least 80%. In one embodiment, sequence identity is at least 79%, 80%, 81%, 82%, 83%, 84%, 85%, 86%, 87%, 88%, 89%, 90%, 91%, 92%, 93%, 94%, 95%, 96%, 97%, 98% or 99%. In one embodiment, said sequence identity is at least 90%, 91%, 92%, 93%, 94%, 95%, 96%, 97%, 98% or 99%.

As described above, the antibody or fragment defined by reference to the sequence may comprise one or more amino acid substitutions. In one embodiment, the modification is a conservative sequence modification. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within one or more the CDR region and/or one or more framework region the antibody or fragment of the invention can be replaced with other amino acid residues from the same side chain family and the altered antibody can be tested for retained function (using the functional assays described herein or known in the art.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues of the polypeptides and peptides described herein, and conservative substitutions for these amino acid residues are shown in Table 1 below.

TABLE 1
Amino Acid Residues and Examples of
Conservative Amino Acid Substitutions

| Original residue | |
|---|---|
| Three letter code, single letter code | Conservative substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidein, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

Antibodies described herein may comprise suitable Fc regions. In one embodiment, the antibody or antigen-binding portion thereof comprises an Fc region, for example a canine Fc region, for example a canine IgGB Fc region. In one embodiment, the Fc portion of the antibody may be modified. For example, modifications may be made to the Fc region to improve certain properties, e.g. to provide reduced complement- and FcγR-mediated effector functions.

Exemplary modified Fc regions for canine antibodies of the invention based on a canine IgG-B Fc region are provided in SEQ ID Nos 142 to 150 which show the IgG-B constant region, including the Fc region. These sequences comprise modifications compared to wild type Fc IgG-B regions. The modified Fc regions reduce or abolish canine IgG-B effector function when compared to the same polypeptide comprising a wild-type IgG-B Fc domain. This is shown in the examples. The amino acid substitutions reside in the lower hinge, proline sandwich region and SHED region. Thus, an antibody of the invention may include a modified Fc region having the modifications as shown in SEQ ID Nos 142 to 150. Modifications are with reference to the wt sequence as shown in SEQ ID NO. 141.

Thus, with reference to the wild type residue in canine IgG-B constant region (SEQ ID NO: 141), the antibodies may have the following amino acid substitutions at the following positions in the Fc domain:
E119G;
M120S or A;
L121A;
D153G;
P154R;
D156N;
N211H;
K212I;
A213G;
P215G and/or
P217S.

In one embodiment, the Fc region comprises
a) an amino acid substitution at position 120 of SEQ ID NO: 141 to S and
b) an amino acid substitution at position 211 of SEQ ID NO: 141 to H, an amino acid substitution at position 212 of SEQ ID NO: 141 to I and an amino acid substitution at position 213 of SEQ ID NO: 141 to G.

In one embodiment, the Fc region comprises
a) an amino acid substitution at position 120 of SEQ ID NO: 141 to S;
b) an amino acid substitution at position 153 of SEQ ID NO: 141 to G and an amino acid substitution at position 154 of SEQ ID NO: 141 to R and
c) an amino acid substitution at position 211 of SEQ ID NO: 141 to H, an amino acid substitution at position 212 of SEQ ID NO: 141 to I and an amino acid substitution at position 213 of SEQ ID NO: 141 to G.

In one embodiment, the Fc region comprises
a) an amino acid substitution at position 120 of SEQ ID NO: 141 to S;
b) an amino acid substitution at position 153 of SEQ ID NO: 141 to G and an amino acid substitution at position 154 of SEQ ID NO: 141 to R and
c) an amino acid substitution at position 211 of SEQ ID NO: 141 to H, an amino acid substitution at position 212 of SEQ ID NO: 141 to I and an amino acid substitution at position 213 of SEQ ID NO: 141 to G and an amino acid substitution at position 217 of SEQ ID NO: 141 to S.

In one embodiment, the Fc region comprises
a) an amino acid substitution at position 120 of SEQ ID NO: 141 to S;
b) an amino acid substitution at position 153 of SEQ ID NO: 141 to G and an amino acid substitution at position 154 of SEQ ID NO: 141 to R and
c) an amino acid substitution at position 211 of SEQ ID NO: 141 to H, an amino acid substitution at position 212 of SEQ ID NO: 141 to I and an amino acid substitution at position 213 of SEQ ID NO: 141 to G and an amino acid substitution at position 215 of SEQ ID NO: 141 to G.

In one embodiment, the Fc region comprises an amino acid substitution at position 120 of SEQ ID NO: 141 to A and at position 121 to A.

In one embodiment, the Fc region comprises
a) an amino acid substitution at position 120 of SEQ ID NO: 141 to A and at position 121 to A and
b) an amino acid substitution at position 217 of SEQ ID NO: 141 to S.

In one embodiment, the Fc region comprises
a) an amino acid substitution at position 120 of SEQ ID NO: 141 to A and at position 121 to A and
b) an amino acid substitution at position 215 of SEQ ID NO: 141 to G.

In one embodiment, the Fc region comprises
a) an amino acid substitution at position 119 of SEQ ID NO: 141 to G and
b) an amino acid substitution at position 156 of SEQ ID NO: 141 to N.

In one embodiment, the Fc region comprises
a) an amino acid substitution at position 120 of SEQ ID NO: 141 to A and at position 121 to A;
b) an amino acid substitution at position 156 of SEQ ID NO: 141 to N and
c) an amino acid substitution at position 217 of SEQ ID NO: 141 to S.

In another aspect, there are provided binding molecules, e.g. antibodies, antibody fragments or antibody mimetics that bind at or near the same epitope or an overlapping epitope on the companion animal OX40L or OX40 respectively (e.g. dog OX40 or OX40L) as any of the OX40L/OX40 antibodies of the invention (i.e., antibodies that have the ability to cross-compete for binding to OX40L/OX40 with an antibody of the invention). The antibodies of the invention can thus be used as a reference antibody.

Such cross-competing antibodies can be identified based on their ability to cross-compete with an antibody described herein in standard OX40L/OX40 binding assays. For example, SPR analysis such as BIAcore® analysis, BLI analysis such as FortBio Octet®, ELISA assays or flow cytometry may be used to demonstrate cross-competition with the antibodies.

In one embodiment, there is provided a binding agent capable of binding companion animal OX40L or OX40 respectively (e.g. dog OX40 or OX40L) wherein an antibody of the invention displaces the binding agent in a competitive assay.

Included within the scope of this invention are antibody derivatives. A “derivative” of an antibody contains additional chemical moieties not normally a part of the protein. Covalent modifications of the protein are included within the scope of this invention. Such modifications may be introduced into the molecule by reacting targeted amino acid residues of the antibody with an organic derivatizing agent that is capable of reacting with selected side chains or terminal residues. For example, derivatization with bifunctional agents, well-known in the art, is useful for cross-linking the antibody or fragment to a water-insoluble support matrix or to other macromolecular carriers.

The antibody or fragment thereof as described herein can be used as a building block in a multispecific, for example bispecific or trispecific, binding agent that provides dual targeting of a companion animal OX40L or OX40 respectively (e.g. dog OX40 or OX40L) expressing cell. Thus, the antibody or fragment thereof as described herein is linked to another therapeutic entity that targets a different antigen. This other therapeutic entity is for example selected from an antibody or antibody fragment (e.g., a Fab, F(ab′)2, Fv, a single chain Fv fragment (scFv) or single domain antibody, for example a VH or VHH domain), CDR region or antibody mimetic protein. Suitable non-immunogenic linker peptides are known in the art, for example, linkers that include G and/or S residues, (G4S)n, (SG4)n or G4(SG4)n peptide linkers, wherein “n” is generally a number between 1 and 10.

In another embodiment, the antibody or fragment thereof according to the invention is linked to a further moiety that may serve to prolong the half-life of the molecule. The further moiety may comprise a protein, for example an antibody, or part thereof that binds a serum albumin, e.g., dog or cat serum albumin. Other modifications that prolong half-life are also known and include, for example, modification by PEG or by incorporation in a liposome.

In one embodiment, the antibody or fragment thereof according to the invention is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

In still other embodiments, the antibody or fragment thereof according to the invention is coupled to a toxin. In one embodiment, the therapeutic moiety is a toxin, for example a cytotoxic radionuclide, chemical toxin or protein toxin.

The term “half-life” as used herein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. Half-life may be increased by at least 1.5 times, preferably at least 2 times, such as at least 5 times, for example at least 10 times or more than 20 times, greater than the half-life of the corresponding antibodies of the invention. For example, increased half-life may be more than 1 hours, preferably more than 2 hours, more preferably more than 6 hours, such as more than 12 hours, or even more than 24, 48 or 72 hours, compared to the corresponding antibodies of the invention. The in vivo half-life of an amino acid sequence, compound or polypeptide of the invention can be determined in any manner known per se, such as by pharmacokinetic analysis. Suitable techniques will be clear to the person skilled in the art. Half-life can for example be expressed using parameters such as the t½-alpha t½-beta and the area under the curve (AUC).

The antibodies and fragments of the invention may also be used in cell therapy, for example chimeric antigen receptor T-cell (CAR-T) therapy.

Nucleic Acids

In another aspect, the invention relates to a nucleic acid sequence encoding an amino acid sequence of an antibody or antibody fragment as described herein.

In one embodiment, the nucleic acid encodes a HC variable region which comprises or consists of a sequence selected from SEQ ID No. 11, SEQ ID No. 21, SEQ ID No. 31, SEQ ID No. 41, SEQ ID No. 51, SEQ ID No. 61, SEQ ID No. 71, SEQ ID No. 81, SEQ ID No. 91, SEQ ID No. 101, SEQ ID No. 111, SEQ ID No. 121 or SEQ ID No. 131.

In one embodiment, the nucleic acid encodes a LC variable region which comprises or consists of a sequence selected from SEQ ID No. 13, SEQ ID No. 23, SEQ ID No. 33, SEQ ID No. 43, SEQ ID No. 53, SEQ ID No. 63, SEQ ID No. 73, SEQ ID No. 83, SEQ ID No. 93, SEQ ID No. 103, SEQ ID No. 113, SEQ ID No. 123 or SEQ ID No. 133.

Exemplary Methods for Making the Antibody

An antibody or fragment described herein can be obtained from a mammal, for example a rodent, for example a transgenic animal, that expresses antibodies upon stimulation with an OX40L or OX40 antigen of the target companion animal, e.g. dog or cat OX40L or OX40. Suitably companion animal antibody genes have been introduced such that companion animal antibodies are generated. The transgenic rodent, for example a mouse, preferably has a reduced capacity to express endogenous antibody genes. Thus, in one embodiment, the rodent has a reduced capacity to express endogenous light and/or heavy chain antibody genes. The rodent, for example a mouse, may therefore comprise modifications to disrupt expression of endogenous kappa and lambda light and/or heavy chain antibody genes so that no functional mouse light and/or heavy chains are produced, for example as further explained below. Such transgenic rodents are described in the art and this is further explained in the examples below.

Other methods may involve speciation of a mouse monoclonal typically following the steps of immunising a mouse with an OX40L or OX40 antigen of the target companion animal, isolating B cells and fusing them to a fusion partner cell line, isolating mouse monoclonal antibodies by selection. Speciation is then carried out by chimerization and/or further informatic-guided speciation. Strategies for speciation, such as strategies for caninization or felinization, are described, for example, in WO2013/011407, see Example 5.

In other methods, a set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty).

It is also possible to generate libraries, for example phage libraries, by isolating a cell or tissue expressing an antigen-specific, heavy chain-only antibody, cloning the sequence encoding the VH domain(s) from mRNA derived from the isolated cell or tissue and displaying the encoded protein using a library. The antibody or fragment can be expressed in bacterial, yeast or other expression systems.

Exemplary Therapeutic Applications

In one aspect, we provide an antibody or fragment as described herein for use in the treatment of disease.

In another aspect, there is provided a pharmaceutical composition comprising an antibody or fragment as described herein and optionally a pharmaceutically acceptable carrier. The term pharmaceutical composition as used herein refers to a composition that is used to treat a companion animal, that is for veterinary use, i.e. a veterinary composition. In preferred embodiments the animal that is treated is a dog or a cat.

An antibody or fragment thereof as described herein or the pharmaceutical composition of the invention can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

In one embodiment, administration is subcutaneous. The concentration of the antibody or antibody fragment may be 10 to 50 mg/ml, e.g. 10, 20, 30, 40 or 50 mg/ml. In one embodiment, the concentration is 25 to 35 mg/ml, for example about 30 mg/ml. In one embodiment, the antibody is provided as a dose in 1 ml of solution.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antibody or fragment thereof of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or subcutaneously.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by injection, intravenous injection or infusion.

The amount of the therapeutic that is effective/active in the treatment of a particular disorder or condition will depend on the nature of the disorder or condition and the animal to be treated and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each subject's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody of the present invention by weight of the composition. When intended for oral administration, this amount can be varied to range from about 0.1% to about 80% by weight of the composition. Preferred oral compositions can comprise from about 4% to about 50% of the antibody of the present invention by weight of the composition.

Preferred compositions of the present invention are prepared so that a parenteral dosage unit contains from about 0.01% to about 2% by weight of the antibody of the present invention.

For administration by injection, the composition can comprise from about typically about 0.1 mg/kg to about 250 mg/kg of the subject's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, 4, 5, 6, 7, 8 or more weeks.

In one embodiment, post-treatment, the subject has at least 7 days, or at least 14 days, or at least 21 days, or at least 28 days, or at least 40 days, or at least 50 days, or at least 60 days disease progression-free.

In one embodiment, the number of days of survival, the number of disease-free days, or the number of disease-progression free days is at least 2 months, or at least 3 months, or at least 4 months, e.g. at least 5 months, such as at least 6 months.

In one embodiment, the number of days of survival, the number of disease-free days, or the number of disease-progression free days is at least 9 months, or at least one year. The invention provides methods of treating or preventing OX40L/OX40-mediated diseases or disorders in a companion animal, e.g., a dog, cat or horse, comprising administering an effective amount of an antibody or fragment of the present invention to the animal in need thereof.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., companion animal patients, being treated. Many medical treatments are effective for some, but not all, patients that undergo the treatment.

Suitably, for treating a condition such as atopic dermatitis, post-treatment, a reduction in pruritus score is observed. Suitable methods for measuring pruritus score will be known to those skilled in the art.

In one embodiment, an antagonistic antibody in accordance with the invention, or fragment thereof, suppresses a GVHD reaction in a xenogenic graft versus host reaction such as described in WO2013/0008171.

The term “subject” or “patient” refers to a companion animal which is the object of treatment, observation, or experiment. By way of example only, a subject includes, but is not limited to, a dog, cat or horse. For the avoidance of doubt, the treatment of humans is excluded.

As used herein, the term “effective amount” means an amount of an anti-OX40L/OX40 antibody, that when administered alone or in combination with an additional therapeutic agent to a cell, tissue, or subject, is effective to achieve the desired therapeutic or prophylactic effect under the conditions of administration

In another aspect, the invention relates to the use of an antibody or fragment as described herein, or pharmaceutical composition of the invention in the manufacture of a medicament for the treatment or prevention of a disease as defined herein.

As mentioned above, the antibody or fragment according to the invention is useful in the treatment or prevention of an OX40L/OX40 mediated disease.

The term OX40L/OX40 mediated disease refers to any disease or disorder that is mediated by the OX40L/OX40 signalling pathway and which can be treated/alleviated by targeting the OX40L/OX40 antigen. An OX40L-mediated disease and OX40-mediated disease refer to any disease or condition that is completely or partially caused by or is the result of OX40L or OX40 respectively. In certain embodiments, OX40L or OX40 is aberrantly (e.g., highly) expressed on the surface of a cell. In some embodiments, OX40L or OX40 may be aberrantly upregulated on a particular cell type. In other embodiments, normal, aberrant or excessive cell signalling is caused by binding of OX40 to OX40L.

In one embodiment, the disease is an inflammatory condition which refers to pathological states resulting in inflammation or an autoimmune disease. In particular, the disease is selected from the following non-limiting list: inflammatory skin diseases, including atopic dermatitis (atopy), allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma; bacterial pneumonia, antigen-antibody complex mediated diseases; inflammations of the lung, including pleurisy, alveolitis, vasculitis, pneumonia, chronic bronchitis, bronchiectasis, and cystic fibrosis; etc. Other diseases include equine indications such as sweet itch, summer recurrent dermatitis or equine insect bite hypersensitivity.

Exemplary Combinations with Other Agents

The antibodies, antibody fragments or pharmaceutical composition of the invention may be administered as the sole active ingredient or in combination with one or more other therapeutic agent. A therapeutic agent is a compound or molecule which is useful in the treatment of a disease.

Examples of therapeutic agents include antibodies, antibody fragments, drugs, toxins, nucleases, hormones, an anti-inflammatory agent, immunomodulators, pro-apoptotic agents, anti-angiogenic agents, boron compounds, photoactive agents or dyes and radioisotopes. An antibody molecule includes a full antibody or fragment thereof (e.g., a Fab, F(ab′)2, Fv, a single chain Fv fragment (scFv) or a single domain antibody, for example a VH domain, or antibody mimetic protein.

In one embodiment, the antibody or antibody fragment or the pharmaceutical composition described herein is used in combination with an existing therapy or therapeutic agent. Thus, in another aspect, the invention also relates to a combination therapy comprising administration of the antibody or antibody fragment or the pharmaceutical composition described herein and another therapy.

In one embodiment, the therapeutic agent is selected from the following non-limiting list: rapamycin (sirolimus), tacrolimus, cyclosporin, corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, and PDE4 inhibitors lokietmab (Cytopoint®), cyclosporin (Atopica®) and oclacitinib (Apoquel®). In some embodiments, the antibody or antibody fragment or the pharmaceutical composition described herein may be administered with two or more therapeutic agents.

The antibody or antibody fragment or the pharmaceutical composition described herein may be administered at the same time or at a different time as the other therapy or therapeutic compound or therapy, e.g., simultaneously, separately or sequentially.

Exemplary Kits

In another aspect, the invention provides a kit for the treatment or prevention of a disease for example as listed herein and/or for detecting OX40L/OX40 for diagnosis, prognosis or monitoring disease comprising an antibody or fragment of the invention. Such a kit may contain other components, packaging, instructions, or material to aid in the detection of OX40L/OX40 protein. The kit may include a labelled antibody that binds to OX40L/OX40 or a binding molecule comprising an antibody that binds to OX40L/OX40 and one or more compounds for detecting the label.

The invention in another aspect provides an antibody or fragment thereof that binds to OX40L/OX40, or pharmaceutical composition described herein packaged in lyophilized form, or packaged in an aqueous medium.

Exemplary Non Therapeutic Applications

In another aspect, an antibody or fragment that binds to OX40L/OX40 as described herein is used for non-therapeutic purposes, such as diagnostic tests and assays. A method for detecting the presence of a companion animal OX40L/OX40 in a test sample comprises contacting said sample with an antibody or fragment thereof as described herein and at least one detectable label and detecting binding of said antibody to a companion animal OX40L/OX40.

Modifications of antibodies for diagnostic purposes are well known in the art. For example, antibodies may be modified with a ligand group such as biotin, or a detectable marker group such as a fluorescent group, a radioisotope, or an enzyme. Compounds of the invention can be used for diagnostic purposes and e.g. labelled using conventional techniques. Suitable detectable labels include but are not limited to fluorophores, chromophores, radioactive atoms, electron-dense reagents, enzymes, and ligands having specific binding partners.

In some embodiments, the binding of antigen to antibody is detected without the use of a solid support. For example, the binding of antigen to antibody can be detected in a liquid format.

In other embodiments, an antibody or fragment can, for example, be fixed to nitrocellulose, or another solid support which is capable of immobilizing cells, cell particles or soluble proteins. The support can then be washed with suitable buffers followed by treatment with the detectably labelled antibody. The solid phase support can then be washed with the buffer a second time to remove unbound peptide or antibody. The amount of bound label on the solid support can then be detected by known method steps. “Solid phase support” or “carrier” refers to any support capable of binding peptide, antigen, or antibody. Well-known supports or carriers, include glass, polystyrene, polypropylene, polyethylene, polyvinylidenefluoride (PVDF), dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration so long as the coupled molecule is capable of binding to OX40L/OX40 or an anti-OX40L/OX40 antibody. Thus, the support configuration can be spherical, as in a bead, or cylindrical, as in the inside surface of a test tube, or the external surface of a rod. Alternatively, the surface can be flat, such as a sheet, culture dish, test strip, etc. For example, supports may include polystyrene beads. Those skilled in the art will know many other suitable carriers for binding antibody, peptide or antigen, or can ascertain the same by routine experimentation. Well known method steps can determine binding activity of a given lot of anti-OX40L/OX40 antibody. Those skilled in the art can determine operative and optimal assay conditions by routine experimentation.

For the purposes of the present invention, the OX40L/OX40 which is detected by the above assays can be present in a biological test sample. Any sample containing OX40L/OX40 may be used. For example, the sample is a biological fluid such as, for example, blood, serum, lymph, urine, feces, inflammatory exudate, cerebrospinal fluid, amniotic fluid, a tissue extract or homogenate, and the like.

OX40 Protein and Nucleic Acid Sequences

The inventors have identified and isolated splice variants of OX40 in dogs.

In another aspect, the invention relates to an isolated canine OX40 amino acid sequence. The invention also relates to an isolated nucleic acid sequence encoding canine OX40 protein.

In one embodiment, the isolated canine OX40 protein comprises SEQ ID NO. 4 or 6 or a variant thereof.

In one embodiment, the isolated nucleic acid molecule encoding a protein as above, optionally comprises SEQ ID NO. 3 or 5 or a variant thereof.

A nucleic acid according to the present invention may comprise DNA or RNA and may be wholly or partially synthetic or recombinantly produced. Reference to a nucleotide sequence as set out herein encompasses a DNA molecule with the specified sequence, and encompasses an RNA molecule with the specified sequence in which U is substituted for T, unless context requires otherwise.

In another aspect, the invention relates to a trimeric soluble companion animal, e.g. canine OX40L extra cellular domain probe and its use in in a method of screening for companion animal, e.g. canine OX40L antibodies. The term trimeric refers to the conformation of OX40L extra cellular domain. Methods for screening a sample for the presence of companion animal, e.g. canine, OX40L antibodies are also within the scope. Such methods comprise exposing a sample to the probe and assessing the presence of OX40 antibodies. The sample may be any sample from the animal, e.g. a cell, tissue, blood, saliva or other suitable sample.

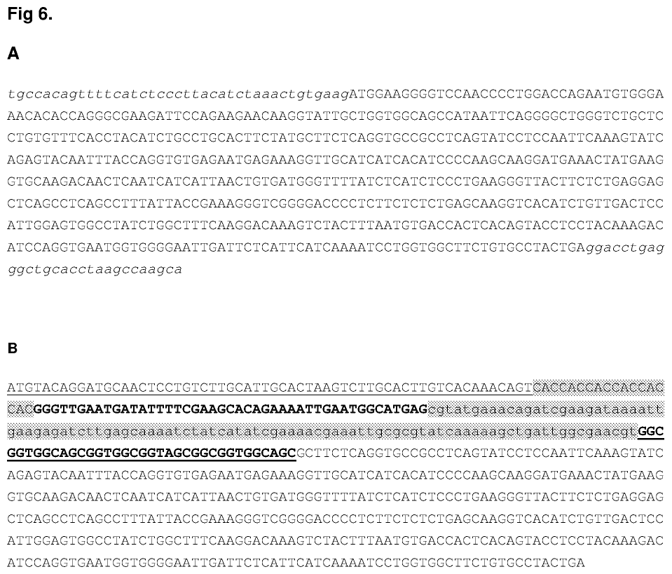

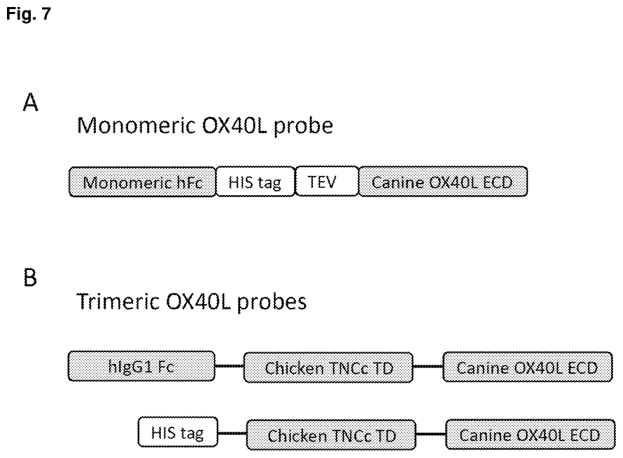

A trimeric soluble OX40L extra cellular domain probe is shown herein, e.g. in the schematic in FIG. 7. This contains chicken tenascin C trimerization domain and either human IgG1 Fc or HIS tags. Suitable sequence is also provided, see for example FIG. 6.

Nucleic Acid Construct and Hosts Cells

Furthermore, the invention relates to a nucleic acid construct comprising at least one nucleic acid as defined herein, i.e. nucleic acid molecules encoding antibodies of the invention or nucleic acid molecules encoding an OX40 protein. The construct may be in the form of a plasmid, vector, transcription or expression cassette.

An expression vector can be, for example, a plasmid, such as pBR322, pUC, or Col E1, or an adenovirus vector, such as an adenovirus Type 2 vector or Type 5 vector. Vectors suitable for use in the present invention include, for example, bacterial vectors, mammalian vectors, viral vectors (such as retroviral, adenoviral, adeno-associated viral, herpes virus, simian virus 40 (SV40), and bovine papilloma virus vectors) and baculovirus-derived vectors for use in insect cells. Polynucleotides in such vectors are preferably operably linked to a promoter, which is selected based on, e.g., the cell type in which expression is sought.

The expression vector can be transferred to a host cell by conventional techniques and the transfected cells are then cultured by conventional techniques to produce an antibody of the invention. The invention includes host cells containing polynucleotides encoding an antibody of the invention (e.g., whole antibody, a heavy or light chain thereof, or portion thereof, or a single chain antibody of the invention, or a fragment or variant thereof), operably linked to a heterologous promoter. For the expression of entire antibody molecules, vectors encoding both the heavy and light chains may be co-expressed in the host cell for expression of the entire immunoglobulin molecule. In one embodiment, the expression vector is one which enables heterologous expression e.g. expression in a host organism from different species.

The invention also relates to an isolated recombinant host cell comprising one or more nucleic acid construct as described herein, i.e. nucleic acid molecules encoding antibodies of the invention or nucleic acid molecules encoding an OX40 protein. Host cells useful in the present invention are prokaryotic, yeast, or higher eukaryotic cells and include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g. Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Prokaryotes useful as host cells in the present invention include gram negative or gram-positive organisms such as E. coli, B. subtilis, Enterobacter, Erwinia, Klebsiella, Proteus, Salmonella, Serratia, and Shigella, as well as Bacilli, Pseudomonas, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. In one embodiment, a method of making an anti-OX40 antibody as described herein is provided, wherein the method comprises culturing the host cell under conditions suitable for expression of the polynucleotide encoding the antibody and isolating the antibody.

In some embodiments, the nucleic acid may also comprise a leader sequence. Any suitable leader sequences may be used, including the native immunoglobulin germline leader sequence, or others, such as the Campath leader sequence (see U.S. Pat. No. 8,362,208B2), may be chosen to enhance protein expression.

Assays and Expression Systems

The invention also relates to a heterologous assay or expression system comprising a companion animal (e.g. dog) OX40 and a cell line derived from a different species, e.g. a human cell line such as HEK.

The assay comprises contacting a companion animal (e.g. dog) OX40 with a cell line derived from a different species, e.g. a cell line from a different mammal, e.g. a rodent cell line or a human cell line such as HEK. For example, the cell line is transfected with companion animal (e.g. dog) OX40 such that it expresses companion animal (e.g. dog) OX40 in a stable or transient manner.

The cell line may comprise a reporter gene and activation of OX40 by OX40L can be assessed using the reporter gene, e.g. by quantification of reporter gene expression. Suitable reporter genes are known to the skilled person and may include fluorescent markers or alkaline phosphatase (SEAP).

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, ABAB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non-limiting examples.

### EXAMPLES

### Example 1: Cloning Canine OX40L and OX40

OX40L Sequence Prediction

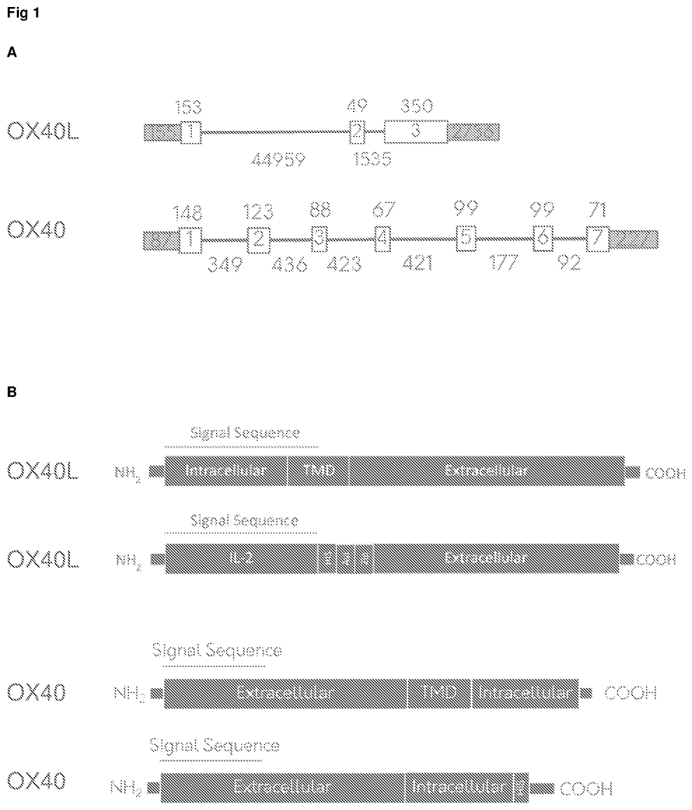

The nucleotide sequence for canine OX40L was predicted from the UCSC Genome Browser (University of Santa Cruz) Beagle reference genome based upon homology with human OX40L nucleotide sequences. Unlike many transmembrane proteins which consist of a 5′ terminus followed by the extracellular domain, OX40L structure is reversed (see FIG. 1A). This gave 3 predicted exons. FIG. 1B shows a schematic of a soluble form of OX40L (“OX40L_{EXT}”) formed by replacing the N terminal intracellular region with the IL-2 signal sequence to promote secretion from the cell. The corresponding nucleotide sequence is shown in FIG. 6B.

OX40 Sequence Prediction

The nucleotide sequence for canine OX40 was predicted from homology of the dog reference genome with human OX40. This gave 7 predicted exons with extracellular domain at the N terminus (FIG. 1A).

OX40L and OX40 Cloning

The sequence for both OX40L and OX40 were confirmed by cloning from PBMCs isolated from beagle blood using standard protocols. Briefly, Beagle whole blood was supplied by Envigo RMS (Alconbury, Huntingdon, UK) and PBMC's isolated using a Ficoll gradient. Briefly, 10 ml whole blood was diluted with 25 ml phosphate buffered saline (PBS) and layered onto 15 ml Ficoll Paque Plus (Sigma Aldrich) before centrifuging at 800 rcf for 10 min, room temp, with slow acceleration and no brake. The interphase disk was collected into PBS, the PBMC's counted, and then diluted at 10⁶ cells ml⁻1 in RPMI media (Sigma Aldrich)+1 μg ml⁻¹ IL-2.

To stimulate OX40 and OX40L expression, 5 mg ml⁻¹ Phytohaemagglutinin-L (PHA-L; Fisher Scientific) was added, and PBMC's harvested 1 and 4 days later. Total RNA was isolated from activated PBMC's with the QIAGEN RNeasy Mini Kit (Qiagen, Hilden, Del.) and standard procedures, yielding 160 μg-440 μg RNA following an on-column DNAse digestion. Specific cDNA amplification of OX40 and OX40L was undertaken using the SuperScript IV One-Step RT-PCR kit (ThermoFisher, Massachusetts, US) with 3′ and 5′ terminal primers generated from predicted OX40 and OX40L nucleotides sequences. Primer sequences are shown in table 2.

OX40 and OX40L PCR products were subcloned into pJET using the CloneJET PCR Cloning Kit (ThermoFisher), before being transferred to pcDNA3.1 for mammalian expression.

The nucleotide and amino acid sequence for Canine OX40L (sequence of cloned OX40L/Full length membrane form for cellular expression is shown in table 2 (SEQ ID NO:1 and 2).

For OX40, the presence of potential splice variants in the RT-PCR products was observed. To isolate the individual splice variant transcripts, the RT-PCR products were subcloned and two different transcripts were identified with sequences as shown in table 2 (nucleotide, amino acid translation), the short and the long variant (SEQ ID NO:3 to 6).

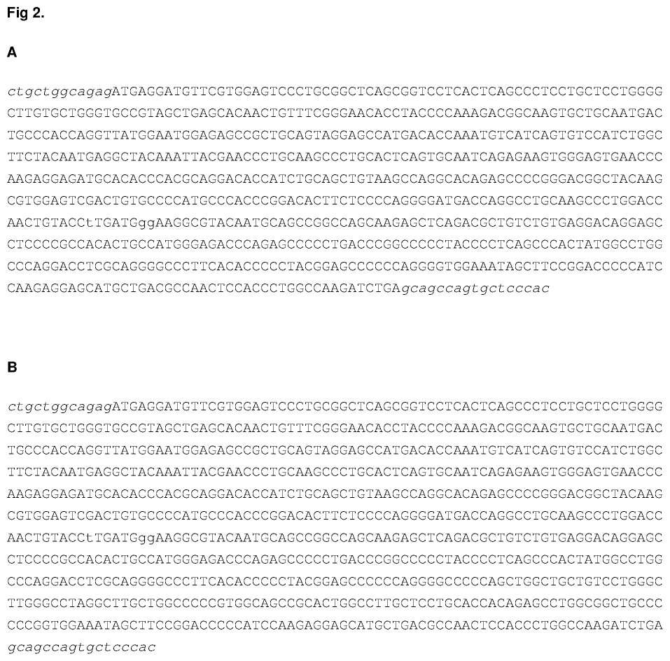

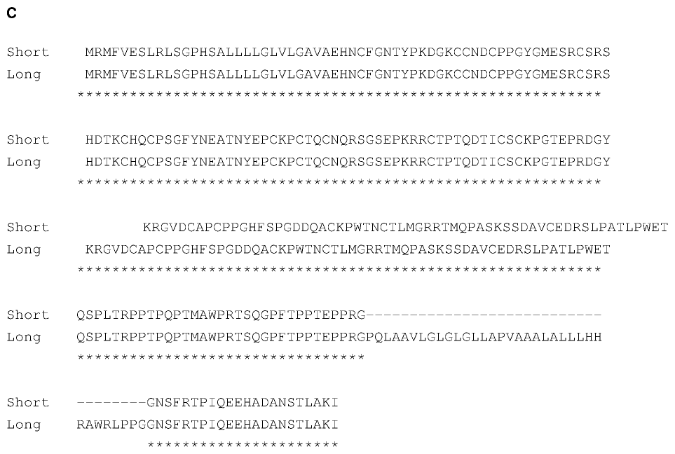

FIG. 2C shows an alignment of these splice variants. The long splice variant of OX40 consists of exons arranged in the sequence 1-2-3-4-5-6-7. The short splice variant does not contain exon 6, and has the arrangement 1-2-3-4-5-7

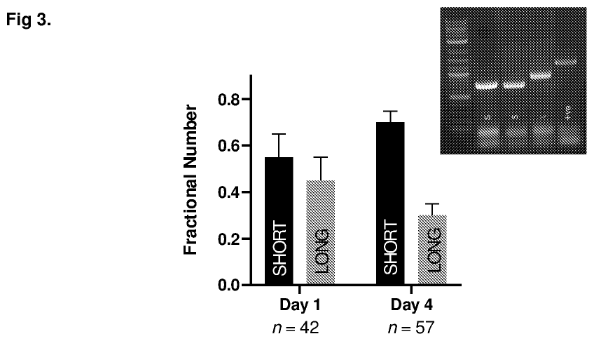

The relative abundance of OX40 splice-variants were assessed using a single-step PCR of pJET-OX40 colonies. Briefly, single colonies were picked from LB-amp plates into 10 μl OneTaq Quick-Load PCR mix (NEB) and amplified with the following PCR cycle: 1 cycle of 94° C. for 30 s; 30 cycles of 94° C. for 30 s, 61° C. for 30 s, 68° C. for 2 min; 1 cycle of 68° C. for 5 min. The abundance of the two splice variants was determined by assessing the relative lengths of the resultant PCR products (FIG. 3). The short splice variant codes for a truncated product. As this product lacks the transmembrane region in its entirety, it seems most likely that the resultant short variant would be secreted.

### Example 2: Expressing Canine OX40 and OX40L

Human embryonic kidney (HEK) 293 cells were grown on 90 mm round tissue culture plates as monolayers in DMEM/F12 (Life Technologies, California, US) supplemented with 10% fetal bovine serum (FBS; Sigma Aldrich) at 37° C. in a moist atmosphere containing 5% CO₂. HEK293 cells were transfected with plasmids encoding either OX40 (SEQ. ID 5) or OX40L (SEQ. ID 1) cDNA using polyethyleneimine (PEI MAX: 40 kDa, Polysciences Inc., Eppelheim, Germany). 30 μl of PEI MAX (1 mg ml⁻¹), 5 μg cDNA and 1 ml DMEM/F12 were incubated for 10 min at room temperature, added dropwise to a 90 mm plate of 70-80% confluent HEK293 cells, and incubated for 2-3 days. Stably transfected cells were selected 48 hrs later using a suitable antibiotic.

Mouse embryonic fibroblasts (MEF) were grown on 90 mm round tissue culture plates as monolayers in DMEM-high glucose (Life Technologies) supplemented with 10% FBS, 10% p-mercaptoethanol and 10% non-essential amino acids at 37° C. in a moist atmosphere and 5% CO₂. Cells were transfected with plasmids encoding OX40L cDNA (SEQ. ID 1) using Lipofectamine LTX (ThermoFisher Scientific, Waltham, Mass., USA) according to the manufacturer's recommended instructions. Stably transfected cells were selected 48 hrs later using a suitable antibiotic.

### Example 3: Immunisation—Using DNA and MEF

Ky9™ mice, substantially as described in WO2018/189520 and WO2020/074874, for example, were immunised. The transgenic mice have been modified compared to wild type mice by insertion of immunoglobulin heavy (IGH) chain and light chain (IGL) variable (V) region genes, IGH D region genes and IGH and IGL J region genes from a dog into a mouse allowing the production of antibody heavy chains that comprise a variable antibody region originating from the expression of canine DNA in the mouse, in combination with a constant region. The constant region may be the rodent immunoglobulin (IG) constant region, resulting in production of chimaeric heavy chains having canine variable region and a rodent constant region. Information concerning, or the nucleic acid comprising, the variable region of such chimaeric antibody chains may be used to generate fully canine antibodies, for therapeutic use in dogs for example. The rodent containing the canine DNA may also serve as an animal model for understanding of disease and testing of medicines.

For DNA immunisation, a prime and boost regime using hydrodynamic tail vein injection (HTVI) was performed and tissues harvested.

For cell-based immunisation, a prime and boost regime using MEF cells stably expressing OX40L was performed and tissues harvested.

A further immunisation regime was performed using HTVI DNA immunisation for the prime combined with OX40L-expressing MEF cell boosts.

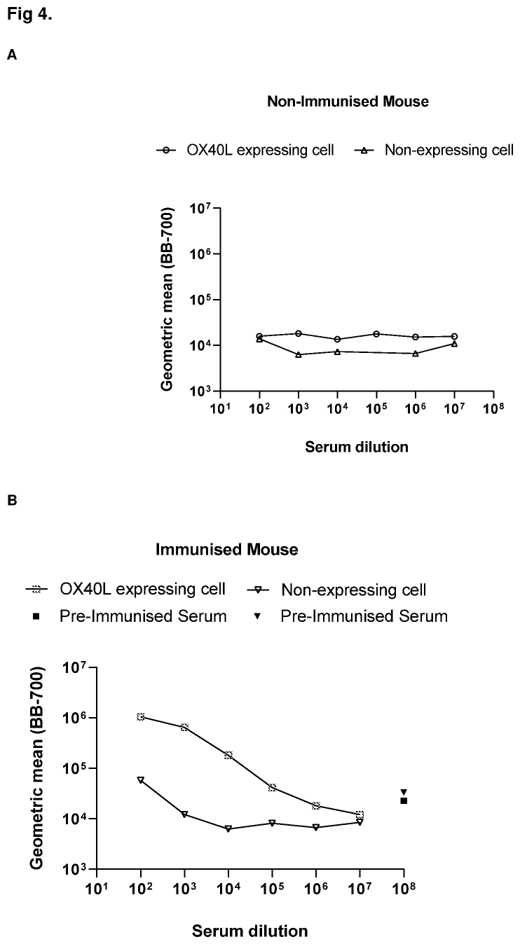

Determination of serum titres: Mice were bled prior to immunisation and 10 days after each subsequent boost. Serum and red blood cells were separated using microvette 200 Z-gel tubes (Starstedt AG & Co. KG, Germany) and titres of the OX40L-specific antibody response was evaluated using a BD Accuri C6 Flow Cytometer (Becton Dickinson, NJ, USA) or Beckman Coulter's CytoFLEX S. Post-immunisation serum was serially diluted in FACS buffer (PBS+3% FBS) and added to either 105 wild type cells or 105 of the same cells stably-expressing OX40L. Mouse antibodies were detected with 1/200 dilution of BB700 conjugated 2° monoclonal antibodies against isotypes IgG1, IgG2a, IgG2b (BD OptiBuild™, Becton Dickinson), and binding on these cells was compared to pre-immunisation serum (FIG. 4).

### Example 4: Isolation of Antibody Producing Cells

Tissue Isolation:

Spleens, lymph nodes and bone marrow were harvested from mice. Splenocytes were prepared by cutting the spleen into pieces and forcing these through a 45 μm cell strainer (Falcon) while rinsing with RPMI-1640 (Lonza, Basel, CH)+10% FBS on ice. A similar process was used for lymphocytes from lymph nodes. Bone marrow was collected from femur and tibia and flushing the marrow with RPMI-1640 using a 21-gauge needle, through a 45 μm cell strainer pre-wetted with RPMI-1640. All cell types were pelleted at 300 g for 5 min, before either being directly used for flow sorting or resuspended in FBS+10% dimethyl sulfoxide (DMSO) and being frozen at −150° C.

Cell Sorting:

Generally, antigen-specific splenic B cells can be captured by labelled antigen-VLPs or antigen protein probes because they express transmembrane antibodies on the cell surface. On the other hand, antigen-specific plasma cells are less easily labelled by antigen probes because of their dominant expression of secreted antibodies. Therefore, plasma cells isolated from the spleen or the bone marrow were proceeded to the next step of antibody sequence recovery without using antigen probes to isolate the antigen-specific subset of these populations. Cell surface co-expression of CD138 and CD267 (TACI) was used to identify the population of plasmablasts and plasma cells in bone marrow and spleen. Prior to antigen-specific cell sorting, B cell enrichment was undertaken using a mouse pan-B cell isolation kit according to the manufacturer's instructions (StemCell Technologies UK).

Antigen-specific cells can be captured by labelled VLPs that express target antigen upon their surface. VLPs are generated from HEK cells stably transfected with OX40L, and subsequently transiently transfected with the retrovirus gag protein, and fluorescently labelled MA (gag matrix fragment p15-GFP fusion protein); the gag expression enables VLP budding from cells, and MA labels the VLPs for fluorescence-detection. Surface antigens on VLPs are directly expressed from recombinant cells without any step of purification or modification, and are presented in a native form. Other mammalian cell lines, such as Chinese Hamster Ovary cells (CHO) or mouse embryonic fibroblasts (MEF) can also be used for VLP production.

Antigen-specific B cells can also be captured by labelled antigen protein probes. Monomeric OX40L protein containing OX40L extra cellular domain (ECD), or trimeric soluble OX40L extra cellular domain probes (shown in the schematic in FIG. 7 and with reference to Willett et al. Mol Immunol. 2009, 46(6); 1020-1030) were synthesised in expression vectors and expressed in CHO cells. Fc tagged probes were purified from culture supernatant using Mab select protein A resin (Cytiva) or by AKTA using Mab Select SuRe columns (Cytiva). HIS tagged trimeric OX40L protein was purified using HIS-Pur Ni-NTA resin (ThermoFisher).

Conjugation of OX40L ECD TNCc HIS (MW=62.143 kDa; trimeric) to Alexa Fluor 647 was carried out using the Microscale Protein Labeling Kit (Molecular Probes—Invitrogen catalogue number A30009) according to the manufacturer's protocol. Degree of labelling was determined using a NanoDrop spectrophotometer.

Similarly, the conjugation of monomeric dOX40L-mvhfc (MW=40.435 kDa) and OX40L ECD TNCc Fc (MW=280.980 kDa; hexameric) to Alexa Fluor 647 was carried out using the Alexa Fluor 647 Antibody labelling kit (Molecular Probes—Invitrogen catalog number A20186) following manufacturer's protocol for antibody labelling. Degree of labelling was determined using a NanoDrop spectrophotometer.

Different dilutions of both probes were tested on splenocytes of mice immunised with OX40L or with an unrelated immunogen in conjunction with OX40L overexpressing HEK 293 derived Gag-GFP virus-like particles (VLP) to identify and sort antigen specific B cells by flow cytometry. The optimal dilution displaying minimal background staining on un-relevant material was then used to for antigen-specific B cells identification and sorting by BD FACSAria Fusion cell sorter (BD Biosciences).

Markers including CD19, IgM, IgA, IgD and CD138 are then used to identify isotype-switched B cells enriched in cells that are responding to the immunisation.

Sorted cells are prepared for antibody profiling using the 10×Genomics Chromium Single Cell Immune Profiling system and the V(D)J Kit (10×Genomics) according to the manufacturer's instructions. Nucleotide sequences of expressed antibodies are determined by Illumina MiSeq sequencing with 600 cycles (2×300 cycles) or by Illumina iSeq, Miseq, MiniSeq, Nextseq, Hiseq 4000 or Novaseq sequencing with 2×150 cycles. The sequences are analysed using custom tools based on the pRESTO/Change-O (Yale University)/IgBlast (NCBI, USA) software to predict the germline sequence and hypermutation for each cell. The variable immunoglobulin region comprises a VDJ region of an immunoglobulin nucleotide sequence for heavy genes and a VJ region of an immunoglobulin nucleotide sequence for Igκ and Igλ. Within a clonal family there are subfamilies with shared mutations within their V(D)J segments that arise during immunoglobulin gene recombination and somatic hypermutation. Different clonal families that display unique V(D)J segment usage usually exhibit different binding characteristics. During recombination and hypermutation, cells whose antibodies have a higher affinity for an antigen are selected, and if low affinity clones from the same lineage have a neutralising function, the affinity usually increases with further mutations; for example, a clustered family is shown in FIG. 6 of WO2015/040401.

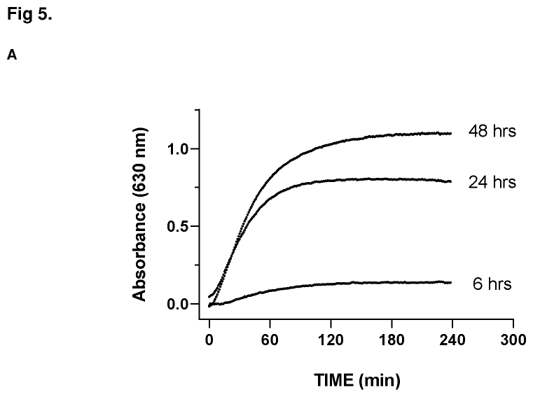

A clonal family is generally defined by related immunoglobulin heavy chain and/or light chain V(D)J sequences of two or more clonal cells. Related immunoglobulin heavy chain V(D)J sequences can be identified by their shared usage of V(D)J gene segments. An example of the analysis of antibody sequences of sorted Ag-specific single B-cells is shown in FIG. 5 of WO2015/040401, and shows antibody sequences that are arranged by heavy-chain V-gene family usage, and clustered to generate the displayed phylogenetic trees. From phylogenetic trees such as these, candidate clones are selected. The nucleic acid and amino acid sequences of candidate clones VH and VL and their corresponding CDRs are given in the Sequences table 2 below.

For instance, anti-canine OX40L mAbs PMX012, PMX014-018, PMX020-023 are all encoded by the same heavy chain V-gene (cIGHV3-5) and light chain V-gene (cIGLV3-3). Anti-canine OX40L mAb PMX013 is encoded by heavy chain V-gene cIGHV4-1 and light chain V-gene cIGLV3-3. PMX019 and PMX024 are encoded by heavy chain V-gene cIGHV3-2 and light chain V-gene cIGLV3-3.

PMX012 to PMX024 have the sequences set out in Table 2 below.

### Example 5: Generation of Monoclonal Antibodies from Single Cells

In some examples, the heavy chain and light chain V(D)J sequence of selected candidate clones are synthesised and cloned into expression vectors containing the genomic sequences of the human IgG4, dog IgG constant region, such as IgGB constant region, or the dog IGK or IGL constant regions, respectively. In some examples the IgG B constant region includes mutations that render the constant region effector deficient. Suitable sequences for these mutated Fc regions are given in the Sequence table below.

DNA encoding for canine heavy chain variable regions selected as described above are cloned in expression vectors upstream constant regions (CH1-hinge-CH2-CH3) of either human IgG4, or canine IgGB Def2 (SEQ ID NO:143) or Def7 (SEQ ID NO:148). DNA encoding for canine light chain variable regions are cloned in expression vectors upstream constant regions of either human kappa light chain, or canine kappa or lambda light chains.

The expression vectors encoding the heavy chain and light chain are co-transfected into a suitable mammalian cell line such as CHO cells to obtain stable expression. For antibody production, 6×10⁶ selected CHO cells are seeded in 3 ml culture media and incubated at 32° C., 5% CO₂ with shaking at 200 rpm. 4% HyClone Cell Boost 7a supplement+0.4% HyClone Cell Boost 7b supplement+1% glucose is added to the media on days 1, 4, 7 and 10. Culture supernatants are collected on day 12 and the IgG concentration determined on a protein A chip using surface plasmon resonance (Biacore 8K, Cytiva Life Sciences).

### Example 6: Binding Assays of OX40L Candidate Antibodies

Binding Assays: Binding of OX40L Candidate Antibodies to Cell Surface Expressed Canine OX40L Proteins.

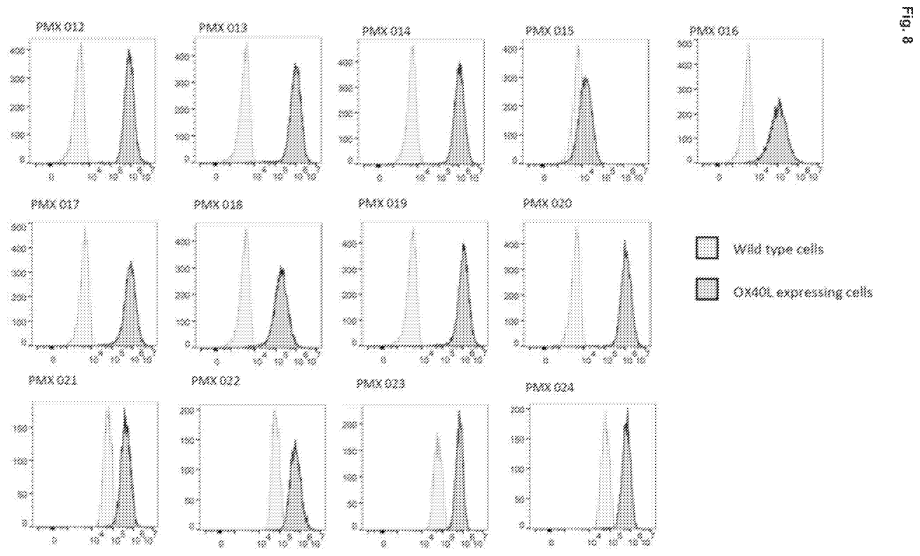

CHO cells supernatants containing candidate antibodies are diluted to 1, 5 or 10 μg/ml in FACS buffer (PBS containing 3% FBS) and screened for their ability to bind canine OX40L proteins expressed on cell surface. In brief, HEK293 or MEF cells (or any other commonly used cell lines) expressing canine OX40L at the cell surface are incubated with 100 μl of FACS buffer containing the candidate antibody for 30′ on ice. As a control the parental cell line (HEK293 or MEF cells not expressing OX40L) is also stained with the same antibody solution. After staining, cells are washed with 150 μl FACS buffer followed by centrifugation at 300 g for 3 min. Supernatant is removed, and the cell pellet is resuspended in FACS buffer containing a 1:1000 dilution of a fluorescently labelled secondary antibody recognising the human or canine Fc region of the test antibody for 30 min in the dark, followed by washing with 150 μl FACS buffer and centrifugation at 300 g for 3 mins. Cells are resuspended in FACS buffer and flow cytometry performed using either a Cytoflex (Beckton Dickinson) or an Accuri (Beckman Coulter) cytometer, followed by data analysis using FlowJo (FIG. 8).

Binding of Candidate Antibodies to Monomeric or Trimeric Antigen Probes.

OneComp eBeads (Invitrogen-ThermoFisher Cat #: 01-1111-41) are spherical particles that react with antibodies from different species and are immunoglobulin light chain-independent. The beads solution contains two populations of beads: a population capable of capturing any antibody and a control population that does not bind the antibody, resulting in a bimodal distribution. Therefore, the theoretical maximum binding is 50% as only half of the beads can bind the tested antibody.

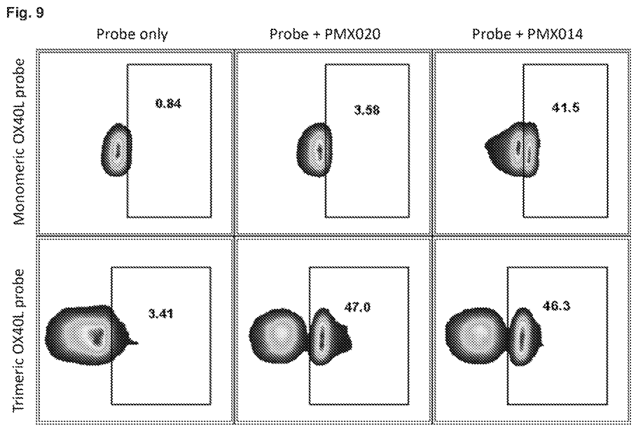

OneComp eBeads were incubated with OX40L antibodies. After one wash beads were subsequently incubated with the OX40L trimeric AlexaFluor 647-conjugated probe and washed again before acquisition on Beckman Coulter's CytoFLEX S. Data were analysed with FlowJo (FIG. 9). While PMX014 binds both probes, PMX020 only bind the trimeric probe, which recapitulates the natural conformation of OX40L expressed at cell surface. This experiment confirms that trimeric OX40L probes are better suited to use as both sorting and screening probes for the identification and screening of OX40L candidates.

Determination of Antibody Affinity by Binding on OX40L Expressed on Cell Surface

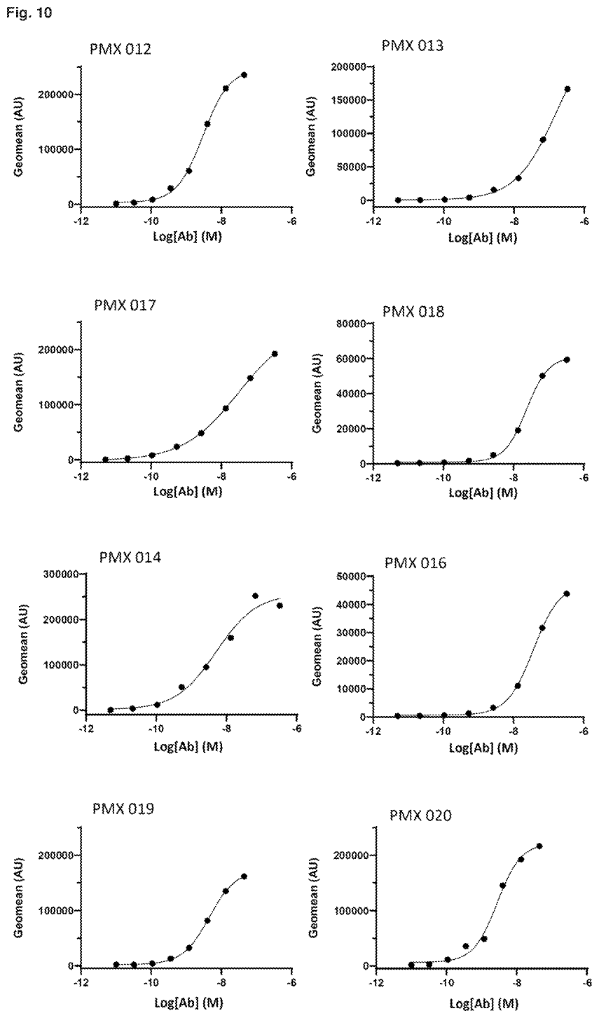

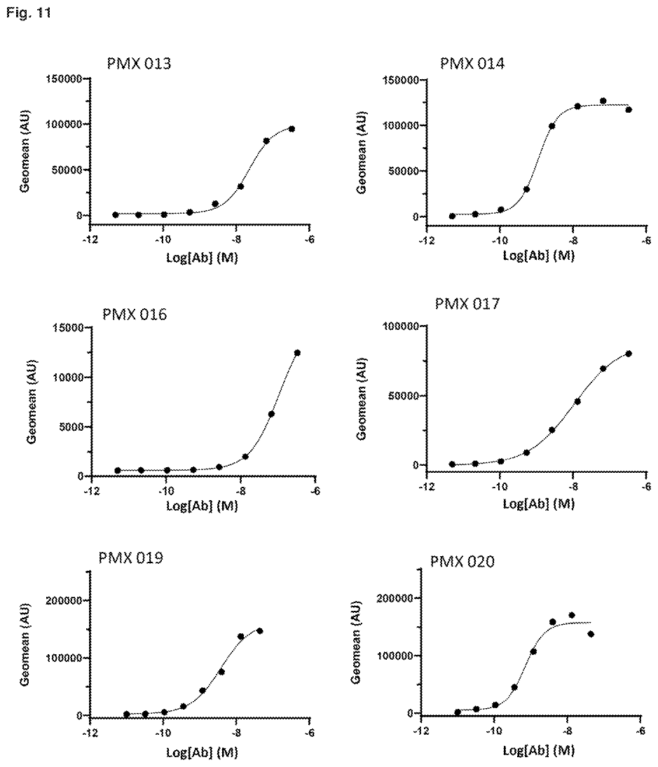

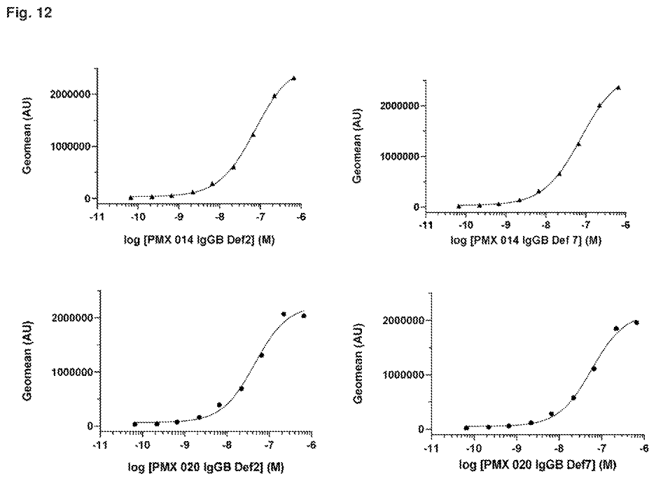

Specific binding to cells expressing OX40L is confirmed by purifying mAb from the CHO supernatant using protein A resin (MabSelect, Cytiva). In brief, MabSelect resin is washed in PBS and diluted to have a 10% slurry. An appropriate volume of the 10% slurry is added to the supernatant and incubated 5-10′ at RT before being loaded into a filter column. After 3 washes with PBS the antibodies are eluted using IgG elute buffer (Pierce) directly into neutralisation buffer (10 mM TRIS pH8.0), 8:1 IgG elute: neutralisation buffer ratio. Antibody solutions are quantified measuring absorbance at 280 nm, using a Nanodrop One (Thermo Fisher). Serial 1:3 dilutions of purified antibodies ranging from 6.6 μM to 66 pM are prepared in FACS buffer and are used to stain HEK293 or MEF cells (or any other commonly used cell lines) expressing canine OX40L. GeoMean values are plotted against antibody concentration and affinity (K_{d}) is calculated with GraphPad Prism using a four parameter logistic equation and variable slope (FIG. 10 to 12).

Determination of Antibody Affinity by Surface Plasmon Resonance (SPR)

Affinity (K_{d}) of OX40L antibodies is also measured by SPR, using a recombinant OX40L ECD-TNCc-HIS protein expressed in CHO cells (FIG. 7B).

OX40L ECD-TNCc-HIS protein is purified from CHO supernatant using HIS-Pur Ni NTA resin (ThermoFisher). In brief, CHO supernatant is buffer exchanged into PBS pH 8.0 containing 10 mM imidazole, using PD10 desalting columns. The buffer exchanged supernatant is then incubated 1 h at 4° C. with HIS-Pur Ni NTA resin in rotation. Resin is applied to a gravity filter column and washed 3 times with PBS pH 8.0 containing 10 mM imidazole before elution with 2.5 ml of PBS pH 8.0 containing 500 mM imidazole and buffer exchange to PBS pH7.4 using PD10 desalting columns.

To determine affinity by SPR (Biacore 8K, Cytivia), purified OX40L ECD-TNCc-HIS protein is covalently bound at low density to the surface of a CM5 sensor chip (Cytivia) by amine coupling, using the manufacturer's recommended protocol. Candidate antibodies are subsequently passed across the chip surface at a range of concentrations and affinity determined using the dedicated software (Biacore Insight Evaluation Software). Results are summarised in Table 3, SPR exp 1 and 2.

Alternatively, the extracellular domain of the OX40L protein (OX40L_{EXT}) is expressed in HEK293 cells and secreted into the extracellular media under the control of the IL-2 promoter (FIG. 1B). This is purified using a Ni⁺ column by utilising the HIS-tag. However, mammalian cells contain many His-containing proteins and often co-elute with the HIS-tagged protein of interest. To further purify the sample, the OX40L_{EXT}-containing eluate is later passed across a Streptavidin sensor chip (S Series SA Sensor Chip, GE Healthcare; Cat #BR100531) in the Biacore 8K, which captures the biotinylated Avi-tag of OX40L_{EXT} and allows remaining HIS-containing proteins to be washed away. Sensor chip loading continues until ˜500-1500 response units are seen. Amine coupling of OX40L_{EXT} to Biacore's Series S CM5 sensor chip (Cat #BR100530) can also be used, but as the reaction is non-specific it will result in immobilisation of non-target protein and could occlude potential OX40L target sites where immobilisation occurs at the epitope location.

CHO cell media containing secreted antibodies, or conditioned media (control), is later passed across the sensor and binding monitored. From these measurements the binding of Ab with the target protein can be assessed.

A subsequent injection of purified OX40 short splice variant (FIG. 1) enables the engagement or inhibition of OX40 and OX40L_{EXT}. Such a measurement can be used as an indication of whether Ab engagement prevents ligand-receptor interactions.

Alternatively, concentration and purification of the secreted antibodies can be performed by passing CHO cell media across the surface of a Series S protein-G sensor chip (Cat #29179315). Such an approach allows the sensor chip to be regenerated between experiments using conditions which do not alter the Ab, but may not allow the engagement of OX40L-OX40R to be subsequently assessed, as engagement with the Ab could induce a non-native orientation of OX40L and reduce binding through steric hindrance with the sensor chip.

### Example 7: Functional Assays for OX40 and OX40L Activity of Candidate Antibodies

HEK-Blue—NF_{K}B Functional Assay:

OX40 signals via the NFKB signalling pathway, enabling the use of human HEK-Blue™ Null1-v cells. HEK-Blue™ Null1-v cells express a secreted embryonic alkaline phosphatase (SEAP) reporter gene under the control of the IFN-3 minimal promoter fused to five NF-κB and AP-1 binding sites. Stimulation of HEK-Blue™ Null1-v cells with NF-κB and/or AP-1 activators induces the production of SEAP.

A HEK-Blue NF-kB reporter cell line is created by stable transfection of Null-1-v cells (hkb-null1v, InVivoGen) with canine OX40. A stable HEK293 cell line is created by transfection with OX40_{long} (HEK-OX40L). 1×10⁵ of HEK-Blue-OX40 cells were suspended in 2 ml DMEM+10% heat inactivated FBS and 100 μl transferred to each well of a 96-well plate. For agonist responses, 105 HEK-OX40L cells are similarly suspended in 2 ml DMEM+10% inactivated FBS and 100 μl added onto the HEK-Blue-OX40 cells in each well of the same 96 well plate. Activation of OX40 by OX40L is evaluated by the quantification of the induced secreted alkaline phosphatase (SEAP) reporter gene expression using a QuantiBlue detection kit (Invivogen, San Diego, USA) (FIG. 5).

Single-point inhibition of OX40-OX40L signalling is determined by adding specific anti-OX40L antibody or conditioned CHO-cell media to HEK-OX40L, 10 minutes prior to their addition to HEK-Blue-OX40. After incubation at 37° C. overnight, the maximal peak response is normalised to the response to OX40L alone. Concentration-dependence is determined for purified Ab by normalising responses to the maximum response in the absence of inhibitor and iteratively fitting the mean±SEM for a series of responses to the equation:

$${I}_{A} = {I}_{min} + \frac{{I}_{max} - {I}_{min}}{1 + \text{?}} \qquad ( Equ . 1 )$$

$$\text{?} \text{indicates text missing or illegible when filed}$$

where Aₘᵢₙ is the baseline response, Aₘₐₓ is the peak response evoked by agonist, A₅₀ is the concentration of A that yields a response equal to (Aₘₐₓ+Aₘᵢₙ)/2, x is the Ab concentration and n_{H} is the Hill slope.

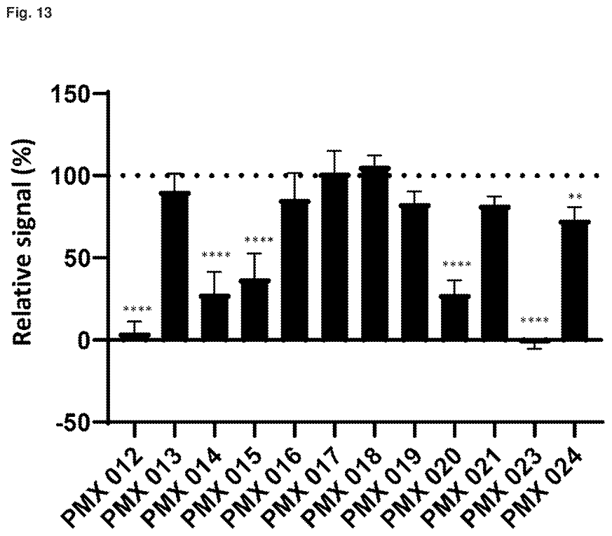

To determine the ability of OX40L selected candidates to reduce OX40 signalling 5000 HEK-blue-OX40 cells and 5000 HEK-OX40L cells are seeded in a 96 well plate in presence of protein A purified candidates at a fixed concentration of 6.6 μM (FIG. 13) or at a range of concentrations (2.2 μM-220 pM, FIG. 14). After 24 h incubation at 37° C., 20 μl of supernatant is collected and the presence of SEAP is assessed by the addition of 180 μl of QuantiBlue reagent and a further 4 hr incubation at 37° C., followed by measuring absorbance at 630 nm with a ClarioStar plate reader (BMG Labtech). Data is analysed using the dedicated MARS software version 3.40 R2 (BMG Labtech) and GraphPad Prism version 9.1. (FIGS. 13 and 14). A reduction in relative signal towards 0% in the presence of an antibody indicates reduced OX40 signalling. Corresponding IC50 values are shown in Table 4 below.

PBMC One-Way Mixed Lymphocyte Reaction (MLR):

PBMCs are isolated from two donors in the presence or absence of antibody. Stimulator cells are treated with mitomycin C (40 μg/ml for 25 min) to inhibit DNA synthesis. 50,000 stimulator cells are mixed 1:1 with responder cells in a 96 well plate with antibody dilutions, or medium only, at a final volume of 200 μl. Plates are incubated for 5-7 days at 37° C.

The response is often measured by ³[H]thymidine incorporation following a 0.5 μCi pulse. Cells are harvested 18 hours later and beta counted. Alternatively, a non-radioactive test can be used. The Cell Counting Kit-8 (CCK-8; Dojindo Molecular Technologies; Cat #CK04-11) allows sensitive colorimetric assays for the determination of cell viability in cell proliferation and cytotoxicity assays. Dojindo's water-soluble tetrazolium salt (WST-8) is reduced by dehydrogenase activities in cells to give a yellow-colour formazan dye, which is soluble in the tissue culture media. The amount of the formazan dye, generated by the activities of dehydrogenases in cells, is directly proportional to the number of living cells.

A CCK-8 solution is added to each well of the MLR, and incubated for 4 h before measuring the OD values in a microplate reader at 450 nm. The stimulation index is calculated as:

OD of responder cells in wells with stimulator cells added/OD of the same responders in wells containing responder cells only.

A further experimental protocol is shown below:

PBMCs are isolated from leukoreduction system chambers (NHSBT) using Ficoll-Paque plus (GE Healthcare) density gradient centrifugation. PBMC are pre-incubated with mitomycin C (Sigma) at 10 Mg/mL in PBS for one hour at 37° C. Cells are then washed 3 times in PBS centrifuging at 300×g for 3 minutes, aspirating the supernatant after each wash. Allogeneic PBMC (not treated with mitomycin C) are added to a 96-well plate in RPMI supplemented with 10% v/v FBS at a concentration of 2×10⁶/ml, 50 pL/well. Anti-OX40L antibodies are diluted in culture media and added to 96 well plate containing PBMC (not mitomycin C treated) at 50 pL/well. Mitomycin C treated PBMC are then added to allogeneic PBMC (not treated with mitomycin C) in 96-well plate at a final cell ratio in range of 1:1 to 4:1 mitomycin C treated to non mitomycin C based on number of cells/well. The cells are incubated for five days at 37° C./5% CO2. After five days TNF-o, IFN-γ, and IL-2 are measured by duoset ELISA (R&D Systems) according to manufacturer's recommendations. Proliferation is measured by CFSE dilution according to manufacturer's recommendations.

In Vitro PBMC Activation.

PBMCs are isolated from freshly drawn whole blood, with sodium heparin anticoagulant, using Ficoll-Paque plus (Cytiva, GE17-1440-02) density gradient centrifugation. In brief, canine blood is diluted 1:1 with phosphate buffer saline (PBS) and carefully layered on top of Ficoll-paque plus, centrifugated at 800 rcf for 20′ with slow acceleration and no break at the end. The top layer and interphase disk are diluted with PBS and centrifuged at 1300 rpm for 10′ to collect PBMC in the pellet which was washed a second time in PBS to remove all remnants of Ficoll. After a second centrifugation, PBMCs are resuspended in media (PBMC media=RPMI+10% heat inactivated foetal bovine serum+1% penicillin-streptomycin+1% non-essential amino acids+1% L-glutamine+1% sodium pyruvate+1% HEPES) supplemented with 50 ng/ml recombinant canine IL-2 (R&D systems) and incubated for 24 h at 37° C., 5% CO₂ before use or freezing.

Next day, PBMC are seeded in a 96 well plate in 200 μl/well of PBMC media supplemented with PHA (Sigma Aldrich) or ConA (ThermoFisher) and cultured with or without additional OX40L trimeric protein and/or OX40L antibodies at different concentrations at 37° C., 5% CO₂. 20 μl of supernatant is then collected at day 3-5 for cytokine quantification. IFN-γ is measured by ELISA (MABtech) according to manufacturer's recommendations.

Alternatively, PBMCs are isolated as described above and incubated for 24 h at 37° C., 5% CO₂ before use, and seeded next day in a 96 well plate precoated with a combination of anti-canine CD3 (Biorad) and CD28 (eBioscience) antibodies and cultured with or without additional OX40L trimeric protein and/or OX40L antibodies at different concentrations at 37° C., 5% CO₂. 20 μl of supernatant is then collected at day 3-5 for cytokines quantification. IFN-γ is measured by ELISA (MABtech) according to manufacturer's recommendations.

### Example 8 Fc Variants

Fc Variants Construction

For antibody production, DNA constructs were generated to encode chimeric antibodies comprising selected canine IgG constant regions fused to the variable regions of anti-human CD20 antibodies, Rituximab (see U.S. Pat. No. 5,736,137) or Ofatumumab (sequence available on Drugbank https://go.drugbank.com/drugs/DB06650).

The canine IgG-B mutant variants (Def 1 to 9, Seq ID 142 to 150) were generated by site directed mutagenesis. Specifically, human Ofatumumab or Rituximab variable region and canine IgG-B mutant variants are PCR amplified using Q5 high fidelity DNA polymerase and assembled into mammalian expression vector PetML5 using NEBuilder HIFI DNA Assembly (New England Biolabs). Ofa-VH-cIgGB WT Ofatumumab sequence is shown in SEQ ID NO. 151. In the expression vector, the heavy chain and the antibiotic resistant gene expression units are flanked by DNA transposon piggyBac terminal inverted repeats to mediate stable integration into host cells in the presence of piggyBac transposase. The expression vectors encoding the heavy chain and light chain are co-transfected into a suitable mammalian cell line such as CHO cells together with PiggyBac transposase to obtain stable expression. For antibody production, 3×10⁶ selected CHO cells are seeded in 3 ml culture media and incubated at 32° C., 5% CO₂ with shaking at 200 rpm. 4% HyClone Cell Boost 7a supplement+0.4% HyClone Cell Boost 7b supplement+1% glucose is added to the media on days 1, 4, 7 and 10. Culture supernatants are collected on day 12 and the IgG concentration determined using surface plasmon resonance using protein A chip (Biacore 8K, Cytiva Life Sciences).

Complement Dependent Cytotoxicity (CDC) Activity

Canine lymphoid tumour cell lines such as CLBL1 (University of Veterinary Medicine Vienna) or CLC (Umeki S. et al J. Vet. Med. Sci., 75:467-474 (2013) PubMed=23196801; DOI=10.1292/jvms.12-0448) may be used as target cells. These cells are stably transfected or nucleofected with an expression construct encoding for the human CD20 protein (Seq ID 152) to generate hCD20-expressing cells. The expression constructs were generated using the same method as described above for antibody expression.

Transfected cells were selected for puromycin resistance and hCD20^{high} (top 5%) cells were FACS sorted by staining for hCD20 expression using anti-human CD20 antibody (clone: 2H7, BioLegend).

To assess CDC activity, untransfected (wild type) target lymphoid cells or equivalent hCD20-expressing cells were used in a cell killing assay in which 5000 target cells per well of 96-well plate were incubated with anti-human CD20 canine Fc chimeric antibody as described above and canine complement preserved serum (BioIVT) at a final dilution of 1:12, for 2 hours at 37° C., 5% CO₂. The assay was set up using media (RPMI+1% L-glutamine+20% fetal bovine serum for CLBL-1 cells made using heat inactivated serum so that canine complement preserved serum would be the only source of complement.

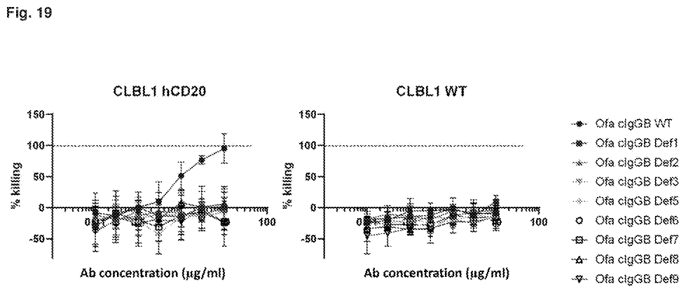

Live cells were then quantified using CellTitre-Glo® Luminescent Cell Viability Assay (Promega) following the assay protocol. This assay uses the ATP content of live cells as an indication of cell viability. Luminescence was measured on a CLARIOstar (BMG Labtech). Data was analyzed using MARS software (BMG Labtech) and the number of live cells remaining was used to calculate the percentage of killing in the presence of antibodies using Microsoft Excel. Background signal was obtained from a sample of cells treated with 1% triton (where no cells were left alive) and subtracted from the signal obtained from each test samples. Max signal (0% killing) was obtained from a sample of cells treated identically but where antibodies were omitted. Graphs were plotted in Microsoft Excel or GraphPad Prism. FIG. 19 shows the results of an exemplary CDC assay performed using hCD20 expressing CLBL1 cells and WT CLBL1 cells and canine IgGB WT or containing mutations Def1, 2, 3, 5, 6, 7, 8, 9. Antibodies were used at serial 1:3 dilutions ranging from 10 μg/ml to 0.01 10 μg/ml. All Def mutants tested (Def1, 2, 3, 5, 6, 7, 8, 9) completely abrogated the ability of canine IgGB WT to kill hCD20 CLBL1 cells by complement dependent cytotoxicity (FIG. 19).

TABLE 2
SEQUENCES All sequence for antibody and antibody fragments designated
as PMX and shown below are within the scope of the invention.

| SEQ | | |
|---|---|---|
| ID | | |
| NO: | Description | Sequence |
| 1 | Wild type canine | MEGVQPLDQNVGNTPGRRFQKNKVLLVAAIIQGLGLLLCFTYI |
| | OX40L amino acid | CLHFYASQVPPQYPPIQSIRVQFTRCENEKGCIITSPSKDETM |
| | | KVQDNSIIINCDGFYLISLKGYFSEELSLSLYYRKGRGPLFSLS |
| | | KVTSVDSIGVAYLAFKDKVYFNVTTHSTSYKDIQVNGGELILIH |
| | | QNPGGFCAY |
| 2 | Wild type canine | ATGGAAGGGGTCCAACCCCTGGACCAGAATGTGGGAAACA |
| | OX40L nucleic acid | CACCAGGGCGAAGATTCCAGAAGAACAAGGTATTGCTGGT |
| | | GGCAGCCATAATTCAGGGGCTGGGTCTGCTCCTGTGTTTC |
| | | ACCTACATCTGCCTGCACTTCTATGCTTCTCAGGTGCCGCC |
| | | TCAGTATCCTCCAATTCAAAGTATCAGAGTACAATTTACCA |
| | | GGTGTGAGAATGAGAAAGGTTGCATCATCACATCCCCAAG |
| | | CAAGGATGAAACTATGAAGGTGCAAGACAACTCAATCATCA |
| | | TTAACTGTGATGGGTTTTATCTCATCTCCCTGAAGGGTTAC |
| | | TTCTCTGAGGAGCTCAGCCTCAGCCTTTATTACCGAAAGG |
| | | GTCGGGGACCCCTCTTCTCTCTGAGCAAGGTCACATCTGT |
| | | TGACTCCATTGGAGTGGCCTATCTGGCTTTCAAGGACAAA |
| | | GTCTACTTTAATGTGACCACTCACAGTACCTCCTACAAAGA |
| | | CATCCAGGTGAATGGTGGGGAATTGATTCTCATTCATCAAA |
| | | ATCCTGGTGGCTTCTGTGCCTACTGA |
| 3 | Canine OX40 splice | ATGAGGATGTTCGTGGAGTCCCTGCGGCTCAGCGGTCCTC |
| | short variant nucleic | ACTCAGCCCTCCTGCTCCTGGGGCTTGTGCTGGGTGCCGT |
| | acid | AGCTGAGCACAACTGTTTCGGGAACACCTACCCCAAAGAC |
| | | GGCAAGTGCTGCAATGACTGCCCACCAGGTTATGGAATGG |
| | | AGAGCCGCTGCAGTAGGAGCCATGACACCAAATGTCATCA |
| | | GTGTCCATCTGGCTTCTACAATGAGGCTACAAATTACGAAC |
| | | CCTGCAAGCCCTGCACTCAGTGCAATCAGAGAAGTGGGAG |
| | | TGAACCCAAGAGGAGATGCACACCCACGCAGGACACCATC |
| | | TGCAGCTGTAAGCCAGGCACAGAGCCCCGGGACGGCTAC |
| | | AAGCGTGGAGTCGACTGTGCCCCATGCCCACCCGGACACT |
| | | TCTCCCCAGGGGATGACCAGGCCTGCAAGCCCTGGACCA |
| | | ACTGTACCtTGATGggAAGGCGTACAATGCAGCCGGCCAGC |
| | | AAGAGCTCAGACGCTGTCTGTGAGGACAGGAGCCTCCCC |
| | | GCCACACTGCCATGGGAGACCCAGAGCCCCCTGACCCGG |
| | | CCCCCTACCCCTCAGCCCACTATGGCCTGGCCCAGGACCT |
| | | CGCAGGGGCCCTTCACACCCCCTACGGAGCCCCCCAGGG |
| | | GTGGAAATAGCTTCCGGACCCCCATCCAAGAGGAGCATGC |
| | | TGACGCCAACTCCACCCTGGCCAAGATCTGA |
| 4 | Canine OX40 splice | MRMFVESLRLSGPHSALLLLGLVLGAVAEHNCFGNTYPKDGK |
| | short variant amino | CCNDCPPGYGMESRCSRSHDTKCHQCPSGFYNEATNYEPC |
| | acid | KPCTQCNQRSGSEPKRRCTPTQDTICSCKPGTEPRDGYKRG |
| | | VDCAPCPPGHFSPGDDQACKPWTNCTLMGRRTMQPASKSS |
| | | DAVCEDRSLPATLPWETQSPLTRPPTPQPTMAWPRTSQGPF |
| | | TPPTEPPRGGNSFRTPIQEEHADANSTLAKI |
| 5 | Canine OX40 splice | ATGAGGATGTTCGTGGAGTCCCTGCGGCTCAGCGGTCCTC |
| | long variant nucleic | ACTCAGCCCTCCTGCTCCTGGGGCTTGTGCTGGGTGCCGT |
| | acid | AGCTGAGCACAACTGTTTCGGGAACACCTACCCCAAAGAC |
| | | GGCAAGTGCTGCAATGACTGCCCACCAGGTTATGGAATGG |
| | | AGAGCCGCTGCAGTAGGAGCCATGACACCAAATGTCATCA |
| | | GTGTCCATCTGGCTTCTACAATGAGGCTACAAATTACGAAC |
| | | CCTGCAAGCCCTGCACTCAGTGCAATCAGAGAAGTGGGAG |
| | | TGAACCCAAGAGGAGATGCACACCCACGCAGGACACCATC |
| | | TGCAGCTGTAAGCCAGGCACAGAGCCCCGGGACGGCTAC |
| | | AAGCGTGGAGTCGACTGTGCCCCATGCCCACCCGGACACT |
| | | TCTCCCCAGGGGATGACCAGGCCTGCAAGCCCTGGACCA |
| | | ACTGTACCtTGATGggAAGGCGTACAATGCAGCCGGCCAGC |
| | | AAGAGCTCAGACGCTGTCTGTGAGGACAGGAGCCTCCCC |
| | | GCCACACTGCCATGGGAGACCCAGAGCCCCCTGACCCGG |
| | | CCCCCTACCCCTCAGCCCACTATGGCCTGGCCCAGGACCT |
| | | CGCAGGGGCCCTTCACACCCCCTACGGAGCCCCCCAGGG |
| | | GCCCCCAGCTGGCTGCTGTCCTGGGCTTGGGCCTAGGCT |
| | | TGCTGGCCCCCGTGGCAGCCGCACTGGCCTTGCTCCTGC |
| | | ACCACAGAGCCTGGCGGCTGCCCCCCGGTGGAAATAGCT |
| | | TCCGGACCCCCATCCAAGAGGAGCATGCTGACGCCAACTC |
| | | CACCCTGGCCAAGATCTGA |
| 6 | Canine OX40 splice | MRMFVESLRLSGPHSALLLLGLVLGAVAEHNCFGNTYPKDGK |
| | variant amino acid | CCNDCPPGYGMESRCSRSHDTKCHQCPSGFYNEATNYEPC |
| | | KPCTQCNQRSGSEPKRRCTPTQDTICSCKPGTEPRDGYKRG |
| | | VDCAPCPPGHFSPGDDQACKPWTNCTLMGRRTMQPASKSS |
| | | DAVCEDRSLPATLPWETQSPLTRPPTPQPTMAWPRTSQGPF |
| | | TPPTEPPRGPQLAAVLGLGLGLLAPVAAALALLLHHRAWRLP |
| | | PGGNSFRTPIQEEHADANSTLAKI |
| 7 | OX40 Forward primer | GGCAGAGATGAGGATGTTCG |
| 8 | OX40 Reverse primer | CACTGGCTGCTCAGATCTTGG |
| 9 | OX40L Forward | GCCACAGTTTTCATCTCCCT |
| 10 | OX40L Reverse | GCTTGGCTTAGGTGCAGC |
| 11 | PMX012 Heavy chain | GAGGTGCAGCTGGTGGAGTCTGGGGGAGACCTGGTGAAG |
| | variable region | CCTGGGGGGTCCCTGAGACTTGCCTGTGTGACCTCTGGAT |
| | nucleic acid sequence | TCACCTTCAGTAGCTATCACATGAACTGGGTCCGCCAGGC |
| | | TCCAGGGAAGGGGCTTCAGTGGGTCGCTTACATTAACACT |
| | | GGTGGAACTGTCACAACCTATGCAGACGCTATGAATGCAG |
| | | ACGCTGTGAGGGGCCGATTCACCATCTCCAGAGACAACGT |
| | | CAAGAACACGCTGTATCTTCAGATGAATAGACTGAGAGCC |
| | | GAGGACACGGCCGTATATTACTGTGCGCGCGGGTATGGG |
| | | GTCTTTGACTACTGGGGCCAGGGAACCCTGGTCACCGTCT |
| | | CCCCGG |
| 12 | PMX012 Heavy chain | EVQLVESGGDLVKPGGSLRLACVTSGFTFSSYHMNWVRQAP |
| | variable region amino | GKGLQWVAYINTGGTVTTYADAMNADAVRGRFTISRDNVKNT |
| | acid sequence | LYLQMNRLRAEDTAVYYCARGYGVFDYWGQGTLVTVSP |
| 13 | PMX012 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCCGTAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAACAGTG |
| | | ATAGGGCTAGTTGGGTATTCGGTGAAGGGACCCAGCTGAC |
| | | CGTCCTCG |
| 14 | PMX012 light chain | SYVLTQLPSKNVTLKQPAHITCGGDNIGSKSVHWYQQKLGQA |
| | variable region | PVLIIYYDSRRPTGIPERFSGANSGNTATLTISGALAEDEADYY |
| | amino acid sequence | CQVWDNSDRASWVFGEGTQLTVL |
| 15 | PMX012 heavy chain | GFTFSSY |
| | CDR1 | |
| 16 | PMX012 heavy chain | NTGGTV |
| | CDR2 | |
| 17 | PMX012 heavy chain | CARGYGVFDYW |
| | CDR3 | |
| 18 | PMX012 light chain | GGDNIGSKSVH |
| | CDR1 | |
| 19 | PMX012 light chain | YDSRRPT |
| | CDR2 | |
| 20 | PMX012 light chain | CQVWDNSDRASWVF |
| | CDR3 | |
| 21 | PMX013 Heavy chain | GAACTCACACTGCAGGAGTCAGGGCCAGGACTGGTGAAG |
| | variable region | CCCTCACAGACCCTCTCTCTCACCTGTGTTGTGTCCGGAG |
| | nucleic acid sequence | GCTCCGTCACCAGCAGTCACTACTGGAACTGGATCCGCCA |
| | | GCGCCCTGGGAGGGGACTGGAATGGATGGGGTCCTGGAC |
| | | AGGCGGAACAAACTACAACCCGGCATTCCAGGGACGCATC |
| | | TCTGTCACTTCTGACACGGCCAAGAACCAATTCTCCCTGCA |
| | | ACTGAGTTCCTTGACCACCGAGGACACGGCCGTGTATTAT |
| | | TGTGCACGAGGAGGCGGATATAGTGGCACCTGGAAGGATT |
| | | ACTATGTTATGGACTACTGGGGCCATGGCACCTCAGTCAT |
| | | CGTGTCCTCAG |
| 22 | PMX013 Heavy chain | ELTLQESGPGLVKPSQTLSLTCVVSGGSVTSSHYWNWIRQR |
| | variable region amino | PGRGLEWMGSWTGGTNYNPAFQGRISVTSDTAKNQFSLQLS |
| | acid sequence | SLTTEDTAVYYCARGGGYSGTWKDYYVMDYWGHGTSVIVSS |
| 23 | PMX013 light chain | TCCTATGTGCTGACACAGCTGCCATCCAAAAATGTGACCCT |
| | variable region | GAACCAGCCGGCCCACATCACCTGTGGGGGAGACAACCTT |
| | nucleotide sequence | GGAAGTAAAAGTGTTCACTGGTACCAGCAGAAGCTGGGCC |
| | | AGGCCCCTGTACTGATTATCTATTTTGATACCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 24 | PMX013 light chain | SYVLTQLPSKNVTLNQPAHITCGGDNLGSKSVHWYQQKLGQ |
| | variable region | APVLIIYFDTSRPTGIPERFSGANSGNTATLTISGALAEDEADY |
| | amino acid sequence | YCQVWDSSAKASVFGGGTHLTVL |
| 25 | PMX013 heavy chain | GGSVTSSH |
| | CDR1 | |
| 26 | PMX013 heavy chain | TGG |
| | CDR2 | |
| 27 | PMX013 heavy chain | CARGGGYSGTWKDYYVMDYW |
| | CDR3 | |
| 28 | PMX013 light chain | GGDNLGSKSVH |
| | CDR1 | |
| 29 | PMX013 light chain | FDTSRPT |
| | CDR2 | |
| 30 | PMX013 light chain | CQVWDSSAKASVF |
| | CDR3 | |
| 31 | PMX014 Heavy chain | GAGGTGCAGCTGGTGGAGTCTGGGGGAGACCTGGTGAAG |
| | variable region | CCTGGGGGGTCCCTGAGACTTTCCTGTGTGGCCTCTGGAT |
| | nucleic acid sequence | TCACATTCAGTAACTTCCACATGAGTTGGGTCCGCCAGGCT |
| | | CCAGGGAAGGGGCTTCAGTGGGTCGCATACATTAACAGTG |
| | | GTGGATTTAACATAAATTATGAAGACGCTGTGAGGGGCCG |
| | | CTTCACCATCTCCAGAGACAACGCCAAGAACACGTTGTATC |
| | | TTCAGATGAACAGCCTGAGAGCCGAAGACACGGCCATTTA |
| | | TTACTGTGCGCGTGATTGGGATACACATTTGGATACGAACT |
| | | GGTTCTACTACTGGGGCCAAGGGACCCTGGTCACTGTGTC |
| | | CTCAG |
| 32 | PMX014 Heavy chain | EVOLVESGGDLVKPGGSLRLSCVASGFTFSNFHMSWVRQAP |
| | variable region amino | GKGLQWVAYINSGGFNINYEDAVRGRFTISRDNAKNTLYLQM |
| | acid sequence | NSLRAEDTAIYYCARDWDTHLDTNWFYYWGQGTLVTVSS |
| 33 | PMX014 light chain | TCCTATGTGCTGACACAGCTGCCATCCAAAAATGTGACCCT |
| | variable region | GAAGCAGTCGGCCCACATCACCTGTCGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACATCAGTG |
| | | CTAAGGCTAGTGTGTTCGGCGGAGGCACCCATCTGACCGT |
| | | CCTCG |
| 34 | PMX014 light chain | SYVLTQLPSKNVTLKQSAHITCRGDNIGSKSVHWYQQKLGQA |
| | variable region | PVLIIYYDSSRPTGIPERFSGANSGNTATLTISGALAEDEADYY |
| | amino acid sequence | CQVWDISAKASVFGGGTHLTVL |
| 35 | PMX014 heavy chain | GFTFSNF |
| | CDR1 | |
| 36 | PMX014 heavy chain | NSGGFN |
| | CDR2 | |
| 37 | PMX014 heavy chain | CARDWDTHLDTNWFYYW |
| | CDR3 | |
| 38 | PMX014 light chain | RGDNIGSKSVH |
| | CDR1 | |
| 39 | PMX014 light chain | YDSSRPT |
| | CDR2 | |
| 40 | PMX014 light chain | CQVWDISAKASVF |
| | CDR3 | |
| 41 | PMX015 Heavy chain | GAGGTGCAGCTGGTGGAGTCTGGGGGAGACCTGGTGAAG |
| | variable region | CCTGGGGGGTCCCTGAGAATTTCCTGTGTGGCCTCTGGAT |
| | nucleic acid sequence | TCACCTTCAGTTACTACCACATGAGCTGGGTCCGCCAGGC |
| | | TCCAGGGAAGGGGCTTCAGTGGGTCGCATATATCAACAGT |
| | | GGTGGATTTAGTACAAACTATGCAGACGCTGTGAAGGGCC |
| | | GATGCTCCATCTCCAGAGACAATGGCAAGAACACGCTGTA |
| | | TCTTCAGATGAACAGCCTGAGACCCGAGGACACGGGCGTT |
| | | TATTATTGTGCGAGTGAAAGTCGTTGGGGGGATTCTTACAG |
| | | TGGTATGACCTACTGGGGCCATGGCACTTCACTCTTCGTG |
| | | TCCTCAG |
| 42 | PMX015 Heavy chain | EVQLVESGGDLVKPGGSLRISCVASGFTFSYYHMSWVRQAP |
| | variable region amino | GKGLQWVAYINSGGFSTNYADAVKGRCSISRDNGKNTLYLQ |
| | acid sequence | MNSLRPEDTGVYYCASESRWGDSYSGMTYWGHGTSLFVSS |
| 43 | PMX015 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTTCTGATTATCTATTATGATAACAGCAGGCCG |
| | | ACAGGGATCCCTGAGCGATTCTCCGGCGCCAAGTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 44 | PMX015 light chain | SYVLTQLPSKNVTLKQPAHITCGGDNIGSKSVHWYQQKLGQA |
| | variable region | PVLIIYYDNSRPTGIPERFSGAKSGNTATLTISGALAEDEADYY |
| | amino acid sequence | CQVWDSSAKASVFGGGTHLTVL |
| 45 | PMX015 heavy chain | GFTFSYY |
| | CDR1 | |
| 46 | PMX015 heavy chain | NSGGFS |
| | CDR2 | |
| 47 | PMX015 heavy chain | CASESRWGDSYSGMTYW |
| | CDR3 | |
| 48 | PMX015 light chain | GGDNIGSKSVH |
| | CDR1 | |
| 49 | PMX015 light chain | YDNSRPT |
| | CDR2 | |
| 50 | PMX015 light chain | CQVWDSSAKASVF |
| | CDR3 | |
| 51 | PMX016 Heavy chain | GAGGTGCAGCTGGTGGAGTCTGGGGGAGACCTGGTGAAG |
| | variable region | CCGGGGGGGTCCCTGAGACTTTCCTGTGTGGCCTCTGGAT |
| | nucleic acid sequence | TCACCTTCAGTAATTACCACATGAACTGGGTCCGCCAGGCT |
| | | CCAGGGAAGGGGCTTCAGTGGGTCGCATACATTACCAGTG |
| | | ATGGAATTGTTTCAAGCTACGCAGACGTTGTGAAGGGCCG |
| | | ATTCACCATCTCCAGAGACAACGCCAAGAACACGCTTTTTC |
| | | TTCAGATGAACAGCCTGAGAGCCGAGGACACGGCCGTGTA |
| | | TTATTGTGCGAGTGGGTTGTTTTTAGTAGTTGGGGGGGGG |
| | | ACCTTCTGGGGCCAGGGAACCCTGGTCACCGTCTCCTCAG |
| 52 | PMX016 Heavy chain | EVQLVESGGDLVKPGGSLRLSCVASGFTFSNYHMNWVRQAP |
| | variable region amino | GKGLQWVAYITSDGIVSSYADVVKGRFTISRDNAKNTLFLQM |
| | acid sequence | NSLRAEDTAVYYCASGLFLVVGGGTFWGQGTLVTVSS |
| 53 | PMX016 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTCTGATAGTAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 54 | PMX016 light chain | SYVLTQLPSKNVTLKQPAHITCGGDNIGSKSVHWYQQKLGQA |
| | variable region | PVLIIYSDSSRPTGIPERFSGANSGNTATLTISGALAEDEADYY |
| | amino acid sequence | CQVWDSSAKASVFGGGTHLTVL |
| 55 | PMX016 heavy chain | GFTFSNY |
| | CDR1 | |
| 56 | PMX016 heavy chain | TSDGIV |
| | CDR2 | |
| 57 | PMX016 heavy chain | CASGLFLVVGGGTFW |
| | CDR3 | |
| 58 | PMX016 light chain | GGDNIGSKSVH |
| | CDR1 | |
| 59 | PMX016 light chain | SDSSRPT |
| | CDR2 | |
| 60 | PMX016 light chain | CQVWDSSAKASVF |
| | CDR3 | |
| 61 | PMX017 Heavy chain | GAGGTGCAACTGGTGGAGTCTGGGGGAGACCTTGTGAAG |
| | variable region | CCTGGGGGGTCCCTGAGACTTTCCTGTGTGGCCTCTGGAT |
| | nucleic acid sequence | TCACCTTCAGTAGTTACCACATGAGCTGGGTCCGCCAGGC |
| | | TCCAGGGAAGGGGCTTCAGTGGGTCGCATACATTGCCAGT |
| | | GGTGGTACTGTCACAACCTATGCAGACGCTGTGAGGGGCC |
| | | GATTCACCATCTCCAGAGACAACGCCAAGAATATGTTGTAT |
| | | CTTCAGATGAACAGCCTGAGAGCCGAGGACTCGGCCGTAT |
| | | ATTACTGTACGAGGTGGAAGGGTGGGACTTTTGGCTATGG |
| | | TATGGACTACTGGGGCCATGGCACCTCACTCTTCGTGTCC |
| | | TCAG |
| 62 | PMX017 Heavy chain | EVQLVESGGDLVKPGGSLRLSCVASGFTFSSYHMSWVRQAP |
| | variable region amino | GKGLQWVAYIASGGTVTTYADAVRGRFTISRDNAKNMLYLQM |
| | acid sequence | NSLRAEDSAVYYCTRWKGGTFGYGMDYWGHGTSLFVSS |
| 63 | PMX017 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAACAACAGGCCG |
| | | GCAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 64 | PMX017 light chain | SYVLTQLPSKNVTLKQPAHITCGGDNIGSKSVHWYQQKLGQA |
| | variable region | PVLIIYYDNNRPAGIPERFSGANSGNTATLTISGALAEDEADYY |
| | amino acid sequence | CQVWDSSAKASVFGGGTHLTVL |
| 65 | PMX017 heavy chain | GFTFSSY |
| | CDR1 | |
| 66 | PMX017 heavy chain | ASGGTV |
| | CDR2 | |
| 67 | PMX017 heavy chain | CTRWKGGTFGYGMDYW |
| | CDR3 | |
| 68 | PMX017 light chain | GGDNIGSKSVH |
| | CDR1 | |
| 69 | PMX017 light chain | YDNNRPA |
| | CDR2 | |
| 70 | PMX017 light chain | CQVWDSSAKASVF |
| | CDR3 | |
| 71 | PMX018 Heavy chain | GAGGTGCAGCTGGTGGAGTCTGGGGGAGACCTGGTGAAG |
| | variable region | CCTGGGGGGTCCCTGAGACTTTCCTGTGTGGCCTCTGGAT |
| | nucleic acid sequence | TCACCTTCAGTAACTACCACATGAACTGGGTCCGCCAGGC |
| | | TCCAGGGAAGGGGCTTCAGTGGGTCGCATACATTACCAAT |
| | | GATGGAATTGTTTCAAGCTACGCAGACGCTGTGAAGGGCC |
| | | GATTCACCATCTCCAGAGACAACGCCAAGAACACGCTTTAT |
| | | CTTCAGATGAACAGCCTGAGAGTCGAGGACACGGCCGTGT |
| | | ATTACTGTGCGAGTGGATTGTTTCTAGTAGTTGGGGGGGG |
| | | GACCTTCTGGGGCCAGGGAACCCTGGTCACCGTCTCCTCA |
| | | G |
| 72 | PMX018 Heavy chain | EVQLVESGGDLVKPGGSLRLSCVASGFTFSNYHMNWVRQAP |
| | variable region amino | GKGLQWVAYITNDGIVSSYADAVKGRFTISRDNAKNTLYLQM |
| | acid sequence | NSLRVEDTAVYYCASGLFLVVGGGTFWGQGTLVTVSS |
| 73 | PMX018 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCATAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 74 | PMX018 light chain | SYVLTQLPSKNVTLKQPAHITCGGDNIGSKSVHWYQHKLGQA |
| | variable region | PVLIIYYDSSRPTGIPERFSGANSGNTATLTISGALAEDEADYY |
| | amino acid sequence | CQVWDSSAKASVFGGGTHLTVL |
| 75 | PMX018 heavy chain | GFTFSNY |
| | CDR1 | |
| 76 | PMX018 heavy chain | TNDGIV |
| | CDR2 | |
| 77 | PMX018 heavy chain | CASGLFLVVGGGTFW |
| | CDR3 | |
| 78 | PMX018 light chain | GGDNIGSKSVH |
| | CDR1 | |
| 79 | PMX018 light chain | YDSSRPT |
| | CDR2 | |
| 80 | PMX018 light chain | CQVWDSSAKASVF |
| | CDR3 | |
| 81 | PMX019 Heavy chain | GAGGTACAACTGGTGGAGTCGGGGGGAGACCTGGTGAAG |
| | variable region | CCTGGGGGGTCCCTGAGACTCTCCTGTGTGGCCTCTGGAT |
| | nucleic acid sequence | TCACCTTCAGTAGCAACTACATGAGCTGGATCCGCCAGGC |
| | | TCCAGGGAAGGGGCTGCAGTGGGTCTCACAAATTAGCGGT |
| | | GATGGAATTTACACAAACTACGCAGACGCTATGAAGGGCC |
| | | GATTCACCATCTCCAGAGACAATGCCAAGAACACGCTGTAT |
| | | CTGCAGATGAACAGCCTGAGAGATGAGGACACGGCACTAT |
| | | ATTACTGTGCAACTGGGATATACCCCAATGCTTTTGGTTAC |
| | | TGGGGCCAGGGCACCCTGGTCACTGTCTCCTCAG |
| 82 | PMX019 Heavy chain | EVQLVESGGDLVKPGGSLRLSCVASGFTFSSNYMSWIRQAP |
| | variable region amino | GKGLQWVSQISGDGIYTNYADAMKGRFTISRDNAKNTLYLQM |
| | acid sequence | NSLRDEDTALYYCATGIYPNAFGYWGQGTLVTVSS |
| 83 | PMX19 light chain | TCCTATGTGCTGACACAGTTGTCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGC |
| | | GCTAATGTGTTCGGCGGAGGCACCCATCTGACCGTCCTCG |
| 84 | PMX019 light chain | SYVLTQLSSKNVTLKQPAHITCGGDNIGSKSVHWYQQKLGQA |
| | variable region | PVLIIYYDSSRPTGIPERFSGANSGNTATLTISGALAEDEADYY |
| | amino acid sequence | CQVWDSSANVFGGGTHLTVL |
| 85 | PMX019 heavy chain | GFTFSSN |
| | CDR1 | |
| 86 | PMX019 heavy chain | SGDGIY |
| | CDR2 | |
| 87 | PMX019 heavy chain | CATGIYPNAFGYW |
| | CDR3 | |
| 88 | PMX019 light chain | GGDNIGSKSVH |
| | CDR1 | |
| 89 | PMX019 light chain | YDSSRPT |
| | CDR2 | |
| 90 | PMX019 light chain | CQVWDSSANVF |
| | CDR3 | |
| 91 | PMX020 Heavy chain | GAGGTGCAGCTGGTGGAGTCTGGGGGAGACCTGGTGAAG |
| | variable region | CCTGGGGGGTCCCTGAGAATTTCCTGTGTGGCTTCTGGAT |
| | nucleic acid sequence | TCACCTTCAGTAGCTACCACATGAACTGGGTCCGCCAGGC |
| | | TCCAGGGAAGGGGCTTCAGTGGGTCGCACACATTAGCAGT |
| | | GGTGGAACTTTCACAAGTTATGCAGACGCTGTGAAGGGCC |
| | | GATTCACCATCTCCAGAGACAACGCCAAGAACACGCTCTAT |
| | | CTTCAGATGATCAGCCTGAGAGCCGAGGACACGGCCGTGT |
| | | ATTACTGTGCGAGTGGGTTGTTTCTAGTAGTTGGGGGGGG |
| | | GAACTACTGGGGCCGGGGAACCCTGGTCACCGTCTCCTCA |
| | | G |
| 92 | PMX020 Heavy chain | EVQLVESGGDLVKPGGSLRISCVASGFTFSSYHMNWVRQAP |
| | variable region amino | GKGLQWVAHISSGGTFTSYADAVKGRFTISRDNAKNTLYLQM |
| | acid sequence | ISLRAEDTAVYYCASGLFLVVGGGNYWGRGTLVTVSS |
| 93 | PMX020 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTCTGATAGCAGCAGGCC |
| | | GACAGGGATCCCTGAGCGATTCTCCGGCGCCAACTCGGG |
| | | GAACACGGCCACCCTGACCATCAGCGGGGCCCTGGCCGA |
| | | GGACGAGGCTGACTATTACTGCCAGGTGTGGGACAGCAGT |
| | | GCTAGTGTGTTCGGCGGAGGCACCCATCTGACCGTCCTCG |
| 94 | PMX020 light chain | SYVLTQLPSKNVTLKQPAHITCGGDNIGSKSVHWYQQKLGQA |
| | variable region | PVLIIYSDSSRPTGIPERFSGANSGNTATLTISGALAEDEADYY |
| | amino acid sequence | CQVWDSSASVFGGGTHLTVL |
| 95 | PMX020 heavy chain | GFTFSSY |
| | CDR1 | |
| 96 | PMX020 heavy chain | SSGGTF |
| | CDR2 | |
| 97 | PMX020 heavy chain | CASGLFLVVGGGNYW |
| | CDR3 | |
| 98 | PMX020 light chain | GGDNIGSKSVH |
| | CDR1 | |
| 99 | PMX020 light chain | SDSSRPT |
| | CDR2 | |
| 100 | PMX020 light chain | CQVWDSSASVF |
| | CDR3 | |
| 101 | PMX021 Heavy chain | GAGGTGCAACTGGTGGAGTCTGGGGGAGACCTGGTGAAG |
| | variable region | CCTGGGGGGTCCCTGAGACTTTCCTGTGTGGCCTCTGGAT |
| | nucleica cid sequence | TCACCTTCAGTAACTACCACATGAGCTGGGTCCGCCAGGC |
| | | TCCAGGGAAGGGGCTTCAGTGGGTCGCATACATTAACAGT |
| | | GATGGGAGAGTCACCACCTATTCAGACGCTGTGAAGGGCC |
| | | GATTCACCATCTCCAGAGACAACGCCAAGAACACGCTGTG |
| | | TCTTCAGATGAACAGCCTGAGAGCCGAGGACACGGCCGTG |
| | | TATTACTGTGCGAGGTGGAGGGGTGGGACTTTTGGCTATG |
| | | GTATGGACTACTGGGGCCATGGCACCTCACTCTTCGTGTC |
| | | TTCAG |
| 102 | PMX021 Heavy chain | EVQLVESGGDLVKPGGSLRLSCVASGFTFSNYHMSWVRQAP |
| | variable region amino | GKGLQWVAYINSDGRVTTYSDAVKGRFTISRDNAKNTLCLQM |
| | acid sequence | NSLRAEDTAVYYCARWRGGTFGYGMDYWGHGTSLFVSS |
| 103 | PMX021 light chain | TCCTATGTGCTGACACAGCTGCCATCCAAAAATGTGACCCT |
| | variable region | GAAGCAGCCGGCCCACATCACCTGTGGGGGAGACAATATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 104 | PMX021 light chain | SYVLTQLPSKNVTLKQPAHITCGGDNIGSKSVHWYQQKLGQA |
| | variable region | PVLIIYYDSSRPTGIPERFSGANSGNTATLTISGALAEDEADYY |
| | amino acid sequence | CQVWDSSAKASVFGGGTHLTVL |
| 105 | PMX021 heavy chain | GFTFSNY |
| | CDR1 | |
| 106 | PMX021 heavy chain | NSDGRV |
| | CDR2 | |
| 107 | PMX021 heavy chain | CARWRGGTFGYGMDYW |
| | CDR3 | |
| 108 | PMX021 light chain | GGDNIGSKSVH |
| | CDR1 | |
| 109 | PMX021 light chain | YDSSRPT |
| | CDR2 | |
| 110 | PMX021 light chain | CQVWDSSAKASVF |
| | CDR3 | |
| 111 | PMX022 Heavy chain | GAGGTGCAGCTGGTGGAGTCTGGGGGAGACCTGGTGAAG |
| | variable region | CCTGGGGGGTCCCTGAGACTTTCCTGTGTGGCCTCTGGAT |
| | nucleic acid sequence | TCACCTTCAGTAACTACCACATGAGCTGGGTCCGCCAGGC |
| | | TCCAGGGAAGGGGCTTCAGTGGGTCGCATACATTAATAGT |
| | | GATGGAAGAATTACAACCTATGCAGACGCTGTGAAGGGCC |
| | | GATTCACCATCTCCAGAGACAACGCCAAGAACACGCTGTA |
| | | TCTTCAGATGAACAGCCTGAGAGTCGAGGACACGGCCGTG |
| | | TATTACTGTGCGAGGTGGAGGGGTGGGACTTTTGGCTATG |
| | | GTATGGACTACTGGGGCCATGGCACCTCACTCTTCGTGTC |
| | | CTCAG |
| 112 | PMX022 Heavy chain | EVQLVESGGDLVKPGGSLRLSCVASGFTFSNYHMSWVRQAP |
| | variable region amino | GKGLQWVAYINSDGRITTYADAVKGRFTISRDNAKNTLYLQM |
| | acid sequence | NSLRVEDTAVYYCARWRGGTFGYGMDYWGHGTSLFVSS |
| 113 | PMX022 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAAGGCTAGTGTGTTCGGCGGAGGCACCCATCTGACCGT |
| | | CCTCG |
| 114 | PMX022 light chain | SYVLTQLPSKNVTLKQPAHITCGGDNIGSKSVHWYQQKLGQA |
| | variable region | PVLIIYYDSSRPTGIPERFSGANSGNTATLTISGALAEDEADYY |
| | amino acid sequence | CQVWDSSAKASVFGGGTHLTVL |
| 115 | PMX022 heavy chain | GFTFSNY |
| | CDR1 | |
| 116 | PMX022 heavy chain | NSDGRI |
| | CDR2 | |
| 117 | PMX022 heavy chain | CARWRGGTFGYGMDYW |
| | CDR3 | |
| 118 | PMX022 light chain | GGDNIGSKSVH |
| | CDR1 | |
| 119 | PMX022 light chain | YDSSRPT |
| | CDR2 | |
| 120 | PMX022 light chain | CQVWDSSAKASVF |
| | CDR3 | |
| 121 | PMX023 Heavy chain | GAGGTGCAGCTGGTGGAGTCTGGGGGAGACCTGATGAAG |
| | variable region | CCTGGGGGGTCCCTGAGACTTTCCTGTGTGGCCTCTGGAT |
| | nucleic acid sequence | TCACCTTCCATAACTATCACATGAACTGGGTCCGCCAGGCT |
| | | CCAGGGAAGGGACTTCAGTGGGTCGCACACATTAGCAGTG |
| | | ATGGGAGATTCATAAGCTATGCAGACACTGTGAAGGGCCG |
| | | ATTCACCATCTCCAGAGACAACGCCAAGAACACGCTGTATC |
| | | TTCAGATGAACAGCCTGAGAGCCGAGGACACGGCCGTGTA |
| | | TTACTGTGCGAATGGATTGTTTCTGGTACTTGGGGGGGAG |
| | | AACTACTGGGGCCAGGGAACCCTGGTCACCGTCTCCTCAG |
| 122 | PMX023 Heavy chain | EVQLVESGGDLMKPGGSLRLSCVASGFTFHNYHMNWVRQA |
| | variable region amino | PGKGLQWVAHISSDGRFISYADTVKGRFTISRDNAKNTLYLQ |
| | acid sequence | MNSLRAEDTAVYYCANGLFLVLGGENYWGQGTLVTVSS |
| 123 | PMX023 light chain | TCCTATGTGCTGACACAGCTGCCATCCAAAAATGTGACCCT |
| | variable region | GAAGCAGCCGGCCCACATCACCTGTGGGGGAGACAACATT |
| | nucleotide sequence | GGAAGTAAAAGTGTTCACTGGTACCAGCAGAAGCTGGGCC |
| | | AGGCCCCTATATTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGTGGGACAGCAGTG |
| | | CTAGTGTGTTCGGCGGAGGCACCCATCTGACCGTCCTCG |
| 124 | PMX023 light chain | SYVLTQLPSKNVTLKQPAHITCGGDNIGSKSVHWYQQKLGQA |
| | variable region | PILIIYYDSSRPTGIPERFSGANSGNTATLTISGALAEDEADYYC |
| | amino acid sequence | QVWDSSASVFGGGTHLTVL |
| 125 | PMX023 heavy chain | GFTFHNY |
| | CDR1 | |
| 126 | PMX023 heavy chain | SSDGRF |
| | CDR2 | |
| 127 | PMX023 heavy chain | CANGLFLVLGGENYW |
| | CDR3 | |
| 128 | PMX023 light chain | GGDNIGSKSVH |
| | CDR1 | |
| 129 | PMX023 light chain | YDSSRPT |
| | CDR2 | |
| 130 | PMX023 light chain | CQVWDSSASVF |
| | CDR3 | |
| 131 | PMX024 Heavy chain | GAGGTGCTACTGATGGAGTCTGGGGGAGACCTGGTGAAG |
| | variable region | CCTGGGGGGTCCCTGAGACTCTCCTGTGTGGCCTCTGGAT |
| | nucleic acid sequence | TCACCTTCAGTAGCAACTACATGTACTGGATCCGCCAGGCT |
| | | CCAGGGAAGGGGCTGCAGTGGGTCTCACAAATTAGCGGT |
| | | GATGGAAGTTTCACAAACTACGCAGACGCTGTGAAGGGCC |
| | | GATTCACCATCTCCAGAGACAATGCCAAGAACACACTGTAT |
| | | CTCCAGATGAACAGCCTGAGAGATGAGGACACGGCAGTTT |
| | | TTTACTGTGCAAGTGGGATATACCCCAATGCTTTTGGTTAC |
| | | TGGGGCCAGGGCACCCTGGTCACTGTCTCCTCAG |
| 132 | PMX024 Heavy chain | EVLLMESGGDLVKPGGSLRLSCVASGFTFSSNYMYWIRQAP |
| | variable region amino | GKGLQWVSQISGDGSFTNYADAVKGRFTISRDNAKNTLYLQM |
| | acid sequence | NSLRDEDTAVFYCASGIYPNAFGYWGQGTLVTVSS |
| 133 | PMX024 light chain | TCCTATGTGCTGACACAGCCGCCATCCAAAAATGTGACCCT |
| | variable region | GAAGCAGCCGGCCCACATCACCTGTGGCGGAGACAATATT |
| | nucleotide sequence | GGAAGTAAAAGTGTTCACTGGTATCAGCAGAAGCTGGGCC |
| | | AGGCCCCTGTACTGATTATCTATTATGATAGCAGCAGGCCG |
| | | ACAGGGATCCCTGAGCGATTCTCCGGCGCCAACTCGGGG |
| | | AACACGGCCACCCTGACCATCAGCGGGGCCCTGGCCGAG |
| | | GACGAGGCTGACTATTACTGCCAGGTGAGGGACAGCAGTG |
| | | CTAATGTGTTCGGCGGAGGCACCCATCTGACCGTCCTCG |
| 134 | PMX024 light chain | SYVLTQPPSKNVTLKQPAHITCGGDNIGSKSVHWYQQKLGQA |
| | variable region | PVLIIYYDSSRPTGIPERFSGANSGNTATLTISGALAEDEADYY |
| | amino acid sequence | CQVRDSSANVFGGGTHLTVL |
| 135 | PMX024 heavy chain | GFTFSSN |
| | CDR1 | |
| 136 | PMX024 heavy chain | SGDGSF |
| | CDR2 | |
| 137 | PMX024 heavy chain | CASGIYPNAFGYW |
| | CDR3 | |
| 138 | PMX024 light chain | GGDNIGSKSVH |
| | CDR1 | |
| 139 | PMX024 light chain | YDSSRPT |
| | CDR2 | |
| 140 | PMX024 light chain | CQVRDSSANVF |
| | CDR3 | |
| 141 | WT IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEMLGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 142 | Def 1 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NHIGLPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 143 | Def 2 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 144 | Def 3 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 145 | Def 4 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPESLGGPS |
| | | VFIFPPKPKDTLLIARTPEVTCVVVDLGREDPEVQISWFVDGK |
| | | QMQTAKTQPREEQFNGTYRVVSVLPIGHQDWLKGKQFTCKV |
| | | NHIGLGSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 146 | Def 5 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 147 | Def 6 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 148 | Def 7 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPEDPEVQISWFVDGK |
| | | QMQTAKTQPREEQFNGTYRVVSVLPIGHQDWLKGKQFTCKV |
| | | NNKALGSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 149 | Def 8 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPGMLGGPS |
| | | VFIFPPKPKDTLLIARTPEVTCVVVDLDPENPEVQISWFVDGK |
| | | QMQTAKTQPREEQFNGTYRVVSVLPIGHQDWLKGKQFTCKV |
| | | NNKALPSPIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 150 | Def 9 IgGB Constant | ASTTAPSVFPLAPSCGSTSGSTVALACLVSGYFPEPVTVSWN |
| | region | SGSLTSGVHTFPSVLQSSGLYSLSSMVTVPSSRWPSETFTCN |
| | | VAHPASKTKVDKPVPKRENGRVPRPPDCPKCPAPEAAGGPS |
| | | VFIFPPKPKDTLLIARTPEVTCVVVDLDPENPEVQISWFVDGK |
| | | QMQTAKTQPREEQFNGTYRVVSVLPIGHQDWLKGKQFTCKV |
| | | NNKALPSSIERTISKARGQAHQPSVYVLPPSREELSKNTVSLT |
| | | CLIKDFFPPDIDVEWQSNGQQEPESKYRTTPPQLDEDGSYFL |
| | | YSKLSVDKSRWQRGDTFICAVMHEALHNHYTQKSLSHSPGK |
| 151 | Ofa-VH-cIgGB WT | MELGLSWIFLLAILKGVQCEVQLVESGGGLVQPGRSLRLSCA |
| | | ASGFTFNDYAMHWVRQAPGKGLEWVSTISWNSGSIGYADSV |
| | | KGRFTISRDNAKKSLYLQMNSLRAEDTALYYCAKDIQYGNYY |
| | | YGMDVWGQGTTVTVSSASTTAPSVFPLAPSCGSTSGSTVAL |
| | | ACLVSGYFPEPVTVSWNSGSLTSGVHTFPSVLQSSGLYSLSS |
| | | MVTVPSSRWPSETFTCNVAHPASKTKV |
| | | DKPVPKRENGRVPRPPDCPKCPAPEMLGGPSVFIFPPKPKDT |
| | | LLIARTPEVTCVVVDLDPEDPEVQISWFVDGKQMQTAKTQPR |
| | | EEQFNGTYRVVSVLPIGHQDWLKGKQFTCKVNNKALPSPIER |
| | | TISKARGQAHQPSVYVLPPSREELSKNTVSLTCLIKDFFPPDID |
| | | VEWQSNGQQEPESKYRTTPPQLDEDGSYFLYSKLSVDKSRW |
| | | QRGDTFICAVMHEALHNHYTQKSLSHSPGK |
| 152 | hCD20 DNA sequence | 5′ATGACAACACCCAGAAATTCAGTAAATGGGACTTTCCCG |
| | used for generation | GCAGAGCCAATGAAAGGCCCTATTGCTATGCAATCTGGTC |
| | of target cells: | CAAAACCACTCTTCAGGAGGATGTCTTCACTGGTGGGCCC |
| | | CACGCAAAGCTTCTTCATGAGGGAATCTAAGACTTTGGGG |
| | | GCTGTCCAGATTATGAATGGGCTCTTCCACATTGCCCTGG |
| | | GGGGTCTTCTGATGATCCCAGCAGGGATCTATGCACCCAT |
| | | CTGTGTGACTGTGTGGTACCCTCTCTGGGGAGGCATTATG |
| | | TATATTATTTCCGGATCACTCCTGGCAGCAACGGAGAAAAA |
| | | CTCCAGGAAGTGTTTGGTCAAAGGAAAAATGATAATGAATT |
| | | CATTGAGCCTCTTTGCTGCCATTTCTGGAATGATTCTTTCAA |
| | | TCATGGACATACTTAATATTAAAATTTCCCATTTTTTAAAAAT |
| | | GGAGAGTCTGAATTTTATTAGAGCTCACACACCATATATTA |
| | | ACATATACAACTGTGAACCAGCTAATCCCTCTGAGAAAAAC |
| | | TCCCCATCTACCCAATACTGTTACAGCATACAATCTCTGTT |
| | | CTTGGGCATTTTGTCAGTGATGCTGATCTTTGCCTTCTTCC |
| | | AGGAACTTGTAATAGCTGGCATCGTTGAGAATGAATGGAAA |
| | | AGAACGTGCTCCAGACCCAAATCTAACATAGTTCTCCTGTC |
| | | AGCAGAAGAAAAAAAAGAACAGACTATTGAAATAAAAGAAG |
| | | AAGTGGTTGGGCTAACTGAAACATCTTCCCAACCAAAGAAT |
| | | GAAGAAGACATTGAAATTATTCCAATCCAAGAAGAGGAAGA |
| | | AGAAGAAACAGAGACGAACTTTCCAGAACCTCCCCAAGAT |
| | | CAGGAATCCTCACCAATAGAAAATGACAGCTCTCCTTAA3′ |
| 153 | IgG-B | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSTSGSTVALAC |
| | | LVSGYFPEPVTVSWNSGSLTSGVHTFPSVLQSSGLYSLSSMV |
| | | TVPSSRWPSETFTCNVAHPASKTKVDKPVPKRENGRVPRPP |
| | | DCPKCPAPEMLGGPSVFIFPPKPKDTLLIARTPEVTCVVVDLD |
| | | PEDPEVQISWFVDGKQMQTAKTQPREEQFNGTYRVVSVLPI |
| | | GHQDWLKGKQFTCKVNNKALPSPIERTISKARGQAHQPSVYV |
| | | LPPSREELSKNTVSLTCLIKDFFPPDIDVEWQSNGQQEPESKY |
| | | RTTPPQLDEDGSYFLYSKLSVDKSRWQRGDTFICAVMHEALH |
| | | NHYTQKSLSHSPGK |
| 154 | IgG-A | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSTSGSTVALAC |
| | | LVSGYFPEPVTVSWNSGSLTSGVHTFPSVLQSSGLYSLSSMV |
| | | TVPSSRWPSETFTCNVVHPASNTKVDKPVFNECRCTDTPPC |
| | | PVPEPLGGPSVLIFPPKPKDILRITRTPEVTCVVLDLGREDPEV |
| | | QISWFVDGKEVHTAKTQSREQQFNGTYRVVSVLPIEHQDWLT |
| | | GKEFKCRVNHIDLPSPIERTISKARGRAHKPSVYVLPPSPKEL |
| | | SSSDTVSITCLIKDFYPPDIDVEWQSNGQQEPERKHRMTPPQ |
| | | LDEDGSYFLYSKLSVDKSRWQQGDPFTCAVMHETLQNHYTD |
| | | LSLSHSPGK |
| 155 | IgG-C | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASTTAPSVFPLAPSCGSQSGSTVALAC |
| | | LVSGYIPEPVTVSWNSGSLTSGVHTFPSILQSSGLYSLSSMVT |
| | | VPSSRWPSETFTCNVAHPATNTKVDKPVVKECECKCNCNNC |
| | | PCPGCGLLGGPSVFIFPPKPKDILVTARTPTVTCVVVDLDPEN |
| | | PEVQISWFVDSKQVQTANTQPREEQSNGTYRVVSVLPIGHQ |
| | | DWLSGKQFKCKVNNKALPSPIEEIISKTPGQAHQPNVYVLPPS |
| | | RDEMSKNTVTLTCLVKDFFPPEIDVEWQSNGQQEPESKYRM |
| | | TPPQLDEDGSYFLYSKLSVDKSRWQRGDTFICAVMHEALHN |
| | | HYTQKSLSHSPGK |
| 156 | IgG-D | MEFVLGWVFLVAILQGVQGEVQLVESGGDLVKPAGSLRLSCV |
| | | ASGFTFSNNAMNWVRQAPGKGLQWVAGINSGGSTASADAV |
| | | KGRFTISRDNAKNTVYLQMNSLTAEDTAVYYCAKVIGNWIATS |
| | | DLDYWGQGTLVIVSSASSTAPSVFPLAPSCGSTSGSTVALAC |
| | | LVSGYFPEPVTVSWNSGSLTSGVHTFPSVLKSSGLYSLSSMV |
| | | TVPSSRLPSETFTCNVVHPATNTKVDKPVPKESTCKCISPCPV |
| | | PESLGGPSVFIFPPKPKDILRITRTPEVTCVVLDLGREDPEVQI |
| | | SWFVDGKEVHTAKTQPREQQFNSTYRVVSVLPIEHQDWLTG |
| | | KEFKCRVNHIGLPSPIERTISKARGQAHQPGVYVLPPSPKELS |
| | | SSDTVTLTCLIKDFFPPEIDVEWQSNGQPEPESKYHTTAPQLD |
| | | EDGSYFLYSKLSVDKSRWQQGDPFTCAVMHEALQNHYTDLS |
| | | LSHSPGK |

TABLE 3

| | Cell binding | SPR K_{D} exp 1 | SPR K_{D} exp 2 |
|---|---|---|---|
| Candidate | K_{D} (M) | (M) | (M) |
| PMX 012 | 3.70e−8 | 1.48e−8 | 1.48e−8 |
| PMX 014 | 7.46e−8 | 1.97e−8 | 6.61e−9 |
| PMX 023 | 3.99e−8 | 2.84e−8 | 7.53e−8 |

TABLE 4

| Candidates | IC50 |
|---|---|
| PMX012 | 7.41e−8 |
| PMX014 | 2.26e−8 |
| PMX020 | 2.01e−8 |
| PMX023 | 2.82e−9 |

TABLE 5

| Candidates | Overall identity (%) | Average pair identity (%) |
|---|---|---|
| All | 29.20 | 76.60 |
| Group 1 | 82.5 | 89.72 |
| Group 2 | 89.26 | 92.84 |
| Group 3 | 92.37 | 92.37 |

TABLE 6

| Candidates | Overall identity (%) | Average pair identity (%) |
|---|---|---|
| All | 81.65 | 96.38 |
| Group 1 | 95.37 | 98.43 |
| Group 2 | 97.22 | 98.15 |
| Group 3 | 97.17 | 97.17 |

1. An antibody or fragment thereof that specifically binds to companion animal OX40L.

2. The antibody or fragment according to claim 1 wherein said companion animal is a dog or a cat.

3. The antibody or fragment according to claim 1 or 2 wherein the antibody or fragment binds to canine OX40L.

4. The antibody or fragment according to claim 3 wherein said antibody or fragment is capable of
a) reducing, inhibiting or neutralising OX40 activity or activation in the companion animal or in a cell of the companion animal;
b) modifying secretion of a cytokine in the companion animal or in a cell of the companion animal and/or
c) decreasing proliferation of leukocytes in the companion animal.

5. The antibody or fragment according to a preceding claim wherein the antibody is a canine or caninized antibody.

6. The antibody or fragment according to claim 5 wherein the antibody is selected from one of the following antibodies:
an antibody comprising a HC CDR1 comprising SEQ ID No: 15 or a sequence with at least 80% sequence identity thereto, a HC CDR2 comprising SEQ ID No: 16 or a sequence with at least 80% sequence identity thereto, a HC CDR3 comprising SEQ ID No: 17, or a sequence with at least 80% sequence identity thereto, a LC CDR1 comprising SEQ ID No: 18 or a sequence with at least 80% sequence identity thereto, a LC CDR2 comprising SEQ ID No: 19 or a sequence with at least 80% sequence identity thereto and a LC CDR3 comprising SEQ ID No: 20 or a sequence with at least 80% sequence identity thereto;
an antibody comprising a HC CDR1 comprising SEQ ID No: 25 or a sequence with at least 80% sequence identity thereto, a HC CDR2 comprising SEQ ID No: 26 or a sequence with at least 80% sequence identity thereto, a HC CDR3 comprising SEQ ID No: 27, or a sequence with at least 80% sequence identity thereto, a LC CDR1 comprising SEQ ID No: 28 or a sequence with at least 80% sequence identity thereto, a LC CDR2 comprising SEQ ID No: 29 or a sequence with at least 80% sequence identity thereto and a LC CDR3 comprising SEQ ID No: 30 or a sequence with at least 80% sequence identity thereto;
an antibody comprising a HC CDR1 comprising SEQ ID No: 35 or a sequence with at least 80% sequence identity thereto, a HC CDR2 comprising SEQ ID No: 36 or a sequence with at least 80% sequence identity thereto, a HC CDR3 comprising SEQ ID No: 37, or a sequence with at least 80% sequence identity thereto, a LC CDR1 comprising SEQ ID No: 38 or a sequence with at least 80% sequence identity thereto, a LC CDR2 comprising SEQ ID No: 39 or a sequence with at least 80% sequence identity thereto and a LC CDR3 comprising SEQ ID No: 40 or a sequence with at least 80% sequence identity thereto;
an antibody comprising a HC CDR1 comprising SEQ ID No: 45 or a sequence with at least 80% sequence identity thereto, a HC CDR2 comprising SEQ ID No: 46 or a sequence with at least 80% sequence identity thereto, a HC CDR3 comprising SEQ ID No: 47, or a sequence with at least 80% sequence identity thereto, a LC CDR1 comprising SEQ ID No: 48 or a sequence with at least 80% sequence identity thereto, a LC CDR2 comprising SEQ ID No: 49 or a sequence with at least 80% sequence identity thereto and a LC CDR3 comprising SEQ ID No: 50 or a sequence with at least 80% sequence identity thereto;
an antibody comprising a HC CDR1 comprising SEQ ID No: 55 or a sequence with at least 80% sequence identity thereto, a HC CDR2 comprising SEQ ID No: 56 or a sequence with at least 80% sequence identity thereto, a HC CDR3 comprising SEQ ID No: 57, or a sequence with at least 80% sequence identity thereto, a LC CDR1 comprising SEQ ID No: 58 or a sequence with at least 80% sequence identity thereto, a LC CDR2 comprising SEQ ID No: 59 or a sequence with at least 80% sequence identity thereto and a LC CDR3 comprising SEQ ID No: 60 or a sequence with at least 80% sequence identity thereto;
an antibody comprising a HC CDR1 comprising SEQ ID No: 65 or a sequence with at least 80% sequence identity thereto, a HC CDR2 comprising SEQ ID No: 66 or a sequence with at least 80% sequence identity thereto, a HC CDR3 comprising SEQ ID No: 67, or a sequence with at least 80% sequence identity thereto, a LC CDR1 comprising SEQ ID No: 68 or a sequence with at least 80% sequence identity thereto, a LC CDR2 comprising SEQ ID No: 69 or a sequence with at least 80% sequence identity thereto and a LC CDR3 comprising SEQ ID No: 70 or a sequence with at least 80% sequence identity thereto;
an antibody comprising a HC CDR1 comprising SEQ ID No: 75 or a sequence with at least 80% sequence identity thereto, a HC CDR2 comprising SEQ ID No: 76 or a sequence with at least 80% sequence identity thereto, a HC CDR3 comprising SEQ ID No: 77, or a sequence with at least 80% sequence identity thereto, a LC CDR1 comprising SEQ ID No: 78 or a sequence with at least 80% sequence identity thereto, a LC CDR2 comprising SEQ ID No: 79 or a sequence with at least 80% sequence identity thereto and a LC CDR3 comprising SEQ ID No: 80 or a sequence with at least 80% sequence identity thereto;
an antibody comprising a HC CDR1 comprising SEQ ID No: 85 or a sequence with at least 80% sequence identity thereto, a HC CDR2 comprising SEQ ID No: 86 or a sequence with at least 80% sequence identity thereto, a HC CDR3 comprising SEQ ID No: 87, or a sequence with at least 80% sequence identity thereto, a LC CDR1 comprising SEQ ID No: 88 or a sequence with at least 80% sequence identity thereto, a LC CDR2 comprising SEQ ID No: 89 or a sequence with at least 80% sequence identity thereto and a LC CDR3 comprising SEQ ID No: 90 or a sequence with at least 80% sequence identity thereto;
an antibody comprising a HC CDR1 comprising SEQ ID No: 95 or a sequence with at least 80% sequence identity thereto, a HC CDR2 comprising SEQ ID No: 96 or a sequence with at least 80% sequence identity thereto, a HC CDR3 comprising SEQ ID No: 97, or a sequence with at least 80% sequence identity thereto, a LC CDR1 comprising SEQ ID No: 98 or a sequence with at least 80% sequence identity thereto, a LC CDR2 comprising SEQ ID No: 99 or a sequence with at least 80% sequence identity thereto and a LC CDR3 comprising SEQ ID No: 100 or a sequence with at least 80% sequence identity thereto;
an antibody comprising a HC CDR1 comprising SEQ ID No: 105 or a sequence with at least 80% sequence identity thereto, a HC CDR2 comprising SEQ ID No: 106 or a sequence with at least 80% sequence identity thereto, a HC CDR3 comprising SEQ ID No: 107, or a sequence with at least 80% sequence identity thereto, a LC CDR1 comprising SEQ ID No: 108 or a sequence with at least 80% sequence identity thereto, a LC CDR2 comprising SEQ ID No: 109 or a sequence with at least 80% sequence identity thereto and a LC CDR3 comprising SEQ ID No: 110 or a sequence with at least 80% sequence identity thereto;
an antibody comprising a HC CDR1 comprising SEQ ID No: 115 or a sequence with at least 80% sequence identity thereto, a HC CDR2 comprising SEQ ID No: 116 or a sequence with at least 80% sequence identity thereto, a HC CDR3 comprising SEQ ID No: 117, or a sequence with at least 80% sequence identity thereto, a LC CDR1 comprising SEQ ID No: 118 or a sequence with at least 80% sequence identity thereto, a LC CDR2 comprising SEQ ID No: 119 or a sequence with at least 80% sequence identity thereto and a LC CDR3 comprising SEQ ID No: 120 or a sequence with at least 80% sequence identity thereto;
an antibody comprising a HC CDR1 comprising SEQ ID No: 125 or a sequence with at least 80% sequence identity thereto, a HC CDR2 comprising SEQ ID No: 126 or a sequence with at least 80% sequence identity thereto, a HC CDR3 comprising SEQ ID No: 127, or a sequence with at least 80% sequence identity thereto, a LC CDR1 comprising SEQ ID No: 128 or a sequence with at least 80% sequence identity thereto, a LC CDR2 comprising SEQ ID No: 129 or a sequence with at least 80% sequence identity thereto and a LC CDR3 comprising SEQ ID No: 130 or a sequence with at least 80% sequence identity thereto; or
an antibody comprising a HC CDR1 comprising SEQ ID No: 135 or a sequence with at least 80% sequence identity thereto, a HC CDR2 comprising SEQ ID No: 136 or a sequence with at least 80% sequence identity thereto, a HC CDR3 comprising SEQ ID No: 137, or a sequence with at least 80% sequence identity thereto, a LC CDR1 comprising SEQ ID No: 138 or a sequence with at least 80% sequence identity thereto, a LC CDR2 comprising SEQ ID No: 139 or a sequence with at least 80% sequence identity thereto and a LC CDR3 comprising SEQ ID No: 140 or a sequence with at least 80% sequence identity thereto.

7. The antibody or antibody fragment according to claim 5 or 6 wherein the antibody is selected from one of the following antibodies:
an antibody comprising a HC variable region comprising or consisting of SEQ ID No. 12 or a sequence with at least 80% sequence identity thereto and a LC variable region comprising or consisting of SEQ ID No. 14 or a sequence with at least 80% sequence identity thereto;
an antibody comprising a HC variable region comprising or consisting of SEQ ID No. 22 or a sequence with at least 80% sequence identity thereto and a LC variable region comprising or consisting of SEQ ID No. 24 or a sequence with at least 80% sequence identity thereto;
an antibody comprising a HC variable region comprising or consisting of SEQ ID No. 32 or a sequence with at least 80% sequence identity thereto and a LC variable region comprising or consisting of SEQ ID No. 34 or a sequence with at least 80% sequence identity thereto;
an antibody comprising a HC variable region comprising or consisting of SEQ ID No. 42 or a sequence with at least 80% sequence identity thereto and a LC variable region comprising or consisting of SEQ ID No. 44 or a sequence with at least 80% sequence identity thereto;
an antibody comprising a HC variable region comprising or consisting of SEQ ID No. 52 or a sequence with at least 80% sequence identity thereto and a LC variable region comprising or consisting of SEQ ID No. 54 or a sequence with at least 80% sequence identity thereto;
an antibody comprising a HC variable region comprising or consisting of SEQ ID No. 62 or a sequence with at least 80% sequence identity thereto and a LC variable region comprising or consisting of SEQ ID No. 64 or a sequence with at least 80% sequence identity thereto;
an antibody comprising a HC variable region comprising or consisting of SEQ ID No. 72 or a sequence with at least 80% sequence identity thereto and a LC variable region comprising or consisting of SEQ ID No. 74 or a sequence with at least 80% sequence identity thereto;
an antibody comprising a HC variable region comprising or consisting of SEQ ID No. 82 or a sequence with at least 80% sequence identity thereto and a LC variable region comprising or consisting of SEQ ID No. 84 or a sequence with at least 80% sequence identity thereto;
an antibody comprising a HC variable region comprising or consisting of SEQ ID No. 92 or a sequence with at least 80% sequence identity thereto and a LC variable region comprising or consisting of SEQ ID No. 94 or a sequence with at least 80% sequence identity thereto;
an antibody comprising a HC variable region comprising or consisting of SEQ ID No. 102 or a sequence with at least 80% sequence identity thereto and a LC variable region comprising or consisting of SEQ ID No. 104 or a sequence with at least 80% sequence identity thereto;
an antibody comprising a HC variable region comprising or consisting of SEQ ID No. 112 or a sequence with at least 80% sequence identity thereto and a LC variable region comprising or consisting of SEQ ID No. 114 or a sequence with at least 80% sequence identity thereto;
an antibody comprising a HC variable region comprising or consisting of SEQ ID No. 122 or a sequence with at least 80% sequence identity thereto and a LC variable region comprising or consisting of SEQ ID No. 124 or a sequence with at least 80% sequence identity thereto; or
an antibody comprising a HC variable region comprising or consisting of SEQ ID No. 132 or a sequence with at least 80% sequence identity thereto and a LC variable region comprising or consisting of SEQ ID No. 134 or a sequence with at least 80% sequence identity thereto.

8. The antibody or fragment according to a preceding claim wherein said fragment is selected from a F(ab′)2, Fab, Fv, scFv, heavy chain, light chain, variable heavy (VH), variable light (VL) chain, CDR region, single VH or VL domain, maxibodies, minibodies, intrabodies, diabodies, triabodies, tetrabodies, and bis-scFv, and polypeptides that contain at least a portion of an immunoglobulin that is sufficient to confer specific antigen binding to the polypeptide.

9. The antibody or fragment according to a preceding claim wherein said antibody or fragment is conjugated to another moiety.

10. The antibody or fragment according to a preceding claim comprising a therapeutic moiety, half life extending moiety or label.

11. A binding molecule comprising an antibody or fragment according to a preceding claim.

12. The antibody or fragment according to any of claims 1 to 10 or the binding molecule of claim 11 for use in the treatment of a disease.

13. A pharmaceutical composition comprising an antibody or fragment thereof according to any of claims 1 to 10 or the binding molecule of claim 11.

14. An antibody or fragment thereof according to any of claims 1 to 10, the binding molecule of claim 11 or a pharmaceutical composition according to claim 13 for use in the treatment of an OX40 or OX40L-mediated disease.

15. A method of treating or preventing an OX40 or OX40L-mediated disease comprising administering to a subject in need thereof an antibody or fragment according to any of claims 1 to 10, the binding molecule of claim 11 or the pharmaceutical composition according to claim 13.

16. The antibody or fragment, binding molecule or pharmaceutical composition according claim 14 or the method of claim 15 wherein said disease is selected from an inflammatory or autoimmune disease.

17. The antibody or fragment, binding molecule, pharmaceutical composition or method according claim 16 wherein said disease is selected from an inflammatory skin diseases, including atopic dermatitis, allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma, bacterial pneumonia, antigen-antibody complex mediated diseases; inflammations of the lung, including pleurisy, alveolitis, vasculitis, pneumonia, chronic bronchitis, bronchiectasis, and cystic fibrosis.

18. The antibody or fragment, binding molecule, pharmaceutical composition according to any of claims 14, 16 or 17 or the method according to any of claims 15 to 17 wherein said antibody or fragment is administered together with one or more therapeutic agent.

19. The antibody or fragment, binding molecule, pharmaceutical composition or method according claim 18 wherein said one or more therapeutic agent is selected from rapamycin (sirolimus), tacrolimus, cyclosporine (e.g. Atopica®), corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, PDE4 inhibitors, lokietmab (Cytopoint®), and oclacitinib (Apoquel®).

20. A method of decreasing the secretion of cytokines comprising administering to a subject in need thereof an antibody or fragment according to any of claims 1 to 10, a binding molecule of claim 11 or a pharmaceutical composition of claim 13.

21. A multispecific binding agent comprising an antibody or fragment thereof according to any of claims 1 to 10 or a binding molecule of claim 11.

22. A combination therapy comprising antibody or fragment according to any of claims 1 to 10, a binding molecule of claim 11 or a pharmaceutical composition of claim 13.

23. An immunoconjugate comprising an antibody or fragment according to any of claims 1 to 10 or a binding molecule of claim 11.

24. A kit comprising an antibody or fragment thereof according to any of claims 1 to 10, the binding molecule of claim 11 or a pharmaceutical composition according to claim 13.

25. An isolated canine OX40 protein comprising SEQ ID NO. 4 or 6 or a variant thereof.

26. An isolated nucleic acid molecule encoding a protein according to claim 25, optionally comprising SEQ ID NO. 3 or 5 or a variant thereof.

27. A method for detecting OX40L or OX40 in a companion animal comprising contacting a test sample with an antibody or fragment according to any of claims 1 to 10 or a binding molecule of claim 11.


SEQUENCE LISTING (160 sequences)

SEQ ID NO: 1 - protein, 183 aa, Canis familiaris
MEGVQPLDQNVGNTPGRRFQKNKVLLVAAIIQGLGLLLCFTYICLHFYASQVPPQYPPIQSIRVQFTRCENEKGCIITSPSKDETMKVQDNSIIINCDGFYLISLKGYFSEELSLSLYYRKGRGPLFSLSKVTSVDSIGVAYLAFKDKVYFNVTTHSTSYKDIQVNGGELILIHQNPGGFCAY

SEQ ID NO: 2 - DNA, 552 bp, Canis familiaris
atggaaggggtccaacccctggaccagaatgtgggaaacacaccagggcgaagattccagaagaacaaggtattgctggtggcagccataattcaggggctgggtctgctcctgtgtttcacctacatctgcctgcacttctatgcttctcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctactga

SEQ ID NO: 3 - DNA, 708 bp, Canis familiaris
atgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggtggaaatagcttccggacccccatccaagaggagcatgctgacgccaactccaccctggccaagatctga

SEQ ID NO: 4 - protein, 235 aa, Canis familiaris
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTNCTLMGRRTMQPASKSSDAVCEDRSLPATLPWETQSPLTRPPTPQPTMAWPRTSQGPFTPPTEPPRGGNSFRTPIQEEHADANSTLAKI

SEQ ID NO: 5 - DNA, 813 bp, Canis familiaris
atgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggcccccagctggctgctgtcctgggcttgggcctaggcttgctggcccccgtggcagccgcactggccttgctcctgcaccacagagcctggcggctgccccccggtggaaatagcttccggacccccatccaagaggagcatgctgacgccaactccaccctggccaagatctga

SEQ ID NO: 6 - protein, 270 aa, Canis familiaris
MRMFVESLRLSGPHSALLLLGLVLGAVAEHNCFGNTYPKDGKCCNDCPPGYGMESRCSRSHDTKCHQCPSGFYNEATNYEPCKPCTQCNQRSGSEPKRRCTPTQDTICSCKPGTEPRDGYKRGVDCAPCPPGHFSPGDDQACKPWTNCTLMGRRTMQPASKSSDAVCEDRSLPATLPWETQSPLTRPPTPQPTMAWPRTSQGPFTPPTEPPRGPQLAAVLGLGLGLLAPVAAALALLLHHRAWRLPPGGNSFRTPIQEEHADANSTLAKI

SEQ ID NO: 7 - DNA, 20 bp, Artificial Sequence
  OX40 Forward primer
ggcagagatgaggatgttcg

SEQ ID NO: 8 - DNA, 21 bp, Artificial Sequence
  OX40 Reverse primer
cactggctgctcagatcttgg

SEQ ID NO: 9 - DNA, 20 bp, Artificial Sequence
  OX40L Forward
gccacagttttcatctccct

SEQ ID NO: 10 - DNA, 18 bp, Artificial Sequence
  OX40L Reverse
gcttggcttaggtgcagc

SEQ ID NO: 11 - DNA, 364 bp, Canis familiaris
gaggtgcagctggtggagtctgggggagacctggtgaagcctggggggtccctgagacttgcctgtgtgacctctggattcaccttcagtagctatcacatgaactgggtccgccaggctccagggaaggggcttcagtgggtcgcttacattaacactggtggaactgtcacaacctatgcagacgctatgaatgcagacgctgtgaggggccgattcaccatctccagagacaacgtcaagaacacgctgtatcttcagatgaatagactgagagccgaggacacggccgtatattactgtgcgcgcgggtatggggtctttgactactggggccagggaaccctggtcaccgtctccccgg

SEQ ID NO: 12 - protein, 121 aa, Canis familiaris
EVQLVESGGDLVKPGGSLRLACVTSGFTFSSYHMNWVRQAPGKGLQWVAYINTGGTVTTYADAMNADAVRGRFTISRDNVKNTLYLQMNRLRAEDTAVYYCARGYGVFDYWGQGTLVTVSP

SEQ ID NO: 13 - DNA, 328 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgtaggccgacagggatccctgagcgattctccggcgccaactcggggaacacggccaccctgaccatcagcggggccctggccgaggacgaggctgactattactgccaggtgtgggacaacagtgatagggctagttgggtattcggtgaagggacccagctgaccgtcctcg

SEQ ID NO: 14 - protein, 109 aa, Canis familiaris
SYVLTQLPSKNVTLKQPAHITCGGDNIGSKSVHWYQQKLGQAPVLIIYYDSRRPTGIPERFSGANSGNTATLTISGALAEDEADYYCQVWDNSDRASWVFGEGTQLTVL

SEQ ID NO: 15 - protein, 7 aa, Canis familiaris
GFTFSSY

SEQ ID NO: 16 - protein, 6 aa, Canis familiaris
NTGGTV

SEQ ID NO: 17 - protein, 11 aa, Canis familiaris [sequence omitted]

SEQ ID NO: 18 - protein, 11 aa, Canis familiaris
GGDNIGSKSVH

SEQ ID NO: 19 - protein, 7 aa, Canis familiaris
YDSRRPT

SEQ ID NO: 20 - protein, 14 aa, Canis familiaris
CQVWDNSDRASWVF

SEQ ID NO: 21 - DNA, 370 bp, Canis familiaris
gaactcacactgcaggagtcagggccaggactggtgaagccctcacagaccctctctctcacctgtgttgtgtccggaggctccgtcaccagcagtcactactggaactggatccgccagcgccctgggaggggactggaatggatggggtcctggacaggcggaacaaactacaacccggcattccagggacgcatctctgtcacttctgacacggccaagaaccaattctccctgcaactgagttccttgaccaccgaggacacggccgtgtattattgtgcacgaggaggcggatatagtggcacctggaaggattactatgttatggactactggggccatggcacctcagtcatcgtgtcctcag

SEQ ID NO: 22 - protein, 123 aa, Canis familiaris
ELTLQESGPGLVKPSQTLSLTCVVSGGSVTSSHYWNWIRQRPGRGLEWMGSWTGGTNYNPAFQGRISVTSDTAKNQFSLQLSSLTTEDTAVYYCARGGGYSGTWKDYYVMDYWGHGTSVIVSS

SEQ ID NO: 23 - DNA, 325 bp, Canis familiaris
tcctatgtgctgacacagctgccatccaaaaatgtgaccctgaaccagccggcccacatcacctgtgggggagacaaccttggaagtaaaagtgttcactggtaccagcagaagctgggccaggcccctgtactgattatctattttgataccagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 24 - protein, 108 aa, Canis familiaris
SYVLTQLPSKNVTLNQPAHITCGGDNLGSKSVHWYQQKLGQAPVLIIYFDTSRPTGIPERFSGANSGNTATLTISGALAEDEADYYCQVWDSSAKASVFGGGTHLTVL

SEQ ID NO: 25 - protein, 8 aa, Canis familiaris
GGSVTSSH

SEQ ID NO: 26 - protein, 3 aa, Canis familiaris
TGG

SEQ ID NO: 27 - protein, 20 aa, Canis familiaris
CARGGGYSGTWKDYYVMDYW

SEQ ID NO: 28 - protein, 11 aa, Canis familiaris
GGDNLGSKSVH

SEQ ID NO: 29 - protein, 7 aa, Canis familiaris
FDTSRPT

SEQ ID NO: 30 - protein, 13 aa, Canis familiaris
CQVWDSSAKASVF

SEQ ID NO: 31 - DNA, 367 bp, Canis familiaris
gaggtgcagctggtggagtctgggggagacctggtgaagcctggggggtccctgagactttcctgtgtggcctctggattcacattcagtaacttccacatgagttgggtccgccaggctccagggaaggggcttcagtgggtcgcatacattaacagtggtggatttaacataaattatgaagacgctgtgaggggccgcttcaccatctccagagacaacgccaagaacacgttgtatcttcagatgaacagcctgagagccgaagacacggccatttattactgtgcgcgtgattgggatacacatttggatacgaactggttctactactggggccaagggaccctggtcactgtgtcctcag

SEQ ID NO: 32 - protein, 122 aa, Canis familiaris
EVQLVESGGDLVKPGGSLRLSCVASGFTFSNFHMSWVRQAPGKGLQWVAYINSGGFNINYEDAVRGRFTISRDNAKNTLYLQMNSLRAEDTAIYYCARDWDTHLDTNWFYYWGQGTLVTVSS

SEQ ID NO: 33 - DNA, 325 bp, Canis familiaris
tcctatgtgctgacacagctgccatccaaaaatgtgaccctgaagcagtcggcccacatcacctgtcggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacatcagtgctaaggctagtgtgttcggcggaggcacccatctgaccgtcctcg

SEQ ID NO: 34 - protein, 108 aa, Canis familiaris
SYVLTQLPSKNVTLKQSAHITCRGDNIGSKSVHWYQQKLGQAPVLIIYYDSSRPTGIPERFSGANSGNTATLTISGALAEDEADYYCQVWDISAKASVFGGGTHLTVL

SEQ ID NO: 35 - protein, 7 aa, Canis familiaris
GFTFSNF

SEQ ID NO: 36 - protein, 6 aa, Canis familiaris
NSGGFN

SEQ ID NO: 37 - protein, 17 aa, Canis familiaris
CARDWDTHLDTNWFYYW

SEQ ID NO: 38 - protein, 11 aa, Canis familiaris
RGDNIGSKSVH

SEQ ID NO: 39 - protein, 7 aa, Canis familiaris
YDSSRPT

SEQ ID NO: 40 - protein, 13 aa, Canis familiaris
CQVWDISAKASVF

SEQ ID NO: 41 - DNA, 367 bp, Canis familiaris
gaggtgcagctggtggagtctgggggagacctggtgaagcctggggggtccctgagaatttcctgtgtggcctctggattcaccttcagttactaccacatgagctgggtccgccaggctccagggaaggggcttcagtgggtcgcatatatcaacagtggtggatttagtacaaactatgcagacgctgtgaagggccgatgctccatctccagagacaatggcaagaacacgctgtatcttcagatgaacagcctgagacccgaggacacgggcgtttattattgtgcgagtgaaagtcgttggggggattcttacagtggtatgacctactggggccatggcacttcactcttcgtgtcctcag

SEQ ID NO: 42 - protein, 122 aa, Canis familiaris
EVQLVESGGDLVKPGGSLRISCVASGFTFSYYHMSWVRQAPGKGLQWVAYINSGGFSTNYADAVKGRCSISRDNGKNTLYLQMNSLRPEDTGVYYCASESRWGDSYSGMTYWGHGTSLFVSS

SEQ ID NO: 43 - DNA, 325 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgttctgattatctattatgataacagcaggccgacagggatccctgagcgattctccggcgccaagtcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 44 - protein, 108 aa, Canis familiaris
SYVLTQLPSKNVTLKQPAHITCGGDNIGSKSVHWYQQKLGQAPVLIIYYDNSRPTGIPERFSGAKSGNTATLTISGALAEDEADYYCQVWDSSAKASVFGGGTHLTVL

SEQ ID NO: 45 - protein, 7 aa, Canis familiaris
GFTFSYY

SEQ ID NO: 46 - protein, 6 aa, Canis familiaris
NSGGFS

SEQ ID NO: 47 - protein, 17 aa, Canis familiaris
CASESRWGDSYSGMTYW

SEQ ID NO: 48 - protein, 11 aa, Canis familiaris
GGDNIGSKSVH

SEQ ID NO: 49 - protein, 7 aa, Canis familiaris
YDNSRPT

SEQ ID NO: 50 - protein, 13 aa, Canis familiaris
CQVWDSSAKASVF

SEQ ID NO: 51 - DNA, 361 bp, Canis familiaris
gaggtgcagctggtggagtctgggggagacctggtgaagccgggggggtccctgagactttcctgtgtggcctctggattcaccttcagtaattaccacatgaactgggtccgccaggctccagggaaggggcttcagtgggtcgcatacattaccagtgatggaattgtttcaagctacgcagacgttgtgaagggccgattcaccatctccagagacaacgccaagaacacgctttttcttcagatgaacagcctgagagccgaggacacggccgtgtattattgtgcgagtgggttgtttttagtagttgggggggggaccttctggggccagggaaccctggtcaccgtctcctcag

SEQ ID NO: 52 - protein, 120 aa, Canis familiaris
EVQLVESGGDLVKPGGSLRLSCVASGFTFSNYHMNWVRQAPGKGLQWVAYITSDGIVSSYADVVKGRFTISRDNAKNTLFLQMNSLRAEDTAVYYCASGLFLVVGGGTFWGQGTLVTVSS

SEQ ID NO: 53 - DNA, 325 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattctgatagtagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 54 - protein, 108 aa, Canis familiaris
SYVLTQLPSKNVTLKQPAHITCGGDNIGSKSVHWYQQKLGQAPVLIIYSDSSRPTGIPERFSGANSGNTATLTISGALAEDEADYYCQVWDSSAKASVFGGGTHLTVL

SEQ ID NO: 55 - protein, 7 aa, Canis familiaris
GFTFSNY

SEQ ID NO: 56 - protein, 6 aa, Canis familiaris
TSDGIV

SEQ ID NO: 57 - protein, 15 aa, Canis familiaris
CASGLFLVVGGGTFW

SEQ ID NO: 58 - protein, 11 aa, Canis familiaris
GGDNIGSKSVH

SEQ ID NO: 59 - protein, 7 aa, Canis familiaris
SDSSRPT

SEQ ID NO: 60 - protein, 13 aa, Canis familiaris
CQVWDSSAKASVF

SEQ ID NO: 61 - DNA, 364 bp, Canis familiaris
gaggtgcaactggtggagtctgggggagaccttgtgaagcctggggggtccctgagactttcctgtgtggcctctggattcaccttcagtagttaccacatgagctgggtccgccaggctccagggaaggggcttcagtgggtcgcatacattgccagtggtggtactgtcacaacctatgcagacgctgtgaggggccgattcaccatctccagagacaacgccaagaatatgttgtatcttcagatgaacagcctgagagccgaggactcggccgtatattactgtacgaggtggaagggtgggacttttggctatggtatggactactggggccatggcacctcactcttcgtgtcctcag

SEQ ID NO: 62 - protein, 121 aa, Canis familiaris
EVQLVESGGDLVKPGGSLRLSCVASGFTFSSYHMSWVRQAPGKGLQWVAYIASGGTVTTYADAVRGRFTISRDNAKNMLYLQMNSLRAEDSAVYYCTRWKGGTFGYGMDYWGHGTSLFVSS

SEQ ID NO: 63 - DNA, 325 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgataacaacaggccggcagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 64 - protein, 108 aa, Canis familiaris
SYVLTQLPSKNVTLKQPAHITCGGDNIGSKSVHWYQQKLGQAPVLIIYYDNNRPAGIPERFSGANSGNTATLTISGALAEDEADYYCQVWDSSAKASVFGGGTHLTVL

SEQ ID NO: 65 - protein, 7 aa, Canis familiaris
GFTFSSY

SEQ ID NO: 66 - protein, 6 aa, Canis familiaris
ASGGTV

SEQ ID NO: 67 - protein, 16 aa, Canis familiaris
CTRWKGGTFGYGMDYW

SEQ ID NO: 68 - protein, 11 aa, Canis familiaris
GGDNIGSKSVH

SEQ ID NO: 69 - protein, 7 aa, Canis familiaris
YDNNRPA

SEQ ID NO: 70 - protein, 13 aa, Canis familiaris
CQVWDSSAKASVF

SEQ ID NO: 71 - DNA, 361 bp, Canis familiaris
gaggtgcagctggtggagtctgggggagacctggtgaagcctggggggtccctgagactttcctgtgtggcctctggattcaccttcagtaactaccacatgaactgggtccgccaggctccagggaaggggcttcagtgggtcgcatacattaccaatgatggaattgtttcaagctacgcagacgctgtgaagggccgattcaccatctccagagacaacgccaagaacacgctttatcttcagatgaacagcctgagagtcgaggacacggccgtgtattactgtgcgagtggattgtttctagtagttgggggggggaccttctggggccagggaaccctggtcaccgtctcctcag

SEQ ID NO: 72 - protein, 120 aa, Canis familiaris
EVQLVESGGDLVKPGGSLRLSCVASGFTFSNYHMNWVRQAPGKGLQWVAYITNDGIVSSYADAVKGRFTISRDNAKNTLYLQMNSLRVEDTAVYYCASGLFLVVGGGTFWGQGTLVTVSS

SEQ ID NO: 73 - DNA, 325 bp, Canis familiaris
tcctatgtgctgacacagctgccatccaaaaatgtgaccctgaagcagccggcccacatcacctgtgggggagacaacattggaagtaaaagtgttcactggtaccagcataagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 74 - protein, 108 aa, Canis familiaris
SYVLTQLPSKNVTLKQPAHITCGGDNIGSKSVHWYQHKLGQAPVLIIYYDSSRPTGIPERFSGANSGNTATLTISGALAEDEADYYCQVWDSSAKASVFGGGTHLTVL

SEQ ID NO: 75 - protein, 7 aa, Canis familiaris
GFTFSNY

SEQ ID NO: 76 - protein, 6 aa, Canis familiaris
TNDGIV

SEQ ID NO: 77 - protein, 15 aa, Canis familiaris
CASGLFLVVGGGTFW

SEQ ID NO: 78 - protein, 11 aa, Canis familiaris
GGDNIGSKSVH

SEQ ID NO: 79 - protein, 7 aa, Canis familiaris
YDSSRPT

SEQ ID NO: 80 - protein, 13 aa, Canis familiaris
CQVWDSSAKASVF

SEQ ID NO: 81 - DNA, 355 bp, Canis familiaris
gaggtacaactggtggagtcggggggagacctggtgaagcctggggggtccctgagactctcctgtgtggcctctggattcaccttcagtagcaactacatgagctggatccgccaggctccagggaaggggctgcagtgggtctcacaaattagcggtgatggaatttacacaaactacgcagacgctatgaagggccgattcaccatctccagagacaatgccaagaacacgctgtatctgcagatgaacagcctgagagatgaggacacggcactatattactgtgcaactgggatataccccaatgcttttggttactggggccagggcaccctggtcactgtctcctcag

SEQ ID NO: 82 - protein, 118 aa, Canis familiaris
EVQLVESGGDLVKPGGSLRLSCVASGFTFSSNYMSWIRQAPGKGLQWVSQISGDGIYTNYADAMKGRFTISRDNAKNTLYLQMNSLRDEDTALYYCATGIYPNAFGYWGQGTLVTVSS

SEQ ID NO: 83 - DNA, 319 bp, Canis familiaris
tcctatgtgctgacacagttgtcatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagcgctaatgtgttcggcggaggcacccatctgaccgtcctcg

SEQ ID NO: 84 - protein, 106 aa, Canis familiaris
SYVLTQLSSKNVTLKQPAHITCGGDNIGSKSVHWYQQKLGQAPVLIIYYDSSRPTGIPERFSGANSGNTATLTISGALAEDEADYYCQVWDSSANVFGGGTHLTVL

SEQ ID NO: 85 - protein, 7 aa, Canis familiaris
GFTFSSN

SEQ ID NO: 86 - protein, 6 aa, Canis familiaris
SGDGIY

SEQ ID NO: 87 - protein, 13 aa, Canis familiaris
CATGIYPNAFGYW

SEQ ID NO: 88 - protein, 11 aa, Canis familiaris
GGDNIGSKSVH

SEQ ID NO: 89 - protein, 7 aa, Canis familiaris
YDSSRPT

SEQ ID NO: 90 - protein, 11 aa, Canis familiaris
CQVWDSSANVF

SEQ ID NO: 91 - DNA, 361 bp, Canis familiaris
gaggtgcagctggtggagtctgggggagacctggtgaagcctggggggtccctgagaatttcctgtgtggcttctggattcaccttcagtagctaccacatgaactgggtccgccaggctccagggaaggggcttcagtgggtcgcacacattagcagtggtggaactttcacaagttatgcagacgctgtgaagggccgattcaccatctccagagacaacgccaagaacacgctctatcttcagatgatcagcctgagagccgaggacacggccgtgtattactgtgcgagtgggttgtttctagtagttgggggggggaactactggggccggggaaccctggtcaccgtctcctcag

SEQ ID NO: 92 - protein, 120 aa, Canis familiaris
EVQLVESGGDLVKPGGSLRISCVASGFTFSSYHMNWVRQAPGKGLQWVAHISSGGTFTSYADAVKGRFTISRDNAKNTLYLQMISLRAEDTAVYYCASGLFLVVGGGNYWGRGTLVTVSS

SEQ ID NO: 93 - DNA, 319 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattctgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 94 - protein, 106 aa, Canis familiaris
SYVLTQLPSKNVTLKQPAHITCGGDNIGSKSVHWYQQKLGQAPVLIIYSDSSRPTGIPERFSGANSGNTATLTISGALAEDEADYYCQVWDSSASVFGGGTHLTVL

SEQ ID NO: 95 - protein, 7 aa, Canis familiaris
GFTFSSY

SEQ ID NO: 96 - protein, 6 aa, Canis familiaris
SSGGTF

SEQ ID NO: 97 - protein, 15 aa, Canis familiaris
CASGLFLVVGGGNYW

SEQ ID NO: 98 - protein, 11 aa, Canis familiaris
GGDNIGSKSVH

SEQ ID NO: 99 - protein, 7 aa, Canis familiaris
SDSSRPT

SEQ ID NO: 100 - protein, 11 aa, Canis familiaris
CQVWDSSASVF

SEQ ID NO: 101 - DNA, 364 bp, Canis familiaris
gaggtgcaactggtggagtctgggggagacctggtgaagcctggggggtccctgagactttcctgtgtggcctctggattcaccttcagtaactaccacatgagctgggtccgccaggctccagggaaggggcttcagtgggtcgcatacattaacagtgatgggagagtcaccacctattcagacgctgtgaagggccgattcaccatctccagagacaacgccaagaacacgctgtgtcttcagatgaacagcctgagagccgaggacacggccgtgtattactgtgcgaggtggaggggtgggacttttggctatggtatggactactggggccatggcacctcactcttcgtgtcttcag

SEQ ID NO: 102 - protein, 121 aa, Canis familiaris
EVQLVESGGDLVKPGGSLRLSCVASGFTFSNYHMSWVRQAPGKGLQWVAYINSDGRVTTYSDAVKGRFTISRDNAKNTLCLQMNSLRAEDTAVYYCARWRGGTFGYGMDYWGHGTSLFVSS

SEQ ID NO: 103 - DNA, 325 bp, Canis familiaris
tcctatgtgctgacacagctgccatccaaaaatgtgaccctgaagcagccggcccacatcacctgtgggggagacaatattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 104 - protein, 108 aa, Canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKASVFGGGTHLTVL

SEQ ID NO: 105 - protein, 7 aa, Canis familiaris
GFTFSNY

SEQ ID NO: 106 - protein, 6 aa, Canis familiaris
NSDGRV

SEQ ID NO: 107 - protein, 16 aa, Canis familiaris
CARWRGGTFGYGMDYW

SEQ ID NO: 108 - protein, 11 aa, Canis familiaris
GGDNIGSKSVH

SEQ ID NO: 109 - protein, 7 aa, Canis familiaris
YDSSRPT

SEQ ID NO: 110 - protein, 13 aa, Canis familiaris
CQVWDSSAKASVF

SEQ ID NO: 111 - DNA, 364 bp, Canis familiaris
gaggtgcagctggtggagtctgggggagacctggtgaagcctggggggtccctgagactttcctgtgtggcctctggattcaccttcagtaactaccacatgagctgggtccgccaggctccagggaaggggcttcagtgggtcgcatacattaatagtgatggaagaattacaacctatgcagacgctgtgaagggccgattcaccatctccagagacaacgccaagaacacgctgtatcttcagatgaacagcctgagagtcgaggacacggccgtgtattactgtgcgaggtggaggggtgggacttttggctatggtatggactactggggccatggcacctcactcttcgtgtcctcag

SEQ ID NO: 112 - protein, 121 aa, Canis familiaris
EVQLVESGGDLVKPGGSLRLSCVASGFTFSNYHMSWVRQAPGKGLQWVAYINSDGRITTYADAVKGRFTISRDNAKNTLYLQMNSLRVEDTAVYYCARWRGGTFGYGMDYWGHGTSLFVSS

SEQ ID NO: 113 - DNA, 325 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 114 - protein, 108 aa, Canis familiaris; identical to SEQ ID NO: 104

SEQ ID NO: 115 - protein, 7 aa, Canis familiaris
GFTFSNY

SEQ ID NO: 116 - protein, 6 aa, Canis familiaris
NSDGRI

SEQ ID NO: 117 - protein, 16 aa, Canis familiaris
CARWRGGTFGYGMDYW

SEQ ID NO: 118 - protein, 11 aa, Canis familiaris
GGDNIGSKSVH

SEQ ID NO: 119 - protein, 7 aa, Canis familiaris
YDSSRPT

SEQ ID NO: 120 - protein, 13 aa, Canis familiaris
CQVWDSSAKASVF

SEQ ID NO: 121 - DNA, 361 bp, Canis familiaris
gaggtgcagctggtggagtctgggggagacctgatgaagcctggggggtccctgagactttcctgtgtggcctctggattcaccttccataactatcacatgaactgggtccgccaggctccagggaagggacttcagtgggtcgcacacattagcagtgatgggagattcataagctatgcagacactgtgaagggccgattcaccatctccagagacaacgccaagaacacgctgtatcttcagatgaacagcctgagagccgaggacacggccgtgtattactgtgcgaatggattgtttctggtacttgggggggagaactactggggccagggaaccctggtcaccgtctcctcag

SEQ ID NO: 122 - protein, 120 aa, Canis familiaris
EVQLVESGGDLMKPGGSLRLSCVASGFTFHNYHMNWVRQAPGKGLQWVAHISSDGRFISYADTVKGRFTISRDNAKNTLYLQMNSLRAEDTAVYYCANGLFLVLGGENYWGQGTLVTVSS

SEQ ID NO: 123 - DNA, 319 bp, Canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatattgattatctattatgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 124 - protein, 106 aa, Canis familiaris
SYVLTQLPSKNVTLKQPAHITCGGDNIGSKSVHWYQQKLGQAPILIIYYDSSRPTGIPERFSGANSGNTATLTISGALAEDEADYYCQVWDSSASVFGGGTHLTVL

SEQ ID NO: 125 - protein, 7 aa, Canis familiaris
GFTFHNY

SEQ ID NO: 126 - protein, 6 aa, Canis familiaris
SSDGRF

SEQ ID NO: 127 - protein, 15 aa, Canis familiaris
CANGLFLVLGGENYW

SEQ ID NO: 128 - protein, 11 aa, Canis familiaris
GGDNIGSKSVH

SEQ ID NO: 129 - protein, 7 aa, Canis familiaris
YDSSRPT

SEQ ID NO: 130 - protein, 11 aa, Canis familiaris
CQVWDSSASVF

SEQ ID NO: 131 - DNA, 355 bp, Canis familiaris
gaggtgctactgatggagtctgggggagacctggtgaagcctggggggtccctgagactctcctgtgtggcctctggattcaccttcagtagcaactacatgtactggatccgccaggctccagggaaggggctgcagtgggtctcacaaattagcggtgatggaagtttcacaaactacgcagacgctgtgaagggccgattcaccatctccagagacaatgccaagaacacactgtatctccagatgaacagcctgagagatgaggacacggcagttttttactgtgcaagtgggatataccccaatgcttttggttactggggccagggcaccctggtcactgtctcctcag

SEQ ID NO: 132 - protein, 118 aa, Canis familiaris
EVLLMESGGDLVKPGGSLRLSCVASGFTFSSNYMYWIRQAPGKGLQWVSQISGDGSFTNYADAVKGRFTISRDNAKNTLYLQMNSLRDEDTAVFYCASGIYPNAFGYWGQGTLVTVSS

SEQ ID NO: 133 - DNA, 319 bp, Canis familiaris
tcctatgtgctgacacagccgccatccaaaaatgtgaccctgaagcagccggcccacatcacctgtggcggagacaatattggaagtaaaagtgttcactggtatcagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgagggacagcagtgctaatgtgttcggcggaggcacccatctgaccgtcctcg

SEQ ID NO: 134 - protein, 106 aa, Canis familiaris
SYVLTQPPSKNVTLKQPAHITCGGDNIGSKSVHWYQQKLGQAPVLIIYYDSSRPTGIPERFSGANSGNTATLTISGALAEDEADYYCQVRDSSANVFGGGTHLTVL

SEQ ID NO: 135 - protein, 7 aa, Canis familiaris
GFTFSSN

SEQ ID NO: 136 - protein, 6 aa, Canis familiaris
SGDGSF

SEQ ID NO: 137 - protein, 13 aa, Canis familiaris
CASGIYPNAFGYW

SEQ ID NO: 138 - protein, 11 aa, Canis familiaris
GGDNIGSKSVH

SEQ ID NO: 139 - protein, 7 aa, Canis familiaris
YDSSRPT

SEQ ID NO: 140 - protein, 11 aa, Canis familiaris
CQVRDSSANVF

SEQ ID NO: 141 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 142 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLDPEDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 143 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 144 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 145 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 146 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 147 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 148 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 149 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPGMLGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 150 - protein, 335 aa, Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 151 - protein, 476 aa, Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 152 - DNA, 894 bp, Homo sapiens
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 153 - protein, 475 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 154 - protein, 471 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 155 - protein, 473 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 156 - protein, 471 aa, Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 157 - DNA, 737 bp, Canis familiaris
ctgctggcagagatgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggtggaaatagcttccggacccccatccaagaggagcatgctgacgccaactccaccctggccaagatctgagcagccagtgctcccac

SEQ ID NO: 158 - DNA, 842 bp, Canis familiaris
ctgctggcagagatgaggatgttcgtggagtccctgcggctcagcggtcctcactcagccctcctgctcctggggcttgtgctgggtgccgtagctgagcacaactgtttcgggaacacctaccccaaagacggcaagtgctgcaatgactgcccaccaggttatggaatggagagccgctgcagtaggagccatgacaccaaatgtcatcagtgtccatctggcttctacaatgaggctacaaattacgaaccctgcaagccctgcactcagtgcaatcagagaagtgggagtgaacccaagaggagatgcacacccacgcaggacaccatctgcagctgtaagccaggcacagagccccgggacggctacaagcgtggagtcgactgtgccccatgcccacccggacacttctccccaggggatgaccaggcctgcaagccctggaccaactgtaccttgatgggaaggcgtacaatgcagccggccagcaagagctcagacgctgtctgtgaggacaggagcctccccgccacactgccatgggagacccagagccccctgacccggccccctacccctcagcccactatggcctggcccaggacctcgcaggggcccttcacaccccctacggagccccccaggggcccccagctggctgctgtcctgggcttgggcctaggcttgctggcccccgtggcagccgcactggccttgctcctgcaccacagagcctggcggctgccccccggtggaaatagcttccggacccccatccaagaggagcatgctgacgccaactccaccctggccaagatctgagcagccagtgctcccac

SEQ ID NO: 159 - DNA, 620 bp, Canis familiaris
tgccacagttttcatctcccttacatctaaactgtgaagatggaaggggtccaacccctggaccagaatgtgggaaacacaccagggcgaagattccagaagaacaaggtattgctggtggcagccataattcaggggctgggtctgctcctgtgtttcacctacatctgcctgcacttctatgcttctcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctactgaggacctgagggctgcacctaagccaagca

SEQ ID NO: 160 - DNA, 666 bp, Artificial Sequence
  Secreted OX40L construct containing an IL-2 signal sequence, His-tag, AviTag, leucine zipper, a GS linker and the extracellular domain of OX40L, truncated at the extracellular- transmembrane domain junction.
atgtacaggatgcaactcctgtcttgcattgcactaagtcttgcacttgtcacaaacagtcaccaccaccaccaccacgggttgaatgatattttcgaagcacagaaaattgaatggcatgagcgtatgaaacagatcgaagataaaattgaagagatcttgagcaaaatctatcatatcgaaaacgaaattgcgcgtatcaaaaagctgattggcgaacgtggcggtggcagcggtggcggtagcggcggtggcagcgcttctcaggtgccgcctcagtatcctccaattcaaagtatcagagtacaatttaccaggtgtgagaatgagaaaggttgcatcatcacatccccaagcaaggatgaaactatgaaggtgcaagacaactcaatcatcattaactgtgatgggttttatctcatctccctgaagggttacttctctgaggagctcagcctcagcctttattaccgaaagggtcggggacccctcttctctctgagcaaggtcacatctgttgactccattggagtggcctatctggctttcaaggacaaagtctactttaatgtgaccactcacagtacctcctacaaagacatccaggtgaatggtggggaattgattctcattcatcaaaatcctggtggcttctgtgcctactga